SEQUENCE LISTING (28 sequences)

SEQ ID NO: 1 - DNA, 369 bp, Lama glama
  source 1..369: mol_type = other DNA
  misc_feature 1..369: note = DNA sequence encoding NbSYN87
caggtgcagctgcaggaaagcggcggcggcagcgtgcagaccggcggcagcctgcgcctgagctgcgtggcgagcggctatagcggctatatggcgtggtttcgccaggcgccgggcaaagaacgcgaaggcattgcggcgatttatcgcggcgataaaattacctattatgcgcatagcgtgcagggccgctttaccattagccaggcgaacgcgaaaaacaccgtgtatctgctgatgaacagcctgaaaccggaagataccgcgatttattattgcgcggcgcgccgcgtggtggcggatagcccgctgctgagcaaaacctatgcgtattggggccagggcacccaggtgaccgtgagcagctga

SEQ ID NO: 2 - DNA, 639 bp, Homo sapiens
  source 1..639: mol_type = other DNA
  misc_feature 1..639: note = DNA sequence encoding VHL
atgccccggagggcggagaactgggacgaggccgaggtaggcgcggaggaggcaggcgtcgaagagtacggccctgaagaagacggcggggaggagtcgggcgccgaggagtccggcccggaagagtccggcccggaggaactgggcgccgaggaggagatggaggccggtcggccgcggcccgtgctgcgctcggtgaactcgcgcgagccctcccaggtcatcttctgcaatcgcagtccgcgcgtcgtgctgcccgtatggctcaacttcgacggcgagccgcagccctacccaacgctgccgcctggcacgggccgccgcatccacagctaccgaggtcacctttggctcttcagagatgcagggacacacgatgggcttctggttaaccaaactgaattatttgtgccatctctcaatgttgacggacagcctatttttgccaatatcacactgccagtgtatactctgaaagagcgatgcctccaggttgtccggagcctagtcaagcctgagaattacaggagactggacatcgtcaggtcgctctacgaagatctggaagaccacccaaatgtgcagaaagacctggagcggctgacacaggagcgcattgcacatcaacggatgggagat

SEQ ID NO: 3 - DNA, 1032 bp, synthetic construct
  source 1..1032: mol_type = other DNA
  misc_feature 1..1032: note = DNA sequence encoding VHL-NbSYN87
atgccccggagggcggagaactgggacgaggccgaggtaggcgcggaggaggcaggcgtcgaagagtacggccctgaagaagacggcggggaggagtcgggcgccgaggagtccggcccggaagagtccggcccggaggaactgggcgccgaggaggagatggaggccggtcggccgcggcccgtgctgcgctcggtgaactcgcgcgagccctcccaggtcatcttctgcaatcgcagtccgcgcgtcgtgctgcccgtatggctcaacttcgacggcgagccgcagccctacccaacgctgccgcctggcacgggccgccgcatccacagctaccgaggtcacctttggctcttcagagatgcagggacacacgatgggcttctggttaaccaaactgaattatttgtgccatctctcaatgttgacggacagcctatttttgccaatatcacactgccagtgtatactctgaaagagcgatgcctccaggttgtccggagcctagtcaagcctgagaattacaggagactggacatcgtcaggtcgctctacgaagatctggaagaccacccaaatgtgcagaaagacctggagcggctgacacaggagcgcattgcacatcaacggatgggagatggtggaggcggaggtgaattcgccatgcaggtgcagctgcaggaaagcggcggcggcagcgtgcagaccggcggcagcctgcgcctgagctgcgtggcgagcggctatagcggctatatggcgtggtttcgccaggcgccgggcaaagaacgcgaaggcattgcggcgatttatcgcggcgataaaattacctattatgcgcatagcgtgcagggccgctttaccattagccaggcgaacgcgaaaaacaccgtgtatctgctgatgaacagcctgaaaccggaagataccgcgatttattattgcgcggcgcgccgcgtggtggcggatagcccgctgctgagcaaaacctatgcgtattggggccagggcacccaggtgaccgtgagcagc

SEQ ID NO: 4 - protein, 30 aa, synthetic construct
  REGION 1..30: note = Partial (C-terminal) sequence from alpha-synuclein
GILEDMPVDPDNEAYEMPSEEGYQDYEPEA

SEQ ID NO: 5 - protein, 12 aa, synthetic construct
  REGION 1..12: note = Putative epitope for NbSYN87
VDPDNEAYEMPS

SEQ ID NO: 6 - DNA, 1866 bp, Homo sapiens
  source 1..1866: mol_type = other DNA
  misc_feature 1..1866: note = DNA sequence encoding KLHL6
atgttgatggcaggacaaaggggcgcctggaccatgggtgatgtggtcgagaagagtttggaagggcccctggcaccttctacagatgagccctcccagaaaacaggagacttggtcgagatcttaaatggggaaaaggtcaaatttgacgacgcgggactctccttaattcttcagaatggcctggaaaccctgcgaatggaaaacgctctgacagatgtcatcttgtgtgtggacattcaggaattctcctgccaccgcgtggtgcttgccgcagccagcaactatttcagggccatgttctgcaacgatttaaaggaaaagtatgagaaaaggatcattattaaaggggttgatgctgagaccatgcacactctgttggactacacgtacaccagcaaggcgctgatcaccaagcagaatgtccagcgggtcctggaggctgctaacctcttccagttcctgcggatggtggatgcctgtgccagcttcctcactgaagccttgaacccagaaaactgcgttggaatactgaggctggctgacacacactcgctggacagtctaaagaagcaggttcagagttacatcattcaaaactttgtgcagattctgaactctgaggagtttcttgacctgcccgtggacactctgcaccacatcttgaagagtgatgacctttacgtgaccgaggaggctcaggtgtttgagaccgtgatgagctgggtccggcacaagccatcagaacgactctgcttactcccctatgtcctcgagaacgtgcgcttaccgcttctggacccgtggtactttgtggagacggtggaagcagatcctctcatcaggcagtgcccagaggtcttcccgctgctccaggaagccaggatgtaccacctttctggcaatgagatcatttcggaacgcaccaagcccaggatgcatgagttccagtctgaggtgttcatgatcattggcggctgcacgaaggatgaacggtttgtggcagaggtgacctgcctggaccccctgaggcgcagccgcctggaggtggccaagctcccgctaacagagcatgagctggagagtgagaataagaagtgggtggagtttgcatgcgtgacattgaaaaatgaggtctacatctcaggtggcaaagaaacacagcatgatgtttggaaatataattcttcgatcaacaagtggattcagattgagtatttaaacataggccgctggaggcataagatggtggtgttgggtggcaaggtctatgtgatcggaggctttgacggcttacagagaatcaacaacgtggagacctacgacccctttcacaactgctggtcagaggccgcacccctccttgtccatgtcagttcctttgcagccaccagccataagaagaagctgtatgtgatcgggggagggcccaatgggaaactggccacagacaagactcagtgttatgacccttccaccaacaagtggagtttgaaggcggccatgcccgtggaggctaaatgcatcaatgcagtgagtttccgggaccgcatctatgtcgttggtggggccatgagagcgctgtacgcctacagcccgctggaagacagctggtgcctggtgacccagctcagccacgagcgggccagctgcggtatcgcgccctgcaacaaccggctctacatcaccggcgggcgggacgagaagaacgaggttatcgccacggtgctgtgctgggaccccgaggcccagaaactgacagaggagtgcgtcctgccccggggcgtgtcgcaccacggcagcgtcaccatcaggaagtcgtacacccacatccgcaggatcgtgcccggagcagtgtctgtctga

SEQ ID NO: 7 - DNA, 1875 bp, Homo sapiens
  source 1..1875: mol_type = other DNA
  misc_feature 1..1875: note = DNA sequence encoding KEAP1
atgcagccagatcccaggcctagcggggctggggcctgctgccgattcctgcccctgcagtcacagtgccctgagggggcaggggacgcggtgatgtacgcctccactgagtgcaaggcggaggtgacgccctcccagcatggcaaccgcaccttcagctacaccctggaggatcataccaagcaggcctttggcatcatgaacgagctgcggctcagccagcagctgtgtgacgtcacactgcaggtcaagtaccaggatgcaccggccgcccagttcatggcccacaaggtggtgctggcctcatccagccctgtcttcaaggccatgttcaccaacgggctgcgggagcagggcatggaggtggtgtccattgagggtatccaccccaaggtcatggagcgcctcattgaattcgcctacacggcctccatctccatgggcgagaagtgtgtcctccacgtcatgaacggtgctgtcatgtaccagatcgacagcgttgtccgtgcctgcagtgacttcctggtgcagcagctggaccccagcaatgccatcggcatcgccaacttcgctgagcagattggctgtgtggagttgcaccagcgtgcccgggagtacatctacatgcattttggggaggtggccaagcaagaggagttcttcaacctgtcccactgccaactggtgaccctcatcagccgggacgacctgaacgtgcgctgcgagtccgaggtcttccacgcctgcatcaactgggtcaagtacgactgcgaacagcgacggttctacgtccaggcgctgctgcgggccgtgcgctgccactcgttgacgccgaacttcctgcagatgcagctgcagaagtgcgagatcctgcagtccgactcccgctgcaaggactacctggtcaagatcttcgaggagctcaccctgcacaagcccacgcaggtgatgccctgccgggcgcccaaggtgggccgcctgatctacaccgcgggcggctacttccgacagtcgctcagctacctggaggcttacaaccccagtgacggcacctggctccggttggcggacctgcaggtgccgcggagcggcctggccggctgcgtggtgggcgggctgttgtacgccgtgggcggcaggaacaactcgcccgacggcaacaccgactccagcgccctggactgttacaaccccatgaccaatcagtggtcgccctgcgcccccatgagcgtgccccgtaaccgcatcggggtgggggtcatcgatggccacatctatgccgtcggcggctcccacggctgcatccaccacaacagtgtggagaggtatgagccagagcgggatgagtggcacttggtggccccaatgctgacacgaaggatcggggtgggcgtggctgtcctcaatcgtctcctttatgccgtggggggctttgacgggacaaaccgccttaattcagctgagtgttactacccagagaggaacgagtggcgaatgatcacagcaatgaacaccatccgaagcggggcaggcgtctgcgtcctgcacaactgtatctatgctgctgggggctatgatggtcaggaccagctgaacagcgtggagcgctacgatgtggaaacagagacgtggactttcgtagcccccatgaagcaccggcgaagtgccctggggatcactgtccaccaggggagaatctacgtccttggaggctatgatggtcacacgttcctggacagtgtggagtgttacgacccagatacagacacctggagcgaggtgacccgaatgacatcgggccggagtggggtgggcgtggctgtcaccatggagccctgccggaagcagattgaccagcagaactgtacctgttga

SEQ ID NO: 8 - DNA, 1329 bp, Homo sapiens
  source 1..1329: mol_type = other DNA
  misc_feature 1..1329: note = DNA sequence encoding CRBN
atggccggcgaaggagatcagcaggacgctgcgcacaacatgggcaaccacctgccgctcctgcctgcagagagtgaggaagaagatgaaatggaagttgaagaccaggatagtaaagaagccaaaaaaccaaacatcataaattttgacaccagtctgccgacatcacatacatacctaggtgctgatatggaagaatttcatggcaggactttgcacgatgacgacagctgtcaggtgattccagttcttccacaagtgatgatgatcctgattcccggacagacattacctcttcagctttttcaccctcaagaagtcagtatggtgcggaatttaattcagaaagatagaacctttgctgttcttgcatacagcaatgtacaggaaagggaagcacagtttggaacaacagcagagatatatgcctatcgagaagaacaggattttggaattgagatagtgaaagtgaaagcaattggaagacaaaggttcaaagtccttgagctaagaacacagtcagatggaatccagcaagctaaagtgcaaattcttcccgaatgtgtgttgccttcaaccatgtctgcagttcaattagaatccctcaataagtgccagatatttccttcaaaacctgtctcaagagaagaccaatgttcatataaatggtggcagaaataccagaagagaaagtttcattgtgcaaatctaacttcatggcctcgctggctgtattccttatatgatgctgagaccttaatggacagaatcaagaaacagctacgtgaatgggatgaaaatctaaaagatgattctcttccttcaaatccaatagatttttcttacagagtagctgcttgtcttcctattgatgatgtattgagaattcagctccttaaaattggcagtgctatccagcgacttcgctgtgaattagacattatgaataaatgtacttccctttgctgtaaacaatgtcaagaaacagaaataacaaccaaaaatgaaatattcagtttatccttatgtgggccgatggcagcttatgtgaatcctcatggatatgtgcatgagacacttactgtgtataaggcttgcaacttgaatctgataggccggccttctacagaacacagctggtttcctgggtatgcctggactgttgcccagtgtaagatctgtgcaagccatattggatggaagtttacggccaccaaaaaagacatgtcacctcaaaaattttggggcttaacgcgatctgctctgttgcccacgatcccagacactgaagatgaaataagtccagacaaagtaatactttgcttgtaa

SEQ ID NO: 9 - DNA, 1221 bp, Homo sapiens
  source 1..1221: mol_type = other DNA
  misc_feature 1..1221: note = DNA sequence encoding KLHDC2
atggctgatggcaacgaggatctgcgggctgacgacttgcctgggccagccttcgagagctatgagtccatggagcttgcctgccccgctgagcgcagcggccacgtagccgtcagcgacgggcgccacatgttcgtctggggcggctacaagagtaatcaagtcagaggattatatgacttttatctgcctagagaagaactatggatctacaacatggagactggaagatggaaaaaaatcaacactgaaggtgatgttcctccttctatgtcaggaagctgtgctgtgtgtgtagacagggtgctgtacttgtttggaggacaccattcaagaggcaataccaataagttctacatgctggattcaaggtctacagacagagtgttacagtgggaaagaattgattgccaaggaattcctccatcatcaaaggacaaacttggtgtctgggtatataaaaacaagttaatattttttggagggtatggatatttgcctgaagataaagtattgggaacttttgaattcgatgaaacatctttttggaattcaagtcatccaagaggatggaatgatcatgtacatattttagatactgaaacatttacctggagccagcctataactactggtaaagcaccttcacctcgtgctgcccatgcctgtgcaactgtcggaaatagaggcttcgtgtttggaggcagatatcgagatgctagaatgaatgatcttcactatcttaatctggatacatgggagtggaatgaattaattccacaaggcatatgcccagttggtcgatcttggcactcactaacaccagtttcttcagatcatctttttctctttggaggatttaccactgataaacagccactaagtgatgcctggacttactgcatcagtaaaaatgaatggatacaatttaatcatccatataccgaaaaaccaaggttatggcacacagcttgtgccagcgatgaaggagaagtaattgtttttggtggatgtgccaacaacttgcttgtccatcacagagctgcacacagtaatgaaatactaatattttcagttcaaccaaaatctcttgtacggctaagcttagaagcagtcatttgctttaaagaaatgttagccaactcatggaactgccttccaaaacacttacttcacagtgttaatcagaggtttggtagtaacaacacttctggatcttaa

SEQ ID NO: 10 - DNA, 1539 bp, Homo sapiens
  source 1..1539: mol_type = other DNA
  misc_feature 1..1539: note = DNA sequence encoding TRAF3d56
atggagtcgagtaaaaagatggactctcctggcgcgctgcagactaacccgccgctaaagctgcacactgaccgcagtgctgggacgccagtttttgtccctgaacaaggaggttacaaggaaaagtttgtgaagaccgtggaggacaagtacaagtgtgagaagtgccacctggtgctgtgcagcccgaagcagaccgagtgtgggcaccgcttctgcgagagctgcatggcggccctgctgagctcttcaagtccaaaatgtacagcgtgtcaagagagcatcgttaaagataaggtgtttaaggataattgctgcaagagagaaattctggctcttcagatctattgtcggaatgaaagcagaggttgtgcagagcagttaatgctgggacatctgctggtgcatttaaaaaatgattgccattttgaagaacttccatgtgtgcgtcctgactgcaaagaaaaggtcttgaggaaagacctgcgagaccacgtggagaaggcgtgtaaataccgggaagccacatgcagccactgcaagagtcaggttccgatgatcgcgctgcagaaacacgaagacaccgactgtccctgcgtggtggtgtcctgccctcacaagtgcagcgtccagactctcctgaggagcgaggtttccttgttgcagaatgaaagtgtagaaaaaaacaagagcatacaaagtttgcacaatcagatatgtagctttgaaattgaaattgagagacaaaaggaaatgcttcgaaataatgaatccaaaatccttcatttacagcgagtgatagacagccaagcagagaaactgaaggagcttgacaaggagatccggcccttccggcagaactgggaggaagcagacagcatgaagagcagcgtggagtccctccagaaccgcgtgaccgagctggagagcgtggacaagagcgcggggcaagtggctcggaacacaggcctgctggagtcccagctgagccggcatgaccagatgctgagtgtgcacgacatccgcctagccgacatggacctgcgcttccaggtcctggagaccgccagctacaatggagtgctcatctggaagattcgcgactacaagcggcggaagcaggaggccgtcatggggaagaccctgtccctttacagccagcctttctacactggttactttggctataagatgtgtgccagggtctacctgaacggggacgggatggggaaggggacgcacttgtcgctgttttttgtcatcatgcgtggagaatatgatgccctgcttccttggccgtttaagcagaaagtgacactcatgctgatggatcaggggtcctctcgacgtcatttgggagatgcattcaagcccgaccccaacagcagcagcttcaagaagcccactggagagatgaatatcgcctctggctgcccagtctttgtggcccaaactgttctagaaaatgggacatatattaaagatgatacaatttttattaaagtcatagtggatacttcggatctgcccgatccctga

SEQ ID NO: 11 - protein, 140 aa, Homo sapiens
  REGION 1..140: note = Alpha-synuclein
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 12 - protein, 122 aa, Lama glama
  REGION 1..122: note = NbSYN87 amino acid sequence
QVQLQESGGGSVQTGGSLRLSCVASGYSGYMAWFRQAPGKEREGIAAIYRGDKITYYAHSVQGRFTISQANAKNTVYLLMNSLKPEDTAIYYCAARRVVADSPLLSKTYAYWGQGTQVTVSS

SEQ ID NO: 13 - protein, 213 aa, Homo sapiens
  REGION 1..213: note = VHL amino acid sequence
MPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGD

SEQ ID NO: 14 - protein, 344 aa, synthetic construct
  REGION 1..344: note = VHL-NbSYN87 amino acid sequence
MPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGDGGGGGEFAMQVQLQESGGGSVQTGGSLRLSCVASGYSGYMAWFRQAPGKEREGIAAIYRGDKITYYAHSVQGRFTISQANAKNTVYLLMNSLKPEDTAIYYCAARRVVADSPLLSKTYAYWGQGTQVTVSS

SEQ ID NO: 15 - protein, 621 aa, Homo sapiens
  REGION 1..621: note = KLHL6 amino acid sequence
MLMAGQRGAWTMGDVVEKSLEGPLAPSTDEPSQKTGDLVEILNGEKVKFDDAGLSLILQNGLETLRMENALTDVILCVDIQEFSCHRVVLAAASNYFRAMFCNDLKEKYEKRIIIKGVDAETMHTLLDYTYTSKALITKQNVQRVLEAANLFQFLRMVDACASFLTEALNPENCVGILRLADTHSLDSLKKQVQSYIIQNFVQILNSEEFLDLPVDTLHHILKSDDLYVTEEAQVFETVMSWVRHKPSERLCLLPYVLENVRLPLLDPWYFVETVEADPLIRQCPEVFPLLQEARMYHLSGNEIISERTKPRMHEFQSEVFMIIGGCTKDERFVAEVTCLDPLRRSRLEVAKLPLTEHELESENKKWVEFACVTLKNEVYISGGKETQHDVWKYNSSINKWIQIEYLNIGRWRHKMVVLGGKVYVIGGFDGLQRINNVETYDPFHNCWSEAAPLLVHVSSFAATSHKKKLYVIGGGPNGKLATDKTQCYDPSTNKWSLKAAMPVEAKCINAVSFRDRIYVVGGAMRALYAYSPLEDSWCLVTQLSHERASCGIAPCNNRLYITGGRDEKNEVIATVLCWDPEAQKLTEECVLPRGVSHHGSVTIRKSYTHIRRIVPGAVSV

SEQ ID NO: 16 - protein, 624 aa, Homo sapiens
  REGION 1..624: note = KEAP1 amino acid sequence
MQPDPRPSGAGACCRFLPLQSQCPEGAGDAVMYASTECKAEVTPSQHGNRTFSYTLEDHTKQAFGIMNELRLSQQLCDVTLQVKYQDAPAAQFMAHKVVLASSSPVFKAMFTNGLREQGMEVVSIEGIHPKVMERLIEFAYTASISMGEKCVLHVMNGAVMYQIDSVVRACSDFLVQQLDPSNAIGIANFAEQIGCVELHQRAREYIYMHFGEVAKQEEFFNLSHCQLVTLISRDDLNVRCESEVFHACINWVKYDCEQRRFYVQALLRAVRCHSLTPNFLQMQLQKCEILQSDSRCKDYLVKIFEELTLHKPTQVMPCRAPKVGRLIYTAGGYFRQSLSYLEAYNPSDGTWLRLADLQVPRSGLAGCVVGGLLYAVGGRNNSPDGNTDSSALDCYNPMTNQWSPCAPMSVPRNRIGVGVIDGHIYAVGGSHGCIHHNSVERYEPERDEWHLVAPMLTRRIGVGVAVLNRLLYAVGGFDGTNRLNSAECYYPERNEWRMITAMNTIRSGAGVCVLHNCIYAAGGYDGQDQLNSVERYDVETETWTFVAPMKHRRSALGITVHQGRIYVLGGYDGHTFLDSVECYDPDTDTWSEVTRMTSGRSGVGVAVTMEPCRKQIDQQNCTC

SEQ ID NO: 17 - protein, 442 aa, Homo sapiens [sequence omitted]

SEQ ID NO: 18 - protein, 406 aa, Homo sapiens
  REGION 1..406: note = KLHDC2 amino acid sequence
MADGNEDLRADDLPGPAFESYESMELACPAERSGHVAVSDGRHMFVWGGYKSNQVRGLYDFYLPREELWIYNMETGRWKKINTEGDVPPSMSGSCAVCVDRVLYLFGGHHSRGNTNKFYMLDSRSTDRVLQWERIDCQGIPPSSKDKLGVWVYKNKLIFFGGYGYLPEDKVLGTFEFDETSFWNSSHPRGWNDHVHILDTETFTWSQPITTGKAPSPRAAHACATVGNRGFVFGGRYRDARMNDLHYLNLDTWEWNELIPQGICPVGRSWHSLTPVSSDHLFLFGGFTTDKQPLSDAWTYCISKNEWIQFNHPYTEKPRLWHTACASDEGEVIVFGGCANNLLVHHRAAHSNEILIFSVQPKSLVRLSLEAVICFKEMLANSWNCLPKHLLHSVNQRFGSNNTSGS

SEQ ID NO: 19 - protein, 512 aa, Homo sapiens
  REGION 1..512: note = TRAF3d56 amino acid sequence
MESSKKMDSPGALQTNPPLKLHTDRSAGTPVFVPEQGGYKEKFVKTVEDKYKCEKCHLVLCSPKQTECGHRFCESCMAALLSSSSPKCTACQESIVKDKVFKDNCCKREILALQIYCRNESRGCAEQLMLGHLLVHLKNDCHFEELPCVRPDCKEKVLRKDLRDHVEKACKYREATCSHCKSQVPMIALQKHEDTDCPCVVVSCPHKCSVQTLLRSEVSLLQNESVEKNKSIQSLHNQICSFEIEIERQKEMLRNNESKILHLQRVIDSQAEKLKELDKEIRPFRQNWEEADSMKSSVESLQNRVTELESVDKSAGQVARNTGLLESQLSRHDQMLSVHDIRLADMDLRFQVLETASYNGVLIWKIRDYKRRKQEAVMGKTLSLYSQPFYTGYFGYKMCARVYLNGDGMGKGTHLSLFFVIMRGEYDALLPWPFKQKVTLMLMDQGSSRRHLGDAFKPDPNSSSFKKPTGEMNIASGCPVFVAQTVLENGTYIKDDTIFIKVIVDTSDLPDP

SEQ ID NO: 20 - DNA, 2256 bp, synthetic construct
  source 1..2256: mol_type = other DNA
  misc_feature 1..2256: note = NbSYN87 (5-G linker) KEAP1 DNA sequence
caggtgcagctgcaggaaagcggcggcggcagcgtgcagaccggcggcagcctgcgcctgagctgcgtggcgagcggctatagcggctatatggcgtggtttcgccaggcgccgggcaaagaacgcgaaggcattgcggcgatttatcgcggcgataaaattacctattatgcgcatagcgtgcagggccgctttaccattagccaggcgaacgcgaaaaacaccgtgtatctgctgatgaacagcctgaaaccggaagataccgcgatttattattgcgcggcgcgccgcgtggtggcggatagcccgctgctgagcaaaacctatgcgtattggggccagggcacccaggtgaccgtgagcagcggtggaggcggaggtatgcagccagatcccaggcctagcggggctggggcctgctgccgattcctgcccctgcagtcacagtgccctgagggggcaggggacgcggtgatgtacgcctccactgagtgcaaggcggaggtgacgccctcccagcatggcaaccgcaccttcagctacaccctggaggatcataccaagcaggcctttggcatcatgaacgagctgcggctcagccagcagctgtgtgacgtcacactgcaggtcaagtaccaggatgcaccggccgcccagttcatggcccacaaggtggtgctggcctcatccagccctgtcttcaaggccatgttcaccaacgggctgcgggagcagggcatggaggtggtgtccattgagggtatccaccccaaggtcatggagcgcctcattgaattcgcctacacggcctccatctccatgggcgagaagtgtgtcctccacgtcatgaacggtgctgtcatgtaccagatcgacagcgttgtccgtgcctgcagtgacttcctggtgcagcagctggaccccagcaatgccatcggcatcgccaacttcgctgagcagattggctgtgtggagttgcaccagcgtgcccgggagtacatctacatgcattttggggaggtggccaagcaagaggagttcttcaacctgtcccactgccaactggtgaccctcatcagccgggacgacctgaacgtgcgctgcgagtccgaggtcttccacgcctgcatcaactgggtcaagtacgactgcgaacagcgacggttctacgtccaggcgctgctgcgggccgtgcgctgccactcgttgacgccgaacttcctgcagatgcagctgcagaagtgcgagatcctgcagtccgactcccgctgcaaggactacctggtcaagatcttcgaggagctcaccctgcacaagcccacgcaggtgatgccctgccgggcgcccaaggtgggccgcctgatctacaccgcgggcggctacttccgacagtcgctcagctacctggaggcttacaaccccagtgacggcacctggctccggttggcggacctgcaggtgccgcggagcggcctggccggctgcgtggtgggcgggctgttgtacgccgtgggcggcaggaacaactcgcccgacggcaacaccgactccagcgccctggactgttacaaccccatgaccaatcagtggtcgccctgcgcccccatgagcgtgccccgtaaccgcatcggggtgggggtcatcgatggccacatctatgccgtcggcggctcccacggctgcatccaccacaacagtgtggagaggtatgagccagagcgggatgagtggcacttggtggccccaatgctgacacgaaggatcggggtgggcgtggctgtcctcaatcgtctcctttatgccgtggggggctttgacgggacaaaccgccttaattcagctgagtgttactacccagagaggaacgagtggcgaatgatcacagcaatgaacaccatccgaagcggggcaggcgtctgcgtcctgcacaactgtatctatgctgctgggggctatgatggtcaggaccagctgaacagcgtggagcgctacgatgtggaaacagagacgtggactttcgtagcccccatgaagcaccggcgaagtgccctggggatcactgtccaccaggggagaatctacgtccttggaggctatgatggtcacacgttcctggacagtgtggagtgttacgacccagatacagacacctggagcgaggtgacccgaatgacatcgggccggagtggggtgggcgtggctgtcaccatggagccctgccggaagcagattgaccagcagaactgtacctgttga

SEQ ID NO: 21 - protein, 751 aa, synthetic construct
  REGION 1..751: note = NbSYN87 (5-G linker) KEAP1
QVQLQESGGGSVQTGGSLRLSCVASGYSGYMAWFRQAPGKEREGIAAIYRGDKITYYAHSVQGRFTISQANAKNTVYLLMNSLKPEDTAIYYCAARRVVADSPLLSKTYAYWGQGTQVTVSSGGGGGMQPDPRPSGAGACCRFLPLQSQCPEGAGDAVMYASTECKAEVTPSQHGNRTFSYTLEDHTKQAFGIMNELRLSQQLCDVTLQVKYQDAPAAQFMAHKVVLASSSPVFKAMFTNGLREQGMEVVSIEGIHPKVMERLIEFAYTASISMGEKCVLHVMNGAVMYQIDSVVRACSDFLVQQLDPSNAIGIANFAEQIGCVELHQRAREYIYMHFGEVAKQEEFFNLSHCQLVTLISRDDLNVRCESEVFHACINWVKYDCEQRRFYVQALLRAVRCHSLTPNFLQMQLQKCEILQSDSRCKDYLVKIFEELTLHKPTQVMPCRAPKVGRLIYTAGGYFRQSLSYLEAYNPSDGTWLRLADLQVPRSGLAGCVVGGLLYAVGGRNNSPDGNTDSSALDCYNPMTNQWSPCAPMSVPRNRIGVGVIDGHIYAVGGSHGCIHHNSVERYEPERDEWHLVAPMLTRRIGVGVAVLNRLLYAVGGFDGTNRLNSAECYYPERNEWRMITAMNTIRSGAGVCVLHNCIYAAGGYDGQDQLNSVERYDVETETWTFVAPMKHRRSALGITVHQGRIYVLGGYDGHTFLDSVECYDPDTDTWSEVTRMTSGRSGVGVAVTMEPCRKQIDQQNCTC

SEQ ID NO: 22 - DNA, 2253 bp, synthetic construct
  source 1..2253: mol_type = other DNA
  misc_feature 1..2253: note = KEAP1 (5-G linker) NbSYN87 DNA sequence
atgcagccagatcccaggcctagcggggctggggcctgctgccgattcctgcccctgcagtcacagtgccctgagggggcaggggacgcggtgatgtacgcctccactgagtgcaaggcggaggtgacgccctcccagcatggcaaccgcaccttcagctacaccctggaggatcataccaagcaggcctttggcatcatgaacgagctgcggctcagccagcagctgtgtgacgtcacactgcaggtcaagtaccaggatgcaccggccgcccagttcatggcccacaaggtggtgctggcctcatccagccctgtcttcaaggccatgttcaccaacgggctgcgggagcagggcatggaggtggtgtccattgagggtatccaccccaaggtcatggagcgcctcattgaattcgcctacacggcctccatctccatgggcgagaagtgtgtcctccacgtcatgaacggtgctgtcatgtaccagatcgacagcgttgtccgtgcctgcagtgacttcctggtgcagcagctggaccccagcaatgccatcggcatcgccaacttcgctgagcagattggctgtgtggagttgcaccagcgtgcccgggagtacatctacatgcattttggggaggtggccaagcaagaggagttcttcaacctgtcccactgccaactggtgaccctcatcagccgggacgacctgaacgtgcgctgcgagtccgaggtcttccacgcctgcatcaactgggtcaagtacgactgcgaacagcgacggttctacgtccaggcgctgctgcgggccgtgcgctgccactcgttgacgccgaacttcctgcagatgcagctgcagaagtgcgagatcctgcagtccgactcccgctgcaaggactacctggtcaagatcttcgaggagctcaccctgcacaagcccacgcaggtgatgccctgccgggcgcccaaggtgggccgcctgatctacaccgcgggcggctacttccgacagtcgctcagctacctggaggcttacaaccccagtgacggcacctggctccggttggcggacctgcaggtgccgcggagcggcctggccggctgcgtggtgggcgggctgttgtacgccgtgggcggcaggaacaactcgcccgacggcaacaccgactccagcgccctggactgttacaaccccatgaccaatcagtggtcgccctgcgcccccatgagcgtgccccgtaaccgcatcggggtgggggtcatcgatggccacatctatgccgtcggcggctcccacggctgcatccaccacaacagtgtggagaggtatgagccagagcgggatgagtggcacttggtggccccaatgctgacacgaaggatcggggtgggcgtggctgtcctcaatcgtctcctttatgccgtggggggctttgacgggacaaaccgccttaattcagctgagtgttactacccagagaggaacgagtggcgaatgatcacagcaatgaacaccatccgaagcggggcaggcgtctgcgtcctgcacaactgtatctatgctgctgggggctatgatggtcaggaccagctgaacagcgtggagcgctacgatgtggaaacagagacgtggactttcgtagcccccatgaagcaccggcgaagtgccctggggatcactgtccaccaggggagaatctacgtccttggaggctatgatggtcacacgttcctggacagtgtggagtgttacgacccagatacagacacctggagcgaggtgacccgaatgacatcgggccggagtggggtgggcgtggctgtcaccatggagccctgccggaagcagattgaccagcagaactgtacctgtggtggaggcggaggtcaggtgcagctgcaggaaagcggcggcggcagcgtgcagaccggcggcagcctgcgcctgagctgcgtggcgagcggctatagcggctatatggcgtggtttcgccaggcgccgggcaaagaacgcgaaggcattgcggcgatttatcgcggcgataaaattacctattatgcgcatagcgtgcagggccgctttaccattagccaggcgaacgcgaaaaacaccgtgtatctgctgatgaacagcctgaaaccggaagataccgcgatttattattgcgcggcgcgccgcgtggtggcggatagcccgctgctgagcaaaacctatgcgtattggggccagggcacccaggtgaccgtgagcagc

SEQ ID NO: 23 - protein, 751 aa, synthetic construct
  REGION 1..751: note = KEAP1 (5-G linker) NbSYN87 amino acid sequence
MQPDPRPSGAGACCRFLPLQSQCPEGAGDAVMYASTECKAEVTPSQHGNRTFSYTLEDHTKQAFGIMNELRLSQQLCDVTLQVKYQDAPAAQFMAHKVVLASSSPVFKAMFTNGLREQGMEVVSIEGIHPKVMERLIEFAYTASISMGEKCVLHVMNGAVMYQIDSVVRACSDFLVQQLDPSNAIGIANFAEQIGCVELHQRAREYIYMHFGEVAKQEEFFNLSHCQLVTLISRDDLNVRCESEVFHACINWVKYDCEQRRFYVQALLRAVRCHSLTPNFLQMQLQKCEILQSDSRCKDYLVKIFEELTLHKPTQVMPCRAPKVGRLIYTAGGYFRQSLSYLEAYNPSDGTWLRLADLQVPRSGLAGCVVGGLLYAVGGRNNSPDGNTDSSALDCYNPMTNQWSPCAPMSVPRNRIGVGVIDGHIYAVGGSHGCIHHNSVERYEPERDEWHLVAPMLTRRIGVGVAVLNRLLYAVGGFDGTNRLNSAECYYPERNEWRMITAMNTIRSGAGVCVLHNCIYAAGGYDGQDQLNSVERYDVETETWTFVAPMKHRRSALGITVHQGRIYVLGGYDGHTFLDSVECYDPDTDTWSEVTRMTSGRSGVGVAVTMEPCRKQIDQQNCTCGGGGGQVQLQESGGGSVQTGGSLRLSCVASGYSGYMAWFRQAPGKEREGIAAIYRGDKITYYAHSVQGRFTISQANAKNTVYLLMNSLKPEDTAIYYCAARRVVADSPLLSKTYAYWGQGTQVTVSS

SEQ ID NO: 24 - DNA, 1599 bp, synthetic construct
  source 1..1599: mol_type = other DNA
  misc_feature 1..1599: note = KLHDC2 (5-G linker) NbSYN87 DNA sequence
atggctgatggcaacgaggatctgcgggctgacgacttgcctgggccagccttcgagagctatgagtccatggagcttgcctgccccgctgagcgcagcggccacgtagccgtcagcgacgggcgccacatgttcgtctggggcggctacaagagtaatcaagtcagaggattatatgacttttatctgcctagagaagaactatggatctacaacatggagactggaagatggaaaaaaatcaacactgaaggtgatgttcctccttctatgtcaggaagctgtgctgtgtgtgtagacagggtgctgtacttgtttggaggacaccattcaagaggcaataccaataagttctacatgctggattcaaggtctacagacagagtgttacagtgggaaagaattgattgccaaggaattcctccatcatcaaaggacaaacttggtgtctgggtatataaaaacaagttaatattttttggagggtatggatatttgcctgaagataaagtattgggaacttttgaattcgatgaaacatctttttggaattcaagtcatccaagaggatggaatgatcatgtacatattttagatactgaaacatttacctggagccagcctataactactggtaaagcaccttcacctcgtgctgcccatgcctgtgcaactgtcggaaatagaggcttcgtgtttggaggcagatatcgagatgctagaatgaatgatcttcactatcttaatctggatacatgggagtggaatgaattaattccacaaggcatatgcccagttggtcgatcttggcactcactaacaccagtttcttcagatcatctttttctctttggaggatttaccactgataaacagccactaagtgatgcctggacttactgcatcagtaaaaatgaatggatacaatttaatcatccatataccgaaaaaccaaggttatggcacacagcttgtgccagcgatgaaggagaagtaattgtttttggtggatgtgccaacaacttgcttgtccatcacagagctgcacacagtaatgaaatactaatattttcagttcaaccaaaatctcttgtacggctaagcttagaagcagtcatttgctttaaagaaatgttagccaactcatggaactgccttccaaaacacttacttcacagtgttaatcagaggtttggtagtaacaacacttctggatctggtggaggcggaggtcaggtgcagctgcaggaaagcggcggcggcagcgtgcagaccggcggcagcctgcgcctgagctgcgtggcgagcggctatagcggctatatggcgtggtttcgccaggcgccgggcaaagaacgcgaaggcattgcggcgatttatcgcggcgataaaattacctattatgcgcatagcgtgcagggccgctttaccattagccaggcgaacgcgaaaaacaccgtgtatctgctgatgaacagcctgaaaccggaagataccgcgatttattattgcgcggcgcgccgcgtggtggcggatagcccgctgctgagcaaaacctatgcgtattggggccagggcacccaggtgaccgtgagcagc

SEQ ID NO: 25 - protein, 533 aa, synthetic construct
  REGION 1..533: note = KLHDC2 (5-G linker) NbSYN87 amino acid sequence
MADGNEDLRADDLPGPAFESYESMELACPAERSGHVAVSDGRHMFVWGGYKSNQVRGLYDFYLPREELWIYNMETGRWKKINTEGDVPPSMSGSCAVCVDRVLYLFGGHHSRGNTNKFYMLDSRSTDRVLQWERIDCQGIPPSSKDKLGVWVYKNKLIFFGGYGYLPEDKVLGTFEFDETSFWNSSHPRGWNDHVHILDTETFTWSQPITTGKAPSPRAAHACATVGNRGFVFGGRYRDARMNDLHYLNLDTWEWNELIPQGICPVGRSWHSLTPVSSDHLFLFGGFTTDKQPLSDAWTYCISKNEWIQFNHPYTEKPRLWHTACASDEGEVIVFGGCANNLLVHHRAAHSNEILIFSVQPKSLVRLSLEAVICFKEMLANSWNCLPKHLLHSVNQRFGSNNTSGSGGGGGQVQLQESGGGSVQTGGSLRLSCVASGYSGYMAWFRQAPGKEREGIAAIYRGDKITYYAHSVQGRFTISQANAKNTVYLLMNSLKPEDTAIYYCAARRVVADSPLLSKTYAYWGQGTQVTVSS

SEQ ID NO: 26 - DNA, 2244 bp, synthetic construct
  source 1..2244: mol_type = other DNA
  misc_feature 1..2244: note = KLHL6 (5-G linker) NbSYN87 DNA sequence
atgttgatggcaggacaaaggggcgcctggaccatgggtgatgtggtcgagaagagtttggaagggcccctggcaccttctacagatgagccctcccagaaaacaggagacttggtcgagatcttaaatggggaaaaggtcaaatttgacgacgcgggactctccttaattcttcagaatggcctggaaaccctgcgaatggaaaacgctctgacagatgtcatcttgtgtgtggacattcaggaattctcctgccaccgcgtggtgcttgccgcagccagcaactatttcagggccatgttctgcaacgatttaaaggaaaagtatgagaaaaggatcattattaaaggggttgatgctgagaccatgcacactctgttggactacacgtacaccagcaaggcgctgatcaccaagcagaatgtccagcgggtcctggaggctgctaacctcttccagttcctgcggatggtggatgcctgtgccagcttcctcactgaagccttgaacccagaaaactgcgttggaatactgaggctggctgacacacactcgctggacagtctaaagaagcaggttcagagttacatcattcaaaactttgtgcagattctgaactctgaggagtttcttgacctgcccgtggacactctgcaccacatcttgaagagtgatgacctttacgtgaccgaggaggctcaggtgtttgagaccgtgatgagctgggtccggcacaagccatcagaacgactctgcttactcccctatgtcctcgagaacgtgcgcttaccgcttctggacccgtggtactttgtggagacggtggaagcagatcctctcatcaggcagtgcccagaggtcttcccgctgctccaggaagccaggatgtaccacctttctggcaatgagatcatttcggaacgcaccaagcccaggatgcatgagttccagtctgaggtgttcatgatcattggcggctgcacgaaggatgaacggtttgtggcagaggtgacctgcctggaccccctgaggcgcagccgcctggaggtggccaagctcccgctaacagagcatgagctggagagtgagaataagaagtgggtggagtttgcatgcgtgacattgaaaaatgaggtctacatctcaggtggcaaagaaacacagcatgatgtttggaaatataattcttcgatcaacaagtggattcagattgagtatttaaacataggccgctggaggcataagatggtggtgttgggtggcaaggtctatgtgatcggaggctttgacggcttacagagaatcaacaatgtggagacctacgacccctttcacaactgctggtcagaggccgcacccctccttgtccatgtcagttcctttgcagccaccagccataagaagaagctgtatgtgatcgggggagggcccaatgggaaactggccacagacaagactcagtgttatgacccttccaccaacaagtggagtttgaaggcggccatgcccgtggaggctaaatgcatcaatgcagtgagtttccgggaccgcatctatgtcgttggtggggccatgagagcgctgtacgcctacagcccgctggaagacagctggtgcctggtgacccagctcagccacgagcgggccagctgcggtatcgcgccctgcaacaaccggctctacatcaccggcgggcgggacgagaagaacgaggttatcgccacggtgctgtgctgggaccccgaggcccagaaactgacagaggagtgcgtcctgccccggggcgtgtcgcaccacggcagcgtcaccatcaggaagtcgtacacccacatccgcaggatcgtgcccggagcagtgtctgtcggtggaggcggaggtcaggtgcagctgcaggaaagcggcggcggcagcgtgcagaccggcggcagcctgcgcctgagctgcgtggcgagcggctatagcggctatatggcgtggtttcgccaggcgccgggcaaagaacgcgaaggcattgcggcgatttatcgcggcgataaaattacctattatgcgcatagcgtgcagggccgctttaccattagccaggcgaacgcgaaaaacaccgtgtatctgctgatgaacagcctgaaaccggaagataccgcgatttattattgcgcggcgcgccgcgtggtggcggatagcccgctgctgagcaaaacctatgcgtattggggccagggcacccaggtgaccgtgagcagc

SEQ ID NO: 27 - protein, 748 aa, synthetic construct
  REGION 1..748: note = KLHL6 (5-G linker) NbSYN87 amino acid sequence
MLMAGQRGAWTMGDVVEKSLEGPLAPSTDEPSQKTGDLVEILNGEKVKFDDAGLSLILQNGLETLRMENALTDVILCVDIQEFSCHRVVLAAASNYFRAMFCNDLKEKYEKRIIIKGVDAETMHTLLDYTYTSKALITKQNVQRVLEAANLFQFLRMVDACASFLTEALNPENCVGILRLADTHSLDSLKKQVQSYIIQNFVQILNSEEFLDLPVDTLHHILKSDDLYVTEEAQVFETVMSWVRHKPSERLCLLPYVLENVRLPLLDPWYFVETVEADPLIRQCPEVFPLLQEARMYHLSGNEIISERTKPRMHEFQSEVFMIIGGCTKDERFVAEVTCLDPLRRSRLEVAKLPLTEHELESENKKWVEFACVTLKNEVYISGGKETQHDVWKYNSSINKWIQIEYLNIGRWRHKMVVLGGKVYVIGGFDGLQRINNVETYDPFHNCWSEAAPLLVHVSSFAATSHKKKLYVIGGGPNGKLATDKTQCYDPSTNKWSLKAAMPVEAKCINAVSFRDRIYVVGGAMRALYAYSPLEDSWCLVTQLSHERASCGIAPCNNRLYITGGRDEKNEVIATVLCWDPEAQKLTEECVLPRGVSHHGSVTIRKSYTHIRRIVPGAVSVGGGGGQVQLQESGGGSVQTGGSLRLSCVASGYSGYMAWFRQAPGKEREGIAAIYRGDKITYYAHSVQGRFTISQANAKNTVYLLMNSLKPEDTAIYYCAARRVVADSPLLSKTYAYWGQGTQVTVSS

SEQ ID NO: 28 - protein, 5 aa, synthetic construct
  REGION 1..5: note = Linker protein
GGGGG

(19) United States
(12) Patent Application Publication
(10) Pub. No.: US 2025/0059527 A1
(43) Pub. Date: Feb. 20, 2025

(54) TARGETED DEGRADATION OF ALPHA-SYNUCLEIN

(71) Applicant: UNIVERSITY OF DUNDEE, Dundee (GB)

(72) Inventor: Gopal SAPKOTA, Dundee (GB)

(73) Assignee: UNIVERSITY OF DUNDEE, Dundee (GB)

(21) Appl. No.: 18/720,366

(22) PCT Filed: Dec. 15, 2022
(86) PCT No.: PCT/GB2022/053257

§ 371 Date: Jun. 14, 2024

(30) Foreign Application Priority Data
Dec. 16, 2021 (GB) 2118272.0

(51) Int. Cl. C12N 9/00; A61K 38/00; A61K 47/68; C07K 16/18; C12N 15/86

(52) U.S. Cl. CPC C12N 9/93; A61K 47/6843; C07K 16/18; C12N 15/86; A61K 38/00; C07K 2317/569; C07K 2319/95; C12N 2740/10043; C12Y 603/02019

(57) ABSTRACT

The present invention relates to a proteasomal degradation protein complex that comprises an E3 ubiquitin ligase component tethered to an α-synuclein-specific polypeptide binder for degradation of α-synuclein. The present invention also comprises a mechanism of action, composition and related methods for the treatment of neurological disorders including synucleopathies.

### FIELD OF THE INVENTION

The present invention relates to a proteasomal degradation protein complex comprising an E3 ligase substrate receptor linked to a specific binder of a target protein. In particular, the present invention relates to a proteasomal degradation protein complex comprising an E3 ligase linked a specific polypeptide binder for the degradation of endogenous α-synuclein and associated methods and uses in the treatment of neurodegenerative disorders and suchlike.

### BACKGROUND OF THE INVENTION

The following discussion is provided to aid the reader in understanding the disclosure and does not constitute any admission as to the contents or relevance of the prior art.

Parkinson's disease is the second most common neurodegenerative disorder, affecting 1:500 people in the UK. Proteinaceous intracellular inclusions known as Lewy Bodies are a hallmark characteristic of Parkinson's disease, which are found primarily within dopaminergic neurones. The main component of Lewy Bodies is a small 140 kDa protein called α-synuclein, encoded by the SNCA gene.

The presence of α-synuclein in intracellular inclusions has also been found in a variety of other neurodegenerative disorders including Dementia with Lewy Bodies and multiple system atrophy, collectively termed the synucleinopathies. Synucleinopathies are associated with various SNCA mutations, including A53T, A30P, H50Q, E46K, G51D and A53E mutants, or duplications and triplications of the SNCA gene. Furthermore, aggregation of α-synuclein in dopaminergic neurones is associated with the pathogenesis and progression of synucleinopathies and therefore a potential target for therapeutic interventions. However, α-synuclein has been deemed as undruggable by conventional small molecule methods. Therefore, there is a need for developing new methods and therapeutics to target α-synuclein in synucleoinopathies.

The affinity-directed protein missile (AdPROM) system technology was developed to target endogenous proteins for proteolysis (Fulcher L J et al., 2017, Targeting endogenous proteins for degradation through the affinity-directed protein missile system. Open Biol. 7: 170066). The AdPROM system exploits the ubiquitin-proteasome system by linking an E3 ligase component such as the CUL2-CRL substrate receptor von-Hippel Lindau tumour suppressor (VHL) to a target-specific polypeptide binder such an antibody or fragment thereof. By tethering an E3 ligase component to a target-specific peptide binder, the system can be exploited to selectively recruit target proteins to the CUL2-CRL machinery, which then facilitates the ubiquitylation and subsequent degradation of the protein via the proteasome with high selectivity and specificity.

There are currently no effective therapies for synucleinopathies and no known therapeutics that effectively target α-synuclein. Therefore, there is a need for providing a novel method of degrading α-synuclein and providing a therapeutic system for the treatment of synucleinopathies.

Accordingly, in at least some aspects the present disclosure describes a proteasomal degradation protein complex aimed to target endogenous α-synuclein for proteasomal degradation and provide a highly selective and specific target for the therapeutic treatment for synucleinopathies.

### SUMMARY OF THE INVENTION

In a first aspect of the present invention, there is provided a proteasomal degradation protein complex comprising an E3 ubiquitin ligase component tethered to an α-synuclein-specific polypeptide binder, wherein the proteasomal degradation protein complex is capable of targeting α-synuclein for proteasomal degradation.

Degrading α-synuclein is achieved by targeting the α-synuclein for degradation by the proteasomal system in a cell. Preferably, in some embodiments α-synuclein is specifically targeted for degradation by the ubiquitin-mediated proteasomal degradation system.

### α-Synuclein-Specific Polypeptide Binders:

Suitably, in some embodiments the α-synuclein-specific polypeptide binder is an antibody, an antibody fragment, a monobody and/or a nanobody. However, it will be apparent to the skilled person that other types of α-synuclein-specific polypeptide binders can be used, e.g. those based on various scaffold proteins. For use in the present invention the α-synuclein-specific polypeptide binder should be able to bind to α-synuclein in a cellular context, i.e. intracellularly, thereby presenting α-synuclein for ubiquitination by the E3 ubiquitin ligase component of the complex.

The amino acid sequence of α-synuclein (SEQ ID NO: 11) can be divided into 3 regions. The N-terminal domain (residues 1-60), the NAC domain (residues 61-95) and the C-terminal domain (residues 96-140) (Farzadfard, A, et al., 2022).

In some embodiments, the α-synuclein-specific polypeptide binder is an antibody specific to a target epitope in the C-terminal region (residues 96-140 of SEQ ID NO: 11) of the α-synuclein protein. Suitably, in some embodiments the α-synuclein-specific polypeptide binder is a nanobody specific to a target epitope in the C-terminal region of the α-synuclein protein.

In some embodiments, the α-synuclein-specific polypeptide binder is an antibody specific to a target epitope in the N-terminal region (residues 1-60 of SEQ ID NO: 11) of the α-synuclein protein. Suitably, in some embodiments the α-synuclein-specific polypeptide binder is a nanobody specific to a target epitope in the N-terminal region of the α-synuclein protein.

In some embodiments, the α-synuclein-specific polypeptide binder is an antibody specific to a target epitope in the NAC domain (residues 61-95 of SEQ ID NO: 11) of the α-synuclein protein. Suitably, in some embodiments the α-synuclein-specific polypeptide binder is a nanobody specific to a target epitope in the NAC domain of the α-synuclein protein.

In some embodiments the α-synuclein-specific polypeptide binder is the nanobody NbSYN87 or a functional variant thereof. In some embodiments, the α-synuclein-specific polypeptide binder is the nanobody NbSYN87 according to SEQ ID NO: 12 or a functional variant thereof.

NbSYN87 has been shown to be a particularly effective α-synuclein-specific polypeptide binder for use in the present invention. A functional variant of NbSYN87 suitably comprises a sequence that is at least 60% identical to wild type NbSYN87 (SEQ ID NO:12), more preferably at least 70%, 80%, 90%, 95% or 99% identical to wild type NbSYN87. A functional variant of NbSYN87 suitably comprises a sequence that is at least 60% identical to SEQ ID NO: 12, more preferably at least 70%, 80%, 90%, 95% or 99% identical to SEQ ID NO: 12. In some embodiments, a functional variant of NbSYN87 may be encoded by a DNA sequence that is at least 60% identical to SEQ ID NO:1, more preferably at least 70%, 80%, 90%, 95% or 99% identical to SEQ ID NO: 1. Preferably the functional variant of NbSYN87 retains a similar or higher affinity for α-synuclein compared to wild type NbSYN87, e.g. at least 50%, 60%, 70%, 80%, 90% or 100% of the affinity for α-synuclein as wild type NbSYN87.

While NbSYN87 has been shown to function particularly well in the present invention, the skilled person will appreciate that other α-synuclein-specific polypeptide binders can be used. In some embodiments, the α-synuclein-specific polypeptide binder binds α-synuclein with a similar or higher affinity as NbSYN87 (e.g. at least 50%, 60%, 70%, 80%, 90% or 100% of the affinity for α-synuclein as NbSYN87).

The ability of any putative α-synuclein-specific polypeptide binder to function in the context of the present invention can be assessed using the methodologies described herein. For example, as an initial screen to test candidate α-synuclein-specific polypeptide binders, the interaction between any α-synuclein-specific polypeptide binder and α-synuclein can be carried out by immunoprecipitation (IP) (e.g. as described in Example 2 below). Thereafter, assuming the α-synuclein-specific polypeptide binder performs as desired in IP, its ability to specifically bind α-synuclein in a cellular context can be assessed (e.g. using immunofluorescence as described in Example 2 below). The activity of α-synuclein-specific polypeptide binder in the context of a proteasomal degradation protein complex of the present invention can readily be assessed by substituting it for NbSYN87 in the various specific examples discussed below.

Without wishing to be bound by theory, it is believed that α-synuclein-specific polypeptide binders targeting the same epitope on α-synuclein as NbSYN87 may be particularly beneficial in providing effective targeting of α-synuclein for degradation. Accordingly, in some embodiments, the specific polypeptide binder binds with a similar or higher affinity as NbSYN87 for the same target epitope (e.g. at least 50%, 60%, 70%, 80%, 90% or 100% of the affinity of NbSYN87 for the same target epitope as NbSYN87). High selectivity of the polypeptide binder for α-synuclein appears to be beneficial for the function of the proteasomal degradation protein complex. In some embodiments the α-synuclein-specific polypeptide competes with NbSYN87 for binding to the same target epitope (e.g. in a competitive binding assay), e.g. having a similar or higher affinity for the target epitope as NbSYN87.

The putative epitope for NbSYN87 is underlined in the following partial (C-terminal) sequence from α-synuclein: 111-GILEDMPVDPDNEAYEMPSEEGYQDYEPEA-140 (SEQ ID NO: 4), i.e. amino acids 118-129 of α-synuclein. Further details of NbSYN87 can be found in Guilliams, et al, Journal of Molecular Biology, Volume 425, Issue 14, 24 Jul. 2013, Pages 2397-2411.

A nanobody as used herein may refer to a single domain antibody derived from heavy-chain only (VHH) antibodies. In some embodiments, a nanobody includes any of monomeric, dimeric, bispecific or multivalent nanobodies that are specific for α-synuclein and are capable of inducing degradation of α-synuclein when part of the proteasomal degradation protein complex of the present invention.

The small size of nanobodies compared to conventional antibodies provides numerous advantages. Small polypeptide binders are ideal for intracellular expression as they do not require complex folding or disulphide bridge formation. The small size of a nanobody also allows access to hidden and/or grooved epitopes. Nanobodies are particularly useful in CNS applications due to their ability to cross the blood brain barrier.

In some embodiments of the present invention, nanobodies may be of camelid origin (e.g. camel, alpaca and llama) and/or shark origin. Nanobodies are non-endogenous proteins but are considered non-immunogenic or of low immunogenicity due to their high similarity with human variable heavy (VH) sequences. Suitably, therefore, the use of a nanobody as the polypeptide binder of the present invention may have the additional advantage of reducing immunogenicity of the complex.

### E3 Ubiquitin Ligase Components:

In embodiments as described herein, the E3 ubiquitin ligase component of the proteasomal degradation protein complex can be any E3 ubiquitin ligase component capable of recruiting and positioning the target protein proximal to the E3 ubiquitin ligase and its cognate E2-Ub conjugates. This facilitates the ubiquitylation and subsequent degradation of the target protein via the proteasome. Suitably, the proteasomal degradation protein complex targets α-synuclein protein for degradation by ubiquitin-mediated proteasomal degradation.

In some embodiments, the E3 ubiquitin ligase component is a Cullin ring E3 ligase (CRL) complex substrate receptor. Cullin ring ubiquitin ligases are multi-subunit E3 ubiquitin ligases which use a specific cullin as a central scaffold to bridge an E2 enzyme to the substrate. In some embodiments, the E3 ubiquitin ligase component is a substrate receptor which recruits α-synuclein to CUL2-CRL, for example the von-Hippel Lindau tumour suppressor (VHL) or a functional variant thereof. Suitably, the E3 ubiquitin ligase component is wild type VHL protein (SEQ ID NO: 13) or a functional variant thereof. Suitably a functional variant comprises a sequence that is at least 60% identical to wild type VHL (SEQ ID NO: 13), preferably at least 70, 80%, 90%, 95% or 99% identical to wild type VHL (SEQ ID NO: 13). A functional variant also comprises any variant of VHL that retains the capacity to recruit the CUL2-CRL machinery to the protein of interest.

In another embodiment, the E3 ubiquitin ligase component is wild type Kelch-like protein 6 (KLHL6) (SEQ ID NO: 15) or a functional variant thereof. Suitably a functional variant comprises a sequence that is at least 60% identical to wild type KLHL6 (SEQ ID NO: 15), preferably at least 70, 80%, 90%, 95% or 99% identical to wild type KLHL6 (SEQ ID NO: 15). A functional variant also comprises any variant of KLHL6 that retains the capacity to recruit CUL3 to the protein of interest.

In another embodiment, the E3 ubiquitin ligase component is wild type Kelch-like ECH-associated protein 1 (KEAP1) (SEQ ID NO: 16) or a functional variant thereof. Suitably a functional variant comprises a sequence that is at least 60% identical to wild type KEAP1 (SEQ ID NO: 16), preferably at least 70, 80%, 90%, 95% or 99% identical to wild type KEAP1. A functional variant also comprises any variant of KEAP1 that retains the capacity to recruit CUL3 to the protein of interest.

In another embodiment, the E3 ubiquitin ligase component is wild type Cereblon (CRBN) (SEQ ID NO: 17) or a functional variant thereof. Suitably a functional variant comprises a sequence that is at least 60% identical to wild type CRBN (SEQ ID NO: 17), preferably at least 70, 80%, 90%, 95% or 99% identical to wild type CRBN. A functional variant also comprises any variant of KEAP1 that retains the capacity to recruit CUL4 to the protein of interest.

In another embodiment, the E3 ubiquitin ligase component is wild type Kelch Domain Containing 2 (KLHDC2) (SEQ ID NO: 18) or a functional variant thereof. Suitably a functional variant comprises a sequence that is at least 60% identical to wild type KLHDC2 (SEQ ID NO: 18), preferably at least 70, 80%, 90%, 95% or 99% identical to wild type KLHDC2. A functional variant also comprises any variant of KEAP1 that retains the capacity to recruit CUL2 to the protein of interest.

In another embodiment, the E3 ubiquitin ligase component is wild type TRAF3d56 (SEQ ID NO: 19) or a functional variant thereof. Suitably a functional variant comprises a sequence that is at least 60% identical to wild type TRAF3d56 (SEQ ID NO: 19), preferably at least 70, 80%, 90%, 95% or 99% identical to wild type TRAF3d56. Without wishing to be bound by theory, it is thought that TRAF3d56 is a RING E3 ligase and may or may not require a CULLIN protein.

It should be noted that, while the present invention has been demonstrated in the specific examples below showing von-Hippel Lindau tumour suppressor (VHL) as the most potent E3 ubiquitin ligase component (i.e. VHL results in the greatest reduction in total cellular α-synuclein protein content), it will be apparent to the skilled person that other E3 ubiquitin ligase components can be used. This has been demonstrated in the Examples below where KLHL6, KEAP1, CRBN, KLHDC2 and TRAF3d56 are suitable E3 ligases for use in the present invention. The suitability of any putative E3 ubiquitin ligase for use in the present invention can readily be assessed by substituting the candidate E3 ubiquitin ligase for VHL in the examples described below. The skilled person will understand that in some embodiments, the total amount of α-synuclein in a cell degraded by the proteasomal degradation protein complex of the invention, may vary depending on the α-synuclein-specific polypeptide binder selected for use in the complex, the E3 ligase selected for use in the complex and/or the orientation of the component parts. Where it is desirable to degrade all or a significant proportion of the total cellular α-synuclein protein, it may be desirable to select VHL, KEAP1 or KLHL6 as the E3 ligase component. In an alternative embodiment, it may be desirable that a smaller proportion of the total cellular α-synuclein protein is degraded. In such embodiments, the skilled person may use any one of CRBN, KLHDC2 or TRAF3d56 as the E3 ligase component.

Humans have an estimated 500-1000 E3 ubiquitin ligases, which are classified into four families: HECT, RING-finger, U-box, and PHD-finger. Alternative E3 ubiquitin ligase components suitable for use in the present invention include, but are not limited to, the following (or functional variants thereof):

APPBP2, KLHDC10, KLHDC3, KLHDC2, LRR1, LRRC58, LRRC28, LRRC14, PRAME, VHL, LRRC42, MED8, RACK1, ARID1A, ARID1B, FEM1A, FEM1C, FEM1B, ZER1, ZYG11A, ZYG11B, ZSWIM5, ZSWIM8, ANKRD9, NEURL2, ASB1, ASB12, ASB11, ASB5, ASB9, ASB13, ASB7, ASB10, ASB18, ASB16, ASB4, ASB8, ASB14, ASB15, ASB2, ASB3, SPSB1, SPSB4, SPSB2, SPSB3, ASB17, ASB6, PCMTD1, PCMTD2, CISH, SOCS2, SOCS3, SOCS1, SOCS6, SOCS7, SOCS4, SOCS5, RAB40A, RAB40AL, RAB40B, RAB40C, WSB1, WSB2, ELOA, ELOA2, ELOA3, LRRC41, TULP4, BTRC, FBXW11, FBXW7, FBXW4, FBXW2, FBXW12, FBXW9, FBXW5, FBXW8, CDRT1, FBXW10, CCNF, ECT2L, FBXO16, FBXO36, FBXO43, FBXO5, FBXO17, FBXO27, FBXO2, FBXO44, FBXO6, NCCRP1, FBXO28, FBXO45, FBXO22, FBXO4, FBXO25, FBXO32, FBXO39, FBXO33, FBXO8, FBXO48, FBXO7, FBXO9, FBXO47, FBXO3, TSPAN17, FBXO15, FBXO31, FBXO24, FBXO21, FBXO34, FBXO46, FBXO30, FBXO40, FBXO42, FBXO41, FBXO10, FBXO11, FBXO18, FBXO38, LMO7, FBXL12, FBXL7, FBXL14, FBXL2, FBXL20, LRRC29, FBXL15, FBXL16, FBXL6, SKP2, FBXL13, FBXL17, FBXL4, FBXL21, FBXL3, FBXL8, FBXL22, FBXL19, KDM2A, KDM2B, FBXL5, FBXL18, DCAF8L2, DCAF8L1, DCAF8, WDTC1, DCAF6, DCAF5, CRBN, ERCC8, DDB2, RBBP7, RBBP4, GRWD1, NUP43, DCAF7, DCAF4L1, DCAF4, DCAF4L2, TLE3, TLE1, TLE2, TRPC4AP, TOR1AIP2, DCAF17, DCAF12L2, DCAF12L1, DCAF12, AHR, DCAF10, RBBP5, DCAF13, WDR53, WDR26, WDR61, SMU1, PAFAH1B1, NLE1, GNB2, WDR82, ATG16L1, SNRNP40, DCAF11, WDR5B, WDR5, POC1B, EED, WDR12, PWP1, DTL, KATNB1, CIAO1, DCAF1, DCAF16, DCAF15, WDR76, DET1, AMBRA1, WDR59, PHIP, BRWD1, KLHL4, KLHL1, KLHL5, KLHL8, KLHL20, KLHL3, KLHL2, KLHL17, KLHL18, KLHL12, KLHL7, IVNS1ABP, KLHL28, KLHL10, KEAP1, IPP, KLHL23, KLHL34, BCL6B, KLHL32, KLHL15, KLHL36, KLHL22, KLHL9, KLHL13, KLHL26, KLHL14, KLHL31, KLHL42, KBTBD11, KBTBD13, KLHL33, GAN, KLHL35, KLHL24, KLHL6, KLHL29, KLHL38, KLHL25, ENC1, CCIN, KLHL11, KBTBD4, KBTBD3, KLHL30, KLHL21, KBTBD7, KBTBD6, KBTBD8, KBTBD2, KBTBD12, KLHL41, KLHL40, BTBD6, BTBD3, BTBD2, BTBD1, TNFAIP1, KCTD13, KCTD10, KCTD7, KCTD14, KCTD8, KCTD12, KCTD16, KCTD18, KCTD15, KCTD1, KCTD6, KCTD4, KCTD21, SHKBP1, KCTD3, KCTD5, KCTD17, KCTD2, KCTD9, KCNRG, KCTD19, KCTD11, ARMC5, KCTD20, BTBD10, SLX4, RCBTB2, RCBTB1, IBTK, SPOPL, SPOP, LZTR1, RHOBTB2, RHOBTB1, RHOBTB3, ABTB2, ABTB1, CDC20, FZR1, Anaphase-promoting complex (APC), BC-box, eloBC, CUL5, RING, LNXp80, CBX4, PIAS1, PIAS2, PIAS3, PIAS4 or RANBP2.

Alternative E3 ubiquitin ligase components may include single polypeptide E3 ligases. Single polypeptide E3 ligases suitable for use in the present invention include, but are not limited to, the following (or functional variants thereof):

UBR1, UBR2, UBR7, UBR3, UBR4, NOSIP, PPIL2, STUB1, PRPF19, WDSUB1, UBE4A, UBE4B, UBOX5, MEFV, TRIM10, TRIM15, TRIM26, TRIM31, TRIM11, TRIM58, TRIM21, TRIM68, TRIM38, TRIML1, TRIM17, TRIM27, TRIM7, TRIM39, TRIM60, TRIM61, TRIM4, TRIM62, TRIM69, TRIM22, TRIM34, TRIM6, TRIM5, TRIM43, TRIM48, TRIM49D1, TRIM49, TRIM49B, TRIM51, TRIM64, TRIM77, TRIM35, TRIM50, TRIM73, TRIM74, TRIM72, TRIM41, TRIM52, TRIM40, TRIM13, TRIM59, TRIM65, TRIM14, TRIM25, TRIM8, TRIM16, TRIM47, TRIM29, MID1, MID2, TRIM44, TRIM54, TRIM55, TRIM63, TRIM67, TRIM9, TRIM36, TRIM46, TRIM2, TRIM3, TRIM71, TRIM45, TRIM56, TRIM32, TRIM24, TRIM33, TRIM28, TRIM23, TRIM42, PML, TRIM37, TRAF2, TRAF3, TRAF5, TRAF4, TRAF6, TRAF7, TRAF3d56, ANKIB1, ARIH1, ARIH2, RNF144A, RNF144B, RNF217, RNF14, PRKN, RBCK1, RNF19A, RNF19B, RNF216, RNF31, NEURL3, NEURL1B, NEURL1, RNF34, RFFL, MUL1, CGRRF1, UNKL, UNK, RNF123, VPS8, VPS18, MIB1, MYLIP, DCST1, RNF220, LTN1, RNF214, RAPSN, RNF32, RNF213, RNF26, BRAP, NSMCE1, VPS11, CBLB, CBL, CBLC, RNF113B, RNF113A, RNF8, RNF208, PHF7, LRSAM1, FANCL, TMEM129, LONRF2, LONRF1, PEX10, RNF139, RNFT1, PJA2, PJA1, RNF4, LNX2, LNX1, TTC3, RNF157, MGRN1, PELI2, PELI1, PELI3, RNF180, RNF181, RNF175, RNF121, RNF24, RNF122, DZIP3, PEX2, RNF150, RNF130, RNF149, RNF148, RNF133, RNF128, RNF167, RNF13, ZNRF4, RNF44, RNF38, RNF6, RLIM, ZNRF2, ZNRF1, RNF11, RNF103, RNF145, RNF126, RNF115, RNF141, SHPRH, RNF186, LONRF3, ZNRF3, RNF43, RNF215, RNF165, RNF111, RFWD3, PEX12, ANAPC11, RNF112, RNF187, RNF169, RNF168, RNF40, RNF20, RNF219, CCNB1IP1, RFPL3, RFPL1, RFPL4A, RFPL4B, RNF39, PDZRN4, PDZRN3, RNF41, BRCA1, BARD1, RC3H2, RC3H1, SYVN1, AMFR, UHRF2, UHRF1, RNF17, RSPRY1, RNF135, NHLRC1, TOPORS, RNF207, RNF183, SIAH2, SIAH1, IRF2BPL, MAP3K1, CNOT4, RNF166, ZNF598, GID4, SH3RF2, RNF146, RNF151, SH3RF1, RNF182, RNF152, TRAIP, RNF10, RAG1, BFAR, MIB2, RNF25, RNF138, RNF114, RNF125, HLTF, RNF170, RNF5, RNF185, MNAT1, COP1, PCGF5, PCGF3, PCGF6, PCGF2, BMI1, PCGF1, RNF2, RING1, PHRF1, CHFR, ZSWIM2, MSL2, RNF212, RBBP6, RAD18, ZFPL1, MYCBP2, VPS41, CBLL2, CBLL1, RCHY1, RNF7, RBX1, MEX3A, MEX3B, MEX3C, MEX3D, MDM2, MDM4, MARCH1, MARCH8, MARCH2, MARCH3, MARCH11, MARCH4, MARCH9, MARCH5, MARCH10, MARCH7, MARCH6, MKRN1, MKRN3, MKRN2, AREL1, HECTD2, UBE3A, HERC3, HERC4, HERC5, HERC6, HACE1, HECW1, HECW2, ITCH, WWP1, WWP2, SMURF1, SMURF2, NEDD4, NEDD4L, HUWE1, UBE3D, G2E3, HECTD3, UBE3B, UBE3C, TRIP12, HECTD1, UBR5, HECTD4, HERC1, HERC2, DTX1, DTX4, DTX2, DTX3, DTX3L, BIRC2, BIRC3, BIRC8, XIAP or BIRC7.

In some embodiments, the E3 ubiquitin ligase component of the proteasomal degradation protein complex is an E3 ubiquitin ligase component that is functional in the nervous system suitably the peripheral nervous system or the central nervous system. In a preferred embodiment, the E3 ubiquitin ligase component of the proteasomal degradation protein complex is an E3 ubiquitin ligase component that is functional in the central nervous system.

Suitably, in some embodiments the E3 ubiquitin ligase is a HECT type ligase including the Nedd4 family, HERC family, and other HECT type ligases. In some embodiments the E3 ubiquitin ligase is RNF183.

Without wishing to be bound by theory, any α-synuclein-specific polypeptide binder and any E3 ubiquitin ligase components that selectively bind and correctly position α-synuclein for ubiquitination are suitable for use in the present invention. Suitable combinations of α-synuclein-specific polypeptide binders and E3 ubiquitin ligase components can be readily identified using the methodologies described herein.

The skilled person will understand that in some embodiments the orientation of the α-synuclein-specific polypeptide binder and any E3 ubiquitin ligase components of the proteasomal degradation protein complex may affect the activity or potency of the complex. Suitably, in some embodiments, the α-synuclein-specific polypeptide binder is positioned at the N-terminus of the protein complex. Alternatively, in some embodiments, the α-synuclein-specific polypeptide binder is positioned at the C-terminus of the protein complex. In some embodiments, the E3 ubiquitin ligase component is positioned at the N-terminus of the protein complex. In an alternative embodiment, the E3 ubiquitin ligase component is positioned at the C-terminus of the protein complex. The optimum orientation can be readily identified using the methodologies described herein.

It will be understood that the component parts of proteasomal degradation protein complex of the present invention may be tethered or interlinked via a linker protein. Suitable linkers are known in the art and are described in the examples below. In one embodiment, a linker protein comprises the amino acid sequence 5′-GGGGG-3′ (SEQ ID NO: 28). In an alternative embodiment, the E3 ligase component as described herein and the α-synuclein-specific polypeptide binder may be provided as individual components that conjugate when expressed, e.g. in a cell. Suitably, in such embodiments, the E3 ligase component and the α-synuclein-specific polypeptide binder are modified or tagged with a suitable protein conjugation system known in the art. Suitably, the proteasomal degradation protein complex may be provided as a protein complex via an interaction of a domain and a binding partner thereof. For example, in one embodiment, the E3 ligase component may be biotinylated and the α-synuclein-specific polypeptide binder may have a streptavidin tag, or vice versa. Alternatively, the E3 ligase component may be streptavidin tagged and the α-synuclein-specific polypeptide binder is biotinylated. Other protein binding pairs are known in the art (e.g. SH3 domain, PDZ domain, GK domain, GB domain and the like and a binding partner thereof). In yet another embodiment, inteins can be provided on the components of the complex to fuse an E3 ligase component to a α-synuclein-specific polypeptide binder to form the complex.

### Nucleic Acids and Vectors:

In a second aspect of the present invention, there is provided one or more nucleic acid constructs encoding a proteasomal degradation protein complex of the first aspect.

In some embodiments, the nucleic acid construct comprises a nucleic acid encoding an E3 ubiquitin ligase component linked to a nucleic acid encoding an α-synuclein-specific polypeptide binder.

It will be apparent to the skilled person that in some embodiments, e.g. where the complex is not a fusion protein, the individual components of the complex can be provided on separate nucleic acid constructs, and reference to a nucleic acid construct herein should be read accordingly (i.e. not to exclude the possibility of there being more than one nucleic acid construct, where this is appropriate). Thus, in some embodiments, one or more expression constructs may be provided which comprise nucleic acid sequences encoding the separate components of the complex. In some embodiments, a first nucleic acid construct comprises a first nucleic acid encoding an E3 ubiquitin ligase component and a second nucleic acid construct comprises a second nucleic acid encoding an α-synuclein-specific polypeptide binder. Suitably, in such embodiments, the first and second nucleic acids encode the E3 ligase component and α-synuclein-specific polypeptide binder components linked to suitable elements to allow the components to conjugate and form the active complex.

In some embodiments, the nucleic acid construct comprises a nucleic acid encoding VHL or a functional variant thereof linked to a nucleic acid encoding an α-synuclein-specific polypeptide binder. Suitably, in some embodiments the α-synuclein-specific polypeptide binder is a nanobody, and in some embodiments the α-synuclein-specific polypeptide binder may be NbSYN87 or a functional variant or biological equivalent thereof.

In one embodiment, the nucleic acid construct comprises a nucleic acid encoding VHL according to SEQ ID NO: 2 or a functional variant thereof, and a nucleic acid encoding the nanobody NbSYN87 according to SEQ ID NO: 1 or a functional variant thereof.

In some embodiments, the nucleic acid construct comprises a nucleic acid sequence according to SEQ ID NO: 3 or a functional variant thereof. Suitably, in some embodiments, the nucleic acid construct comprises a nucleic acid sequence that is at least 60, 70%, 80%, 90%, 95% or 99% identical to SEQ ID NO: 3.

In one embodiment, the nucleic acid construct comprises a nucleic acid encoding KLHL6 according to SEQ ID NO: 6 or a functional variant thereof, and a nucleic acid encoding the nanobody NbSYN87 according to SEQ ID NO: 1 or a functional variant thereof.

In one embodiment, the nucleic acid construct comprises a nucleic acid encoding KEAP1 according to SEQ ID NO:7 or a functional variant thereof, and a nucleic acid encoding the nanobody NbSYN87 according to SEQ ID NO: 1 or a functional variant thereof.

In one embodiment, the nucleic acid construct comprises a nucleic acid encoding CRBN according to SEQ ID NO: 8 or a functional variant thereof, and a nucleic acid encoding the nanobody NbSYN87 according to SEQ ID NO: 1 or a functional variant thereof.

In one embodiment, the nucleic acid construct comprises a nucleic acid encoding KLHDC2 according to SEQ ID NO: 9 or a functional variant thereof, and a nucleic acid encoding the nanobody NbSYN87 according to SEQ ID NO: 1 or a functional variant thereof.

In one embodiment, the nucleic acid construct comprises a nucleic acid encoding TRAF3d56 according to SEQ ID NO: 10 or a functional variant thereof, and a nucleic acid encoding the nanobody NbSYN87 according to SEQ ID NO: 1 or a functional variant thereof.

Nucleic acids which encode a proteasomal degradation protein complex of the invention may be wholly or partially synthetic and may include, but are not limited to, DNA, cDNA and RNA. Nucleic acid sequences encoding the proteasomal degradation protein complex of the invention can be readily prepared by the skilled person using techniques which are well known to those skilled in the art, such as those described in Sambrook et al. “Molecular Cloning”, A laboratory manual, Cold Spring Harbor Laboratory Press, Volumes 1-3, 2001 (ISBN-0879695773), and Ausubel et al. Short Protocols in Molecular Biology. John Wiley and Sons, 4th Edition, 1999 (ISBN-0471250929). Said techniques include (i) the use of the polymerase chain reaction (PCR) to amplify samples of nucleic acid, (ii) chemical synthesis, or (iii) preparation of cDNA sequences. DNA encoding proteasomal degradation protein complex of the invention may be generated and used in any suitable way known to those skilled in the art, including taking encoding DNA, identifying suitable restriction enzyme recognition sites either side of the portion to be expressed, and cutting out said portion from the DNA. The excised portion may then be operably linked to a suitable promoter and expressed in a suitable expression system, such as a commercially available expression system. Alternatively, the relevant portions of DNA can be amplified by using suitable PCR primers. Modifications to the DNA sequences can be made by using site directed mutagenesis.

Nucleic acid sequences encoding a proteasomal degradation protein complex of the invention may be provided as expression constructs in the form of a plasmid, vector, transcription or expression cassette which comprises at least one nucleic acid as described above operably liked to one or more expression control sequences, e.g. a promoter, an enhancer, a poly-A sequence, an intron or suchlike. Suitably the expression control sequences are sufficient to provide expression of the proteasomal degradation protein complex in a target cell. The expression may be constitutive or regulatable.

Accordingly, in a third aspect of the present invention, there is provided an expression construct comprising a nucleic acid construct as set out above. Suitably the expression construct is a vector, e.g. an expression vector adapted for expression in a eukaryotic or prokaryotic cell.

Suitably, in some embodiments of the present invention the vector is a viral vector, such as a retroviral, lentiviral, adenoviral, or adeno-associated viral (AAV) vector. In some preferred embodiments the vector is an AAV vector.

In some embodiments, the vector is a gene therapy vector, suitably an AAV vector, an adenoviral vector, a retroviral vector, a herpes simplex vector or a lentiviral vector. Lentiviral vectors have been extensively used as a gene transfer tool in the CNS and are known to be able to successfully transduce neurones, astrocytes and oligodendrocytes. They are beneficial as they have relatively large cloning capacity and because viral genes are not expressed. A particularly preferred lentiviral vector system is based on HIV-1. Herpes simplex viral vectors and adenoviral vectors also show potential for use in as a gene transfer tool in CNS as they show successful transduction of CNS cells but are less preferred as due to their toxicity.

AAV vectors have been extensively discussed in the art. AAV vectors are of particular interest as AAV vectors do not typically integrate into the genome and do not elicit immune response. AAV serotypes 1, 2, 4, 5, 8, 9 and 2g9 (AAV1, AAV2, AAV4, AAV5, AAV8, AAV9 and AAV2g9) have been noted to achieve efficient transduction in the CNS. Therefore, AAV1, AAV2, AAV4, AAV5, AAV8, AAV9 and derivatives thereof are particularly preferred AAV serotypes. In some embodiments, AAV9 is particularly preferred AAV vector. In other embodiments, AAV2g9 is a particularly preferred AAV vector (WO2014/144229). In yet other embodiments, a particularly preferred AAV vector is AAVDJ8 (Hammond et al., 2017). Suitably an AAV vector comprises a viral genome which comprises a nucleic acid sequence of the present invention positioned between two inverted terminal repeats (ITRs). WO2019/028306, for example discloses various wild type and modified AAV vectors that can be used in the CNS. In one embodiment, the AAV vector is capable of penetrating the blood brain barrier following delivery of the AAV vector. In one embodiment, AAV vectors of the present invention are recombinant AAV viral vectors which are replication defective, lacking sequences encoding functional Rep and Cap proteins within their viral genome. These defective AAV vectors may lack most or all parental coding sequences and essentially carry only one or two AAV ITR sequences and the nucleic acid of interest for delivery to a cell, a tissue, an organ or an organism. Suitably AAV vectors for use herein comprise a virus that has been reduced to the minimum components necessary for transduction of a nucleic acid payload or cargo of interest. In this manner, AAV vectors are engineered as vehicles for specific delivery while lacking the deleterious replication and/or integration features found in wild-type viruses. In one embodiment, the AAV particle of the present invention is an scAAV. In another embodiment, the AAV particle of the present invention is an ssAAV. Methods for producing and/or modifying AAV particles are disclosed extensively in the art (see e.g. WO2000/28004; WO02001/23001; WO2004/112727; WO 2005/005610 and WO 2005/072364, which are incorporated herein by reference). In one embodiment the AAV vector comprises a capsid that allows for blood brain barrier penetration following intravascular (e.g. intravenous or intraarterial) administration (see e.g. WO2014/144229, which discusses, for example, capsids engineered for efficient crossing of the blood brain barrier, e.g. capsids or peptide inserts including VOY101, VOY201, AAVPHP.N, AAVPHP.A, AAVPHP.B, PHP.B2, PHP.B3, G2A3, G2B4, G2B5, PHP.S, and variants thereof).

Methods of making AAV vectors are well known in the art and are described in e.g., U.S. Pat. Nos. 6,204,059, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508, 5,064,764, 6,194,191, 6,566,118, 8,137,948; or International Publication Nos. WO1996039530, WO1998010088, WO 1999014354, WO1999/015685, WO1999/047691, WO2000/055342, WO2000/075353 and WO2001/023597; Methods In Molecular Biology, ed. Richard, Humana Press, NJ (1995); O'Reilly et al, Baculovirus Expression Vectors, A Laboratory Manual, Oxford Univ. Press (1994); Samulski et al., J Fir. 63:3822-8 (1989); Kajigaya et al, Proc. Nat'l. Acad. Sci. USA 88: 4646-50 (1991); Ruffing et al., J. Vir. 66:6922-30 (1992); Kimbauer et al, Vir., 219:37-44 (1996); Zhao et al, Vir. 272: 382-93 (2000); the contents of each of which are herein incorporated by reference. Viral replication cells commonly used for production of recombinant AAV viral particles include but are not limited to HEK293 cells, COS cells, HeLa cells, KB cells, and other mammalian cell lines.

In some embodiments the vector is a non-viral vector, for example using cationic polymers or cationic lipids, as is known in the art. Various non-viral vectors are discussed in Selene Ingusci et al. (Gene Therapy Tools for Brain Diseases. Front. Pharmacol. 10:724. doi: 10.3389)

In some embodiments, there is provided a virion (viral particle) comprising a vector, suitably a viral vector, according to the present invention. In some embodiments the virion is an AAV virion.

The invention thus further provides recombinant virions (viral particles) comprising a vector as described above.

### Pharmaceutical Compositions:

In another aspect the present invention provides a pharmaceutical composition comprising a therapeutically effective amount of a proteasomal degradation protein complex as set out above. Such a composition typically comprises at least one pharmaceutically acceptable diluent or carrier.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host. Suitable carriers may be readily selected by one of skill in the art in view of the indication for which the pharmaceutical composition is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure. Various other conventional pharmaceutical ingredients may be provided in the pharmaceutical composition, such as preservatives or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

Delivery vehicles such as liposomes, nanocapsules, microparticles, microspheres, lipid particles, vesicles, and the like, may be used for the introduction of the compositions of the present disclosure into suitable host cells. In particular, the vector delivered transgenes may be formulated for delivery either encapsulated in a lipid particle, a liposome, a vesicle, a nanosphere, or a nanoparticle or the like.

Suitably in one aspect of the invention, there is provided a pharmaceutical composition comprising the proteasomal degradation protein complex, nucleic acid, expression construct, vector, or virion as discussed above and a pharmaceutically acceptable carrier or diluent. Suitably, in one embodiment, the composition is suitable to act as an inhibitor of α-synuclein in target cells. In a further embodiment, the composition is suitable to regulate levels of α-synuclein in target cells. Regulating protein level may refer to reducing or increasing the level of α-synuclein. Suitably, the pharmaceutical composition results in the degradation of α-synuclein.

Suitably, in one embodiment the proteasomal degradation protein complex or the pharmaceutical composition according to any of the aspects and embodiments provided herein, targets α-synuclein for proteasomal degradation in a target cell. Optionally, the α-synuclein has a SNCA duplication, triplication and/or any point mutation selected from the group comprising: A53T, A30P, H50Q, E46K, G51D and/or A53E. Suitably, in some embodiments α-synuclein is monomeric, oligomeric, protofibrillar, mature fibrils or aggregated.

Suitable target cells include any eukaryotic cell. Preferably, the target cell is mammalian, more preferably human. In some embodiments the target cell is a neuronal cell, preferably a cell from the central nervous system. The neuronal cell may be a primary neuronal cell or a cell of a neurone derived cell line, e.g. an immortalised cell line. In some embodiments, the target cell is a neurone, astrocytes, oligodendrocytes, microglial cells and/or ependymal cells.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

### Therapy and Related Methods:

In a further aspect the present invention provides a method of treatment or prevention of a disease in a subject in need thereof, the method comprising administering to said subject a therapeutically effective amount of the proteasomal degradation protein complex, expression construct, vector, virion or pharmaceutical composition as discussed above.

Suitably the method comprises introducing into cells of the subject a proteasomal degradation protein complex, expression construct, vector, virion or pharmaceutical composition as discussed above. Suitable target cells are discussed above.

In some embodiments the method comprises administering a vector or virion according to the present invention to the subject. Suitably the vector is a viral gene therapy vector, for example an AAV vector.

The present invention also provides a proteasomal degradation protein complex, expression construct, vector, virion or pharmaceutical composition as described herein for use in a method of treatment or prevention of a disease in a subject. The method suitably comprises administering to said subject a therapeutically effective amount of a proteasomal degradation protein complex or pharmaceutical composition of the present invention.

Suitably the proteasomal degradation protein complex, expression construct, vector, virion or pharmaceutical composition as discussed above is used for the treatment, prophylaxis, palliation or amelioration of a neurological disease and/or disorder. In one embodiment, the proteasomal degradation protein complex, expression construct, vector, virion or pharmaceutical composition is for use in the treatment of a subject with a neurodegenerative disorder. In some embodiments, the neurodegenerative disorder is a synucleinopathy. In some embodiments, the neurodegenerative disorder is any of PD, dementia and/or multiple system atrophy.

In some embodiments, the method comprises administering a proteasomal degradation protein complex, expression construct, vector, virion or pharmaceutical composition systemically. Systemic administration may be enteral (e.g. oral, sublingual, and rectal) or parenteral (e.g. injection). Suitable methods of administration may be enteral (e.g. oral, sublingual, and rectal) or parenteral (e.g. injection) including intravenous, intraarterial, intracranial, intramuscular, subcutaneous, intra-articular, intrathecal, and intradermal injections. Preferred administration methods are intravenous, intraarterial, intracranial and intrathecal injection.

In some embodiments the method comprises introducing into the CNS of the subject a pharmaceutical composition as described herein. A particular difficulty with introducing a vector, virion or a pharmaceutical composition in the CNS is the blood brain barrier. The blood brain barrier is a semipermeable border of endothelial cells that prevents certain chemicals and molecules in the bloodstream from crossing into the extracellular fluid of the central nervous system. In animal studies, this obstacle has been overcome by injection directly into the brain of the animal, such as intracranial injection, suitably intracerebroventricular (ICV) injection (see e.g. Keiser et al., Curr Protoc Mouse Biol. 2018 December; 8(4):e57). This method of administration can be disadvantageous for gene therapy in humans as it is difficult to perform and can be dangerous for the subject.

Instead, in a gene therapy setting in human, it is preferred that the expression cassette as described herein is introduced into the CNS by intravenous or intraarterial (e.g. intra-carotid) administration of a viral vector comprising the expression cassette. Suitably, the viral vector is an AAV vector. Intravenous or intraarterial administration of some serotypes of AAV allows penetration of the AAV vectors into the brain. Intravenous or intraarterial administration is safer and less invasive than intracranial administration, while still allowing penetration through the blood brain barrier.

In some embodiments, a viral gene therapy vector may be administered concurrently or sequentially with one or more additional therapeutic agents or with one or more saturating agents designed to prevent clearance of the vectors by the reticular endothelial system.

Where the vector is an AAV vector, the dosage of the vector may be from 1×10¹⁰ gc/kg to 1×10¹⁵ gc/kg or more, suitably from 1×10¹² gc/kg to 1×10¹⁴ gc/kg, suitably from 5×10¹² gc/kg to 5×1013 gc/kg.

In general, the subject in need of treatment will be a mammal, and preferably primate, more preferably a human. Typically, the subject in need thereof will display symptoms characteristic of a disease, e.g. a disease discussed above, most preferably a synucleinopathy. The method typically comprises ameliorating the symptoms displayed by the subject in need thereof, by expressing the therapeutic amount of the therapeutic product of the invention.

Gene therapy protocols for therapeutic gene expression in target cells in vitro and in vivo, are well-known in the art and will not be discussed in detail here. Briefly, they include intramuscular injection, interstitial injection, instillation in airways, application to endothelium, intra-hepatic parenchyme, and intravenous or intra-arterial administration (e.g. intra-hepatic artery, intra-hepatic vein) of plasmid DNA vectors (naked or in liposomes) or viral vectors. Various devices have been developed for enhancing the availability of DNA to the target cell. While a simple approach is to contact the target cell physically with catheters or implantable materials containing the relevant vector, more complex approaches can use jet injection devices and suchlike. Gene transfer into mammalian cells has been performed using both ex vivo and in vivo procedures. The ex vivo approach typically requires harvesting of the cells, in vitro transduction with suitable expression vectors, followed by reintroduction of the transduced hepatocytes the liver. In vivo gene transfer has been achieved by injecting DNA or viral vectors.

In a further aspect of the invention there is provided a proteasomal degradation protein complex, expression construct, vector, virion or pharmaceutical composition according to any aspect of the invention for use as medicament, e.g. for treatment of a patient. Suitably the patient is suffering from a synucleinopathy.

α-synuclein:

α-synuclein is a member of the intrinsically disordered protein (IDP) family, α-synuclein itself has no tertiary structure and its conformation can be affected by a number of different factors such as the presence of interactors or lipid membranes (15). Its role in PD is further complicated by the fact that the six different mutations of the protein found in patients with familial PD have also been shown to prefer differential oligomeric and fibrillar forms during the aggregation process (16). Interestingly, α-synuclein within these Lewy bodies is nearly all phosphorylated at serine 129 (17) which can be used as a marker to confirm the presence of these aggregated structures as only trace levels of the phosphorylated protein are present in the brains of healthy individuals compared to patients with PD. It is unclear whether this phosphorylation promotes aggregation of the protein or whether it happens after aggregate formation. Whether the toxicity elicited on cells is due to the aggregates themselves, via a toxic gain of function, or due to recruitment of a synuclein from its endogenous site of action, eliciting a loss of function, is also yet to be determined.

Suitably, in one embodiment the proteasomal degradation protein complex according to any aspect provided herein targets α-synuclein for proteasomal degradation. Suitably, wherein α-synuclein has a SNCA duplication, triplication and/or any point mutation selected from the group comprising: A53T, A30P, H50Q, E46K, G51D and/or A53E. α-synuclein targeted for degradation according to any aspect of the present invention is monomeric, oligomeric, protofibrillar, mature fibrils or aggregated.

In a further aspect, the invention provides a method for targeting α-synuclein for degradation using the proteasomal degradation protein complex of any aspect described herein.

In one embodiment, the method comprises:
administering proteasomal degradation protein complex, expression construct, vector, virion or pharmaceutical composition of the present invention to a cell, which may be in vitro or in vivo;
wherein the α-synuclein-specific polypeptide binder component of the proteasomal degradation protein complex binds to α-synuclein;
the E3 ligase component tethered to the α-synuclein-specific polypeptide binder recruits the α-synuclein protein to E3 ligase system in the cell;
the E3 ligase system in the cell ubiquitinylates α-synuclein such that α-synuclein is degraded.

The skilled person will understand that the proteasomal degradation protein complex E3 ubiquitin ligase component will dictate which Cullin protein is recruited to the complex. E3 ubiquitin ligases each use a specific Cullin as a central scaffold when recruiting E2 ligases. Suitably, in some embodiments the E3 ligase component of the present invention will recruit it's cognate Cullin e.g. any one of CULs 1, 2, 3, 4A, 4B, 5, or 7. Suitably, in some embodiments, the E3 ligase system comprises CUL2-CRL. In another embodiment, the E3 ligase system comprises CUL3. In yet another embodiment, the E3 ligase system comprises CUL4A. In another embodiment, the E3 ligase system comprises CUL4B. In some embodiments, the E3 ligase system comprises CUL5. In another embodiment, the E3 ligase system comprises CUL7. 6. Suitably, in one embodiment the proteasomal degradation protein complex of the invention, the E3 ubiquitin ligase component is capable of recruiting α-synuclein to any of CUL2-CRL, CUL3 or CUL4. Suitably, in embodiments where the E3 ligase component is TRAF3d56, i.e. a variant of TRAF3, it may be that no Cullin protein is recruited to the complex.

In one embodiment the proteasomal degradation protein complex, expression construct, vector, virion or pharmaceutical composition is administered to a cell. Suitably the cell is any target cell as described above.

In a preferred embodiment, α-synuclein is targeted for proteasomal degradation and degraded via ubiquitin-mediated proteasomal degradation. In some embodiments described herein, α-synuclein targeted for degradation comprises a SNCA duplication, triplication and/or any point mutation selected from the group comprising: A53T, A30P, H50Q, E46K, G51D and/or A53E.

Suitably, in some embodiments α-synuclein is monomeric, oligomeric, protofibrillar, mature fibrils or aggregated.

### Research Tools:

In a further aspect of the present invention, the proteasomal degradation protein complex of the present invention is used as a tool to investigate pathways of interest in a cell. Suitably, the proteasomal degradation protein complex is applicable as a research tool. In some embodiments the proteasomal degradation protein complex comprises an E3 ligase tethered to a polypeptide binder specific for a protein of interest (POI). Suitably, the proteasomal degradation protein complex degrades the POI. In some embodiments, the proteasomal degradation protein complex controls the expression of the POI. Suitably, the proteasomal degradation protein complex is delivered to a cell, preferably a mammalian cell. In some embodiments the cell is a central nervous system (CNS) cell. In some embodiments, the POI is α-synuclein.

In some embodiments, where the proteasomal degradation protein complex of the present invention is used as a research tool, the proteasomal degradation protein complex can be delivered to a cell in vitro, ex vivo or in vivo.

### Kits:

In one aspect, there is provided a kit for use in any of the aspects and embodiments of the present invention, wherein the kit comprises proteasomal degradation protein complex, expression construct, vector, virion or pharmaceutical composition as discussed above, and instructions for use.

In any aspect and/or embodiment as described herein, the proteasomal degradation protein complex is suitably an affinity-directed protein missile (AdPROM).

### BRIEF DESCRIPTION OF THE FIGURES

FIG. 1: Schematic depicting the AdPROM system using NbSYN87 nanobody tethered to VHL to induce polyubiquitination of α-synuclein.

FIG. 2: (A) VHL tethered to a GFP specific nanobody degrades α-synuclein. No α-synuclein degradation was observed using RIM32, RNF126, SIAH3, RNF144A, RNF125 E3 ligases. (B) U2OS Flp-In-T-Rex GFP-alpha-synuclein cells were retrovirally transduced to express the Ubch5a, WDR5, KLHDC2, MDM2, KLHL6, KLHL7, TRAF3d52, TRAF3d56, TRAF4, LONRF2 (P430-N500), LONRF2 (339-754), TRAF3, WWP1, WWP2, VHL, TRIM24, KEAP1, PHIP, and CRBN E3 ligase constructs and associated negative controls. (C) U20S Flp-In T-Rex GFP-alpha-synuclein cells were treated with the NEDDylation inhibitor MLN4924, the proteasomal inhibitors MG-132 or bortezomib, the lysosomal inhibitor bafilomycin-A1 or DMSO as a negative control for 14 hr with the indicated concentrations before cell lysis. U20S Flp-In T-Rex GFP-alpha-synuclein cells expressing either FLAG-KLHL6-aGFP16 or FLAG-aGFP16-KLHL6. U20S FIp-In T-Rex GFP-alpha-synuclein cells expressing either FLAG-VHL-aGFP16 or FLAG-KEAP1-aGFP16. U20S FIp-In T-Rex GFP-alpha-synuclein FLAG-CRBN-aGFP16 or FLAG-TRAF3d56-aGFP16 expressing cell lines.

FIG. 3: Targeted degradation of GFP-α-synuclein through the use of both an anti-GFP nanobody and an anti-α-synuclein nanobody with the AdPROM system. A) U2OS Flp-In T-REX cells expressing GFP tagged α-synuclein, either wildtype or an A53T mutant, under the control of the Tet On promoter were treated with 20 ng/ml doxycycline for the indicated time points. Cells were lysed with 20 μg of protein being resolved by SDS-PAGE, transferred onto a nitrocellulose membrane and immunoblotted with the indicated antibodies. U20S FIp-In T-Rex cells expressing GFP-tagged α-synuclein were retrovirally infected to express the VHL-aGFP AdPROM construct or the VHL and aGFP alone controls. Cells were lysed with 20 μg of lysate being resolved by SDS-PAGE, transferred onto nitrocellulose membranes and immunoblotted with the indicated antibodies. B) U20S FIp-In T-Rex cells expressing GFP-tagged wildtype α-synuclein were retrovirally infected to express either the VHL-aGFP AdPROM construct or VHL conjugated to one of two α-synuclein-specific nanobodies (NbSYN87 and NbSYN2). Cells were lysed with 20 μg of lysate being resolved by SDS-PAGE, transferred onto nitrocellulose membranes and immunoblotted with the indicated antibodies. C) As in B but with cells expressing the A53Tmutant of GFP-tagged α-synuclein.

FIG. 4: Targeted degradation of untagged α-synuclein through the AdPROM system. A) Schematic depicting the use of NbSYN87 tethered to VHL to induce polyubiquitination of untagged α-synuclein through the AdPROM system. B) U2OS FIp-In T-Rex cells expressing either wildtype of an A53T mutant of α-synuclein were retrovirally infected to express either VHL-NbSYN87 or VHL-aGFP alongside the appropriate controls. Cells were lysed with 20 μg of lysate being resolved by SDS-PAGE, transferred onto nitrocellulose membranes and immunoblotted with the indicated antibodies. C) HeLa Flp-In T-Rex cells expressing either wildtype or A53T α-synuclein were retrovirally infected to express VHL tethered to either NbSYN2 or NbSYN87 along with the appropriate controls. Cells were lysed and 20 μg of lysate was resolved by SDS-PAGE. D) Schematic depicting the three main regions of α-synuclein showing the 5 familial mutants within the N terminal amphiphatic region. The binding site of the nanobody NbSYN87 in the C-terminal domain is depicted by the red line. E) HeLa Flp-In T-Rex cells expressing either A30P or E46K mutants of α-synuclein were retrovirally infected to express VHL-NbSYN87 along with VHL and NbSYN87 controls. Cells were lysed with 20 μg of lysate being resolved by SDS-PAGE. F) As in E but with G51D and H50Q mutants of α-synuclein.

FIG. 5: Interaction between NbSyn87 and endogenous alpha-synuclein: A) SKMEL13 WT (infected with Flag-VHL or 3XFlag-NbSYN87 (as indicated) or KO cell extracts (input), endogenous alpha-synuclein immunoprecipitates from these extracts (IP) or post-IP extracts (Flowthrough) were subjected to Western blot analysis with the indicated antibodies. B) SK-MEL13 cells transiently transfected with control vector (Empty) or one encoding GFP-NBSYN87 were processed for IF and analysed by fluorescence microscopy for co-localisation of GFP-NbSyn87 and endogenous alpha-synuclein. Representative images are attached. DAPI staining was performed for nuclear DNA staining.

FIG. 6: Targeted degradation of endogenous α-synuclein in melanoma cells. A) A panel of five different melanoma cells were lysed and 20 μg of lysate was resolved by SDS-PAGE and immunoblotted for α-synuclein. B) SK-MEL13 and G-361 cells were retrovirally infected to express either VHL-NbSYN87 or VHL-aGFP alongside the appropriate controls. Cells were lysed with 20 μg of lysate being resolved by SDS-PAGE. C) G-361 cells expressing either VHL-NbSYN87 or NbSYN87 alone were treated with 1 uM of the NEDDylation inhibitor MLN4924 for 24 hours before being lysed. 20 μg of lysate was resolved by SDS-PAGE.

FIG. 7: SK-MEL-13 cells were retrovirally transduced to express FLAG-tagged NbSYN87-KEAP1, KEAP1-NbSYN87, KLHDC2-NbSYN87 or KLHL6-NbSYN87. Cells were lysed and 20 μg of lysate protein was resolved by SDS-PAGE and transferred to nitrocellulose membranes for immunoblotting with the indicated antibodies. Quantification of alpha-synuclein normalised to GAPDH control ±SD for N=2 independent repeat experiments.

FIG. 8: Degradation of α-synuclein is comparable to CRISPR KO levels and is reproducible in a neuroblastoma cell line. A) SNCA knockout SK-MEL13 cells were generated using a CRISPR/CAS9 strategy. Wildtype cells were retrovirally infected to express the AdPROM constructs. Cells were lysed alongside KO cells with 20 μg of lysate being resolved by SDS-PAGE. B) Quantification of α-synuclein levels from (A) normalised to loading control+/−SD of n=3 independent experiments. C) Immunofluorescence staining of SK-MEL13 Empty, VHL-NbSYN87 and two SNCA KO clones (#14 and #22) were subjected to anti-α-synuclein immunofluorescence (red) with DNA being stained by DAPI (blue). D) SH-SY5Y cells were retrovirally infected to express either VHL-NbSYN87, VHL, NbSYN87 or with empty vector. Cells were lysed with 20 μg of lysate being resolved by SDS-PAGE, transferred onto nitrocellulose membranes and immunoblotted with the indicated antibodies. E) Quantification of α-synuclein levels from (D) relative to loading control+/−SD of n=3 independent experiments.

FIG. 9: Immunoblotting of samples submitted for total proteomics. SK-MEL13 cells transduced with either empty vector or VHL-NbSYN87 were treated with either DMSO or 40 uM of the proteasomal inhibitor MG132 for 24 hours prior to lysis.

FIG. 10: Targeted degradation of alpha-synuclein through VHL-NbSYN87 is highly specific. Volcano plot of the proteome of VHL-NbSYN87 and control. Fold change of the protein abundances (log 2) are plotted against the t-test p-values (−log 10). The cut off curve indicating significant proteins. Alpha-synuclein is the only protein whose abundance is significantly lower in VHL-NbSYN87 AdPROM transduced cells compared to controls.

FIG. 11: Targeted degradation of alpha-synuclein through VHL-NbSYN87 is specific to the human form of the protein

### DETAILED DESCRIPTION OF EMBODIMENTS OF THE INVENTION AND EXAMPLES

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not limit the scope of the invention.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Ausubel, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); U.S. Pat. No. 4,683,195; Nucleic Acid Hybridization (Harries and Higgins eds. 1984); Transcription and Translation (Hames and Higgins eds. 1984); Culture of Animal Cells (Freshney, Alan R. Liss, Inc., 1987); Immobilized Cells and Enzymes (IRL Press, 1986); Perbal, A Practical Guide to Molecular Cloning (1984); the series, Methods in Enzymology (Abelson and Simon, eds. -in-chief, Academic Press, Inc., New York), specifically, Vols. 154 and 155 (Wu et al. eds.) and Vol. 185, “Gene Expression Technology” (Goeddel, ed.); Gene Transfer Vectors For Mammalian Cells (Miller and Calos eds., 1987, Cold Spring Harbor Laboratory); Immunochemical Methods in Cell and Molecular Biology (Mayer and Walker, eds., Academic Press, London, 1987); Handbook of Experimental Immunology, Vols. I-IV (Weir and Blackwell, eds., 1986); and Manipulating the Mouse Embryo, (Cold Spring Harbor Laboratory Press, Cold Spring Harbor, N.Y., 1986).

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Disclosed herein is proteasomal degradation protein complex comprising an E3 ubiquitin ligase component, such as the CUL2-CRL substrate receptor von-Hippel Lindau tumour suppressor (VHL), tethered to a target specific polypeptide binder. This complex can be termed an affinity-directed protein missile or ‘AdPROM’. The term ‘affinity-directed protein missile (AdPROM)’ or ‘AdPROM system’ as used herein refers to a proteasomal degradation system that comprises an E3 ubiquitin ligase component ‘tethered’ or ‘interlinked’ to a polypeptide binder for target protein recognition.

A target specific polypeptide binder refers to any polypeptide that recognises and binds to a target epitope. Suitably, the target specific polypeptide binder may bind to its target when the target specific polypeptide binder is expressed in a cell or when introduced into a cell. A target specific polypeptide binder may include an antibody, an antibody fragment, a monobody, a nanobody and/or other types of binder based, e.g., on scaffold proteins, can be used. In the present invention any suitable target specific polypeptide binder can be used, and the suitability of any given target specific polypeptide binder can be assessed using the methodologies described herein, e.g. by substituting another target specific polypeptide binder for the nanobody used in the methods set out in the specific examples.

The term ‘antibody’ as used herein is a broad term, and is to be given its ordinary and customary meaning to a person of ordinary skill in the art (and is not to be limited to a special or customized meaning), and refers without limitation to an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also, unless otherwise specified, any antigen binding portion thereof that competes with the intact antibody for specific binding, fusion proteins comprising an antigen binding portion, and any other modified configuration of the immunoglobulin molecule that comprises an antigen recognition site. Antigen binding portions include, for example, Fab, Fab′, F(ab′)2, Fd, Fv, domain antibodies (dAbs, e.g., shark and camelid antibodies), fragments including complementarity determining regions (CDRs), single chain variable fragment antibodies (scFv), maxibodies, minibodies, intrabodies, diabodies, triabodies, tetrabodies, v-NAR and bis-scFv, and polypeptides that contain at least a portion of an immunoglobulin that is sufficient to confer specific antigen binding to the polypeptide. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

The term ‘nanobody’ as used herein may refer to single domain antibody derived from heavy-chain only (VHH) antibodies. VHH antibodies may be of camelid origin including camels, alpacas and llamas. VHH antibodies may be of shark origin. ‘Nanobody’ as used herein includes any of monomeric, dimeric, bispecific or multivalent nanobodies that are specific for a target protein.

The term ‘monobody’ as used herein may refer to synthetic binding proteins constructed using a fibronectin type III domain (FN3) as a molecular scaffold. This class of binding proteins are built upon a diversified library of the 10th FN3 domain of human fibronectin. Various other scaffold protein-based synthetic binding proteins are known in the art, and can be used in the present invention.

The term ‘protein complex’ as used herein refers to two or more associated polypeptide chains. Accordingly, the two or more proteins of the present invention are ‘tethered’ or ‘interlinked’. As such, these terms refer to joining the E3 ligase component to the target specific polypeptide binder. The skilled person would understand these terms to mean directly conjugated or joined via a linker protein.

An E3 ubiquitin ligase component (also called an E3 ubiquitin ligase, E3 ligase or ubiquitin ligase) is a protein that recruits an E2 ubiquitin-conjugating enzyme that has been loaded with ubiquitin and assists or directly catalyses the transfer of ubiquitin from the E2 to the protein substrate. The ubiquitin is attached to a lysine on the target protein by an isopeptide bond. E3 ligases typically interact with both the target protein and the E2 enzyme, and so impart substrate specificity to the E2. However, in the context of the present invention, substrate specificity is conferred by the target specific polypeptide binder. Commonly, E3 ligases polyubiquitinate their substrate with Lys-linked chains of ubiquitin, targeting the substrate for destruction by the proteasome. In the present invention any suitable E3 ligase can be used, and the suitability of any given E3 ligase can be assessed using the methodologies described herein, e.g. by substituting another E3 ligase for VHL in the methods set out in the specific examples.

The term ‘ubiquitin-mediated proteasomal degradation’ or similar terms refers to a key cellular process that controls protein turnover in cells to maintain protein homeostasis. The Cullin-RING ubiquitin E3 ubiquitin ligase (CRL) family, which is comprised of 7 evolutionarily conserved members (CULs 1/2/3/4A/4B/5/7), plays a central role in ubiquitylating and degrading many cellular proteins (20,21). Each CRL machinery consists of a substrate receptor (e.g. von Hippel-Lindau (VHL)), unique adaptors (e.g. Elongin A/B) as well as a RING E3 ligase (Rbx1/2) (21). The substrate receptor subunit recruits and positions the substrate protein proximal to the E3 ligase and its cognate E2-Ub conjugates, which facilitates the ubiquitylation and subsequent degradation of the substrate via the proteasome. For example, under normoxic conditions, the VHL protein recruits the proline-hydroxylated HIF1a transcription factor to CUL2-CRL for its ubiquitylation and degradation. The substrate receptor of the CUL2-CRL machinery can be utilised in the present invention to recruit, ubiquitylate and degrade proteins of interest. Indeed, when VHL tethered to nanobodies or monobodies, and introduced into different cells, there is selective recruitment of the target proteins to CUL2-CRL for an efficient and rapid destruction (18,19). The skilled person will understand that different E3 ligases may result in more or less degradation of the total target protein in a cell when compared to a reference E3 ligase. Suitably, as described herein, VHL is a potent E3 ligase when complexed with NbSYN87, and therefore may be the reference E3 ligase.

As used herein, the term “affinity” refers to the strength of the binding of a single antigen-combining site with an antigenic determinant. Affinity depends on the closeness of stereochemical fit between antibody or antigen binding protein combining sites and antigen determinants, on the size of the area of contact between them, on the distribution of charged and hydrophobic groups, etc. Affinity can be measured by equilibrium analysis or by the Surface Plasmon resonance-“SP” method, for example BIACORE™. The SPR method relies on the phenomenon of surface plasmon resonance (SPR), which occurs when surface plasmon waves are excited at a metal/liquid interface. Light is directed at, and reflected from, the side of the surface not in contact with sample, and SPR causes a reduction in the reflected light intensity at a specific combination of angle and wavelength. Bimolecular binding events cause changes in the refractive index at the surface layer, which are detected as changes in the SPR signal. In the context of the present invention, higher affinity may refer to stronger binding of the polypeptide binder to the target antigen when compared to a competitive binder. Lower affinity may refer to weaker binding of the polypeptide binder to the target antigen when compared to a competitive binder.

“Selectivity” as used herein refers to the binding preference of the polypeptide binder to target epitope. Higher selectivity may refer to a polypeptide binder that exclusively or preferentially binds to the target epitope. In some embodiments, the more selective a polypeptide binder, the less cross-reactive the polypeptide binder is with any protein present. Low selectivity may refer to a polypeptide binder that binds an epitope that is shared with other proteins or is not unique to the target protein.

A “functional variant” of a nucleic acid construct, or amino acid sequence in the context of the present invention is a variant of a reference sequence that retains the ability to function in the same way as the reference sequence. Alternative terms for such functional variants include “biological equivalents” or “equivalents”.

The terms “identity” and “identical” and the like refer to the sequence similarity between two polymeric molecules, e.g., between two nucleic acid molecules, such as between two DNA molecules. Sequence alignments and determination of sequence identity can be done, e.g., using the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Lett 174: 247-250).

Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10.

The National Center for Biotechnology Information (NCBI) Basic Local Alignment Search Tool (BLAST™; Altschul et al. (1990)) is available from several sources, including the National Center for Biotechnology Information (Bethesda, MD), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn) program may be employed using the default parameters. Nucleic acid sequences with even greater similarity to the reference sequences will show increasing percentage identity when assessed by this method. Typically, the percentage sequence identity is calculated over the entire length of the sequence.

For example, a global optimal alignment is suitably found by the Needleman-Wunsch algorithm with the following scoring parameters: Match score: +2, Mismatch score: −3; Gap penalties: gap open 5, gap extension 2. The percentage identity of the resulting optimal global alignment is suitably calculated by the ratio of the number of aligned bases to the total length of the alignment, where the alignment length includes both matches and mismatches, multiplied by 100.

The terms “peptide”, “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “vector” is well known in the art, and as used herein refers to a nucleic acid molecule, e.g. double-stranded DNA, which may have inserted into it a nucleic acid sequence according to the present invention. A vector is suitably used to transport an inserted nucleic acid molecule into a suitable host cell. A vector typically contains all of the necessary elements that permit transcribing the insert nucleic acid molecule, and, preferably, translating the transcript into a polypeptide. A vector typically contains all of the necessary elements such that, once the vector is in a host cell, the vector can replicate independently of, or coincidental with, the host chromosomal DNA; several copies of the vector and its inserted nucleic acid molecule may be generated. Vectors of the present invention can be episomal vectors (i.e., that do not integrate into the genome of a host cell), or can be vectors that integrate into the host cell genome. This definition includes both non-viral and viral vectors. Non-viral vectors include but are not limited to plasmid vectors (e.g. pMA-RQ, pUC vectors, bluescript vectors (pBS) and pBR322 or derivatives thereof that are devoid of bacterial sequences (minicircles)) transposons-based vectors (e.g. PiggyBac (PB) vectors or Sleeping Beauty (SB) vectors), etc. Larger vectors such as artificial chromosomes (bacteria (BAC), yeast (YAC), or human (HAC)) may be used to accommodate larger inserts. Viral vectors are derived from viruses and include but are not limited to retroviral, lentiviral, adeno-associated viral, adenoviral, herpes viral, hepatitis viral vectors or the like. Typically, but not necessarily, viral vectors are replication-deficient as they have lost the ability to propagate in a given cell since viral genes essential for replication have been eliminated from the viral vector. However, some viral vectors can also be adapted to replicate specifically in a given cell, such as e.g. a cancer cell, and are typically used to trigger the (cancer) cell-specific (onco)lysis. Virosomes are a non-limiting example of a vector that comprises both viral and non-viral elements, in particular they combine liposomes with an inactivated HIV or influenza virus (Yamada et al., 2003). Another example encompasses viral vectors mixed with cationic lipids.

The term “CNS cell” or “CNS cells” as used herein includes neurones, astrocytes, oligodendrocytes, microglial cells and/or ependymal cells.

The term ‘pharmaceutically acceptable’ as used herein refers without limitation to an entity or ingredient is one that may be included in the compositions provided herein and that causes no significant adverse toxicological effects in the patient at specified levels, or if levels are not specified, in levels known to be acceptable by those skilled in the art. All ingredients in the compositions described herein are provided at levels that are pharmaceutically acceptable. For clarity, active ingredients may cause one or more side effects and inclusion of the ingredients with a side effect profile that is acceptable from a regulatory perspective for such ingredients will be deemed to be “pharmaceutically acceptable” levels of those ingredients.

The term ‘treatment’ or ‘treating’ as used herein may refer to reducing, ameliorating or eliminating one or more signs, symptoms, or effects of a disease or condition. ‘Treatment’ as used herein includes any treatment of a disease in a mammal, particularly a human, and includes: (a) preventing the disease from occurring in a subject predisposed to the disease or at risk of acquiring the disease but has not yet been diagnosed as having it; (b) inhibiting the disease, i.e., arresting its development; (c) relieving the disease, i.e., causing regression of the disease; and (d) alleviating or reducing any symptoms of the disease.

As used herein, the terms ‘inhibit’, ‘reduce’ and similar terms mean a decrease of at least about 5%, 10%, 15%; 20%, 25%, 35%, 50%, 75%, 80%, 85%, 90%, 95%, 97% or more.

The term ‘subject’ as used herein may be used interchangeably with ‘individual’ or ‘patient’, and refer to any individual subject with a disease or condition in need of prevention or treatment unless otherwise stated. For the purposes of the present disclosure, the subject may be a mammal, preferably a human.

### Advantages:

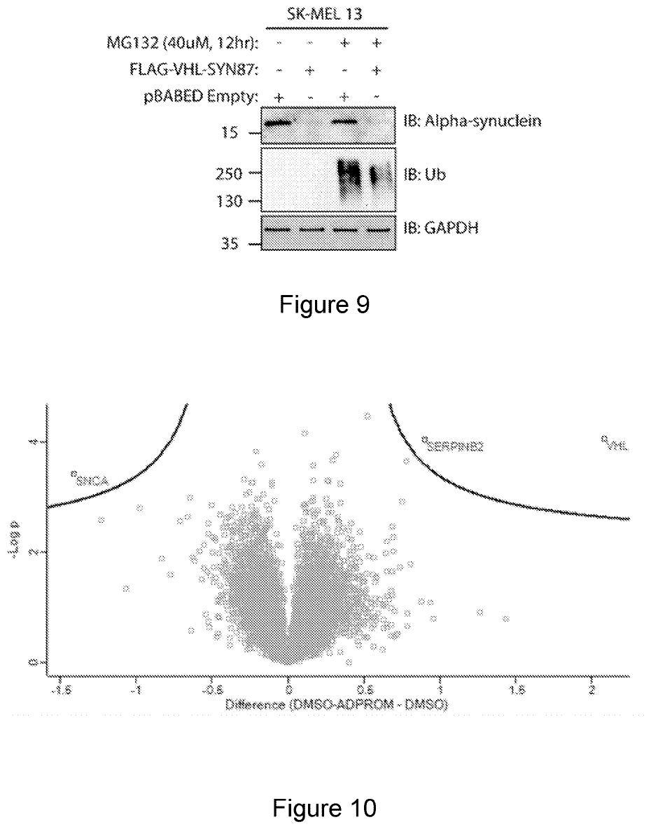

As discussed in detail in the examples below, the inventors have demonstrated the proteasomal degradation protein complex of the present invention is effective and exquisitely selective. By using an unbiased quantitative proteomic approach, it was remarkable that the targeted degradation of α-synuclein protein by AdPROM, α-synuclein was the only target that was degraded out of more than 10,000 proteins identified (FIG. 9). This selectively is advantageous over protein silencing approaches through genomic alteration or transcript inhibition which can result in truncated forms of the protein still being translated or off-target silencing. The proteasomal degradation protein complex being highly selective provides an additional advantage for use a research tool. The proteasomal degradation protein complex can be used to study the effect of removing target proteins independently of genomic or transcriptional modulation.

This system has a further advantage over other targeted proteolysis approaches such as Auxin-inducible degron that requires insertion of the IAA degron sequence into the locus of the protein of interest (POI). The present invention does not require insertion into the POI locus, thus has reduced off-target effects.

Other E3 ligase systems combined with nanobodies such as the ZIF1 proteasomal 30 degradation system have been proposed. However, ZIF1 itself is regulated in different stages of development therefore the POI degradation will compete with endogenous substrates and is therefore less efficient.

The proteasomal degradation protein complex provided herein can be applied in any cell to target POI degradation rapidly. This opens up novel therapeutic options that were not previously available.

Embodiments of the present invention will now be described, by way of non-limiting example, with reference to the accompanying drawings.

### EXAMPLES

### Introduction

Parkinson's disease (PD) is the second most common neurodegenerative disorder, affecting approximately 1 in 500 people in the UK (1). It is characterised by the presence of proteinaceous intracellular inclusions known as Lewy Bodies which are primarily found within the dopaminergic neurons of the substantia nigra pars compacta (2,3). PD is a movement related disorder and the main symptoms include bradykinesia, resting tremor and postural instability as well as reported cognitive symptoms such as depression and dementia (4). The underlying cause of disease development remains unknown however these Lewy bodies are a characteristic hallmark of the disease and could contain potential therapeutic targets. They are reported to consist of a vast number of different proteins but their main component was found to be a small, 140 kDa protein called α-synuclein, encoded for by the SNCA gene (5). Its involvement in the pathogenesis of PD has further been confirmed by the discovery of a number of different mutations of the protein in familial PD, most commonly the A53T mutant (6) as well as A30P, H50Q, E46K, G51D and A53E mutations (7-10). Gene duplications and triplications have also been shown in patients with familial PD (11,12) strengthening the evidence not only for the involvement of α-synuclein in PD but also for the potential of α-synuclein as a therapeutic target. Intracellular inclusions of α-synuclein are also found in a number of different neurodegenerative disorders such as dementia with Lewy bodies and multiple system atrophy (MSA) which have been collectively termed synucleinopathies. Effective treatments against synucleinopathies still remain elusive, and with the increase in life expectancy globally, the need for them has become critical.

Despite intensive research, the function of α-synuclein remains unknown but it is found to be enriched at presynaptic terminals (13) and research suggests that it plays a role in SNARE complex formation allowing for synaptic vesicle release (14). As a member of the intrinsically disordered protein (IDP) family, α-synuclein itself has no tertiary structure and its conformation can be affected by a number of different factors such as the presence of interactors or lipid membranes (15). Its role in PD is further complicated by the fact that the six different mutations of the protein found in patients with familial PD have also been shown to prefer differential oligomeric and fibrillar forms during the aggregation process (16). Interestingly, α-synuclein within these Lewy bodies is nearly all phosphorylated at serine 129 (17) which can be used as a marker to confirm the presence of these aggregated structures as only trace levels of the phosphorylated protein are present in the brains of healthy individuals compared to patients with PD. It is unclear whether this phosphorylation promotes aggregation of the protein or whether it happens after aggregate formation. Whether the toxicity elicited on cells is due to the aggregates themselves, via a toxic gain of function, or due to recruitment of a synuclein from its endogenous site of action, eliciting a loss of function, is also yet to be determined. However, evidence suggests the involvement of aggregated α-synuclein in the pathogenesis and progression of PD and marks it as a potential target for therapeutic intervention. α-synuclein has been deemed an “undruggable” target through conventional small-molecule drug discovery. As such, this study aims to investigate whether a targeted protein degradation approach, through the use of the affinity-directed protein missile (AdPROM) system, developed within the Sapkota lab, could effectively degrade α-synuclein, opening new avenues for therapeutic strategies (18,19).

The Affinity-directed Protein Missile, or AdPROM, system is a novel targeted protein degradation strategy developed to induce the targeted ubiquitination and subsequent proteasomal degradation of both tagged and endogenous. Ubiquitin-mediated proteasomal degradation is a key cellular process that controls protein turnover in cells to maintain protein homeostasis. The Cullin-RING ubiquitin E3 ubiquitin ligase (CRL) family, which is comprised of 7 evolutionarily conserved members (CULs 1/2/3/4A/4B/5/7), plays a central role in ubiquitylating and degrading many cellular proteins (20,21). Each CRL machinery consists of a substrate receptor (e.g. von Hippel-Lindau (VHL)), unique adaptors (e.g. Elongin A/B) as well as a RING E3 ligase (Rbx1/2) (21). The substrate receptor subunit recruits and positions the substrate protein proximal to the E3 ligase and its cognate E2-Ub conjugates, which facilitates the ubiquitylation and subsequent degradation of the substrate via the proteasome. For example, under normoxic conditions, the VHL protein recruits the proline-hydroxylated HIF1α transcription factor to CUL2-CRL for its ubiquitylation and degradation. We can harness the substrate receptor of the CUL2-CRL machinery with our AdPROM system to recruit, ubiquitylate and degrade POIs. Indeed, when we tethered VHL to nanobodies or monobodies, which are small, high affinity polypeptide binders of specific proteins, and introduced them to different cells, we could selectively recruit the target proteins to CUL2-CRL for an efficient and rapid destruction (18,19). So far, we have demonstrated the efficacy of AdPROM in destroying different intracellular endogenous target proteins in different human cell (18,19).

AdPROM consists of two components: an “affinity” probe against a target protein (e.g. a nanobody against α-synuclein) and an interlinked E3 ligase component, such as the CUL2-CRL substrate receptor VHL. For affinity probes, we have used a nanobody against GFP (to degrade GFP-tagged α-synuclein overexpressed in cells, as proof-of-concept) and nanobodies (single chain VHH antibodies generated from Alpacas) that were generated using wild type α-synuclein protein antigen (22,23). One of these nanobodies, NbSYN87, showed particular promise at degrading alpha-synuclein when attached to a PEST proteasome-targeting sequence (24). We show that the use of both an anti-GFP nanobody to degrade GFP-tagged α-synuclein and NbSYN87 to degrade untagged and endogenous alpha-synuclein is achieved through the AdPROM system.

### Materials and Methods

### Plasmids:

All DNA constructs were generated by MRC PPU Reagents and Services and sequenced by MRC PPU DNA Sequencing and Services. All plasmids used in this study are available upon request at https://mrcppureagents.dundee.ac.uk. For SK-MEL13 SNCA KO cells, the following guide RNAs (gRNAs) were generated: sense gRNA (DU64505) and antisense gRNA (DU64516).

### Retroviral Generation of Stable Cell Lines:

Retrovirus was generated using pBABED puro vectors. 6 μg of vector was transiently transfected into a 10 cm dish of approx. 70% confluent HEK 293FT cells alongside 2.2 μg of pCMV5-VSV-G and 3.8 μg of pCMV5-GAG/POL. Briefly, 6 μg of vector, 2.2 μg of VSV-G and 3.8 μg of GAG/POL were added to 300 ul of optimem in one tube. 24ul of 1 mg/ml PEI was added to 300ul of optimem in a second tube. Both tubes were left to incubate for 5 minutes before being mixed and left for a further 20 minutes. Contents of the tube was then added to 9 ml of DMEM media before being placed onto the HEK 293FT cells. Fresh media was added to the cells 16 hours post transfection. 24 hours later, viral media was harvested and passed through a 0.45 μM sterile syringe filter. Target cells at approximately 60% confluency were transduced with the optimised titre of the retroviral medium diluted in fresh medium containing 8 μg/ml polybrene (Sigma-Aldrich) for 24 hr. The retroviral medium was then replaced with selection medium containing 2 μg/ml puromycin for selection of cells which had integrated the constructs. A pool of transduced cells were utilised for subsequent experiments following complete death of non-transduced cells placed under selection in parallel.

### Generation of FIp-In T-Rex Cell Lines:

Flp-In T-Rex U20S and HeLa cells were maintained in complete medium supplemented with 15 μg/ml blasticidin and 100 μg/ml zeocin to maintain expression of the Tet-repressor and integrity of the Flp-recombination site respectively. Cells at 60-70% confluency were transfected with 1 μg of the pcDNA5-FRT/TO vector encoding for the POI as well as 9 μg of the pOG44 Flp recombinase plasmid in 1 ml of Opti-MEM with 20 μl of 1 mg/ml PEI. The transfection mixture was incubated at room temperature for 20 minutes before being added dropwise to the cells. After 24 hours, media was replaced with fresh selection media containing 15 μg/ml blasticidin and 50 μg/ml hygromycin-B. The selection media was replaced every 2-3 days for approximately 2-3 weeks until positive clones were selected. Clones were expanded and verified by immunoblotting. To induce protein expression, cells were incubated with 20 ng/ml doxycycline for 24 hours prior to use, unless stated otherwise in figure legends. In some cases, leaky levels of expression were seen, and these were used to reduce any artefacts potentially causes by overexpression of the POI.

### Cell Lysis:

Cells were first washed twice in ice-cold PBS before being scraped on ice in lysis buffer (50 mM Tris-HCl pH 7.5, 0.27 M sucrose, 150 mM NaCl, 1 mM EGTA, 1 mM EDTA, 1 mM sodium orthovanadate, 10 mM sodium β-glycerophosphate, 50 mM sodium fluoride, 5 mM sodium pyrophosphate and 1% NP-40) supplemented with 1× cOmplete™ protease inhibitor cocktail (Roche). Lysate was transferred to Eppendorf tubes and incubated by rotating for 30 mins to 1 hour at 4° C. Lysates were cleared by centrifugation at 17,000 rpm for 20 minutes at 4° C. Supernatant was transferred to a fresh Eppendorf tube and pellet was discarded. Samples were either processed for use immediately or else snap-frozen in liquid nitrogen and stored at −80° C.

### SDS-PAGE and Western Blotting:

Cell lysates containing equal volumes of protein (10-20 μg) were resolved by SDS-PAGE using Bis-tris gels. Gels were transferred to nitrocellulose membranes and blocked for 1 hour at room temperature with 5% (w/v) non-fat milk(Marvel)/TBS-T. Membranes were incubated overnight at 4° in 5% (w/v) milk/TBS-T containing a dilution of the appropriate primary antibody. Primary antibodies and the dilutions they were used at are anti-α-synuclein (Ab6162, Abcam, 1:500), anti-FLAG HRP (A8592, Sigma, 1:1000), anti-HIF1α (610959, BD Biosciences, 1:1000), anti-GAPDH (2118S, CST, 1:5000) anti-GFP (11814460001, Sigma, 1:1000) anti-Cullin2 (51-1800, Invitrogen, 1:1000), anti-ubiquitin (Z0458, DAKO, 1:1000). Membranes were subsequently washed with TBS-T and incubated for 1 hour at room temperature with HRP-conjugated or fluorescent secondary antibodies. Secondary antibodies used and their dilutions are rabbit anti-sheep-IgG (31480, Thermo Fisher Scientific, 1:2500), goat anti-rabbit-IgG (7074, CST, 1:5000), goat anti-mouse-IgG (31430, Thermo Fisher Scientific, 1:5000), StarBright Blue 700 goat anti-rabbit-IgG (12004161, Bio-Rad, 1:5000). Membranes were again washed in TBS-T and signal detection was performed using ECL (Merck) and the ChemiDoc MP System (Bio-Rad). Image Lab (Bio-Rad) was used to analyse protein bands by densitometry.

### Immunofluorescence Microscopy:

For immunofluorescence imaging, cells were first seeded onto 16 mm diameter circular sterile coverslips in 12-well culture plates and left to adhere overnight. All coverslips were sterilised with 100% (v/v) ethanol prior to use and allowed to dry. Cells were washed twice in PBS before being fixed in 4% (w/v) paraformaldehyde (diluted in PBS) at room temperature for 10 minutes. PFA was removed and the coverslips were washed twice in PBS. Coverslips were then permeabilised with 0.2% (v/v) NP40 in PBS for 3 min. Cells were then blocked by washing twice and a 15-minute incubation in 1% (w/v) BSA/PBS. Coverslips were then incubated for 1.5 hr at room temperature with a 1:100 dilution of anti-α-synuclein antibody (610786, BD Biosciences) in a humidified chamber. They were then washed 3 times (for 10 minutes each) in 0.2% (w/v) BSA/PBS before being incubated with goat anti-mouse-IgG alexa-fluor 594 conjugated secondary antibody (A-11005, Thermo Fisher Scientific) at a dilution of 1:500 for 1 hour at 37° C., protected from light. Coverslips were then washed three times in 0.2% (w/v) BSA/PBS for 10 minutes each with the first wash containing a 1:15,000 dilution of DAPI. They were then briefly immersed in deionised water using a tweezers and placed on a paper towel to air dry. Once dry, approx. 5 μl of Vectashield was dotted onto a glass slide and the coverslip was gently added (cell side down) onto the solution before being sealed with clear nail polish. Cells were imaged on a Deltavision system (Applied Precision) with an immerse-oil 60× or 40× objective and processed with SoftWoRx (Applied Precision). Where applicable, Z-series were obtained and deconvolved using SoftWoRx. Images were processed and figures were made using Adobe Photoshop or OMERO software.

### Proteomic Analysis:

### Cell Lysis, In-Solution Digestion, TMT Labelling and Fractionation

The cells were lysed in the lysis buffer (8M urea, 20 mM HEPES pH 8.0.1 mM sodium orthovanadate 2.5 mM sodium pyrophosphate, 1 mM ß-glycerophosphate), sonicated and centrifuged at 16,000×g for 20 min. Protein concentration was determined using BCA assay (Pierce, Waltham, MA). From each sample 100 μg of protein were reduced and alkylated with 5 mM DTT for 20 min at 60° C. and 10 mM iodoacetamide for 10 min at room temperature respectively. For trypsin digestion, the samples were diluted to reduce the urea concentration <2 M with 20 mM HEPES, pH 8.0 and subjected to digestion with TPCK treated trypsin in 1:20 enzyme to substrate (Worthington Biochemical Corp, Lakewood, NJ) for 12-16 h at room temperature. Digested peptides were acidified by 1% trifluoroacetic acid (TFA) and desalted using C18 Sep-Pak cartridge (Waters, Cat #WAT051910) and dried in vacuum concentrator. These peptides from corresponding samples were labelled with the 16-plex TMT reagents according to the manufacturer instructions. The labelled peptides were pooled together and subjected for the basic pH fractionation. In brief peptide were dissolved buffer A (10 mM ammonium formate, pH 10) and resolved on an XBridge BEH RPLC column (Waters XBridge BEH C18 Column, 130, 5 μm, 4.6 mm×250 mm. #186003010) at a flow rate of 0.3 ml/min by applying gradient of 7-40% (solvent-B, 90% by vol acetonitrile in 10 mM ammonium formate, pH 10) for 80 min into a total of 96 fractions. Each adjacent fraction was pooled together to make 48 fractions for mass spectrometry analysis.

### Mass Spectrometry Analysis

These individual fractions were reconstituted in 0.1% formic acid and analysed on an Orbitrap Fusion Tribrid Mass spectrometer (Thermo Fisher Scientific, San Jose, U.S.A.) interfaced with the Dionex 3000 RSLC nano-liquid chromatography. The peptide samples were enriched on a nano viper trap column (C18, 5 μm, 100 Å, 100 μm×2 cm; PN: 164562, Thermo Scientific) and separated on a 50 cm analytical column (2 μm, 100 Å, 75 μm×50 cm. PN: ES803, Thermo Scientific) by the gradient of solvent B (100% CAN, 0.1% formic acid) for 100 minutes. Mass spectrometer was operated in data-dependent acquisition mode. A survey full scan MS (from m/z 400-1600) was acquired in the Orbitrap with resolution of 120,000 at 200 m/z. Top speed comprising 3 sec cycle time (MS1 and MS2) were used. The precursor ions with charge state ≥2 were isolated in quadrapole with an isolation window of 1.6 m/z and fragmented using HCD fragmentation with 32% normalized collision energy and detected at a mass resolution of 60,000. The AGC target for MS1: 3E5 and 50 ms and for MS2: 5E4. Ion injection times for MS1 50 ms and for MS2 250 ms were used. Dynamic exclusion was set for 30 s with a 10 ppm mass window.

### Data Analysis

The MS/MS searches were carried out using SEQUEST search algorithms against Uniport human protein database using Proteome Discoverer 2.4 (Thermo Fisher Scientific, Bremen, Germany). The workflow included spectrum selector, SEQUEST search nodes, peptide validator, reporter ion quantifier, and percolator node. Oxidation of methionine were set as variable modifications and carbamidomethylation of cysteine and TMT modification at N termini and at lysine was set as a fixed modification. MS and MS/MS mass tolerances were set to 10 ppm and 0.02 Da, respectively. Trypsin was specified as protease and a maximum of one missed cleavage was allowed. Data was also searched against a decoy database and filtered with a 1% false discovery rate (FDR). For the identification of significantly differential proteins, two sample “t-test” was used.

### Example 1—Screening E3 Ligases Capable of Degrading α-Synuclein

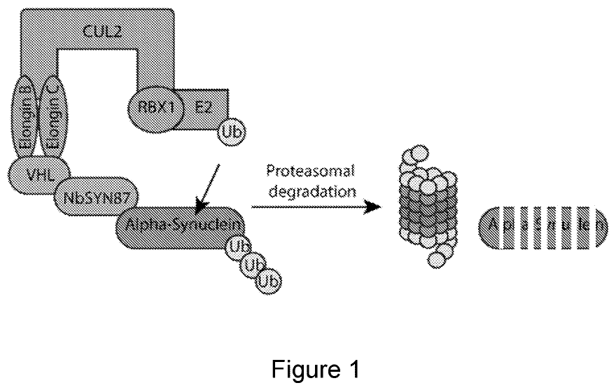

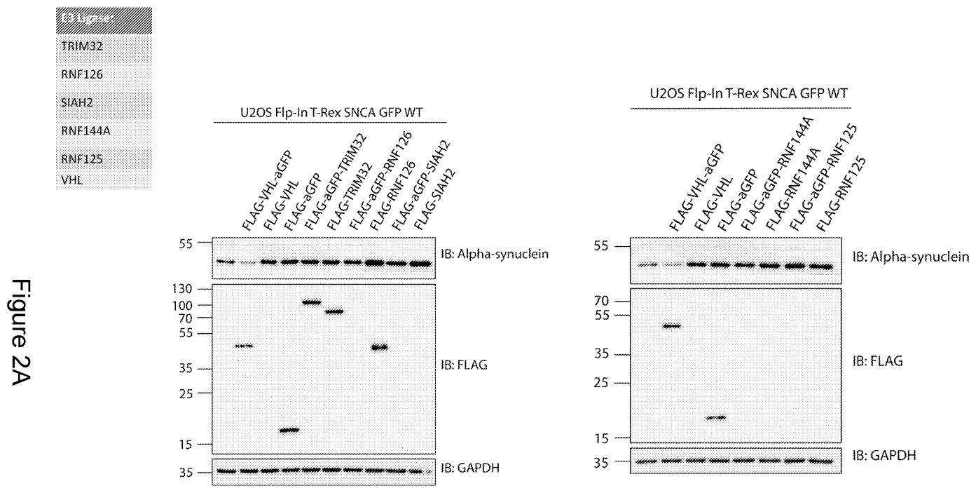

Introduction: The AdPROM system has been designed to link an E3 ligase with a binder of target proteins (see FIG. 1). To determine which E3 ligase is the most efficient at degrading α-synuclein, TRIM32, RNF126, SIAH3, RNF144A, RNF125 and VHL E3 ligases were tethered to a GFP specific nanobody and tested for their capability to degrade GFP-α-synuclein.

Method: Human osteosarcoma epithelial cells (U2OS cell line) were transfected with 1 μg of the pcDNA5-FRT/TO vector encoding for the GFP-α-synuclein as well as 9 μg of the pOG44 Flp recombinase plasmid, alongside the relevant negative control constructs. The positive clones selected over a 2-3 week period. The screen was performed on Flp-IN TRex U2OS cells integrated with GFP-α-synuclein without tetracycline induction. Flp-IN TRex U2OS cells integrated with GFP-α-synuclein expressed a low level of GFP-α-synuclein without inducing expression with tetracycline. GFP-α-synuclein protein degradation was measured in cells expressing TRIM32, RNF126, SIAH3, RNF144A, RNF125 and VHL E3 ligases tethered to a GFP specific nanobody by SDS-PAGE and western blot analysis. Cells were lysed with 20 μg of protein being resolved by SDS-PAGE. Protein was transferred to nitrocellulose membranes and immunoblotted with the indicated antibodies against TRIM32, RNF126, SIAH3, RNF144A, RNF125 and VHL E3 ligases.

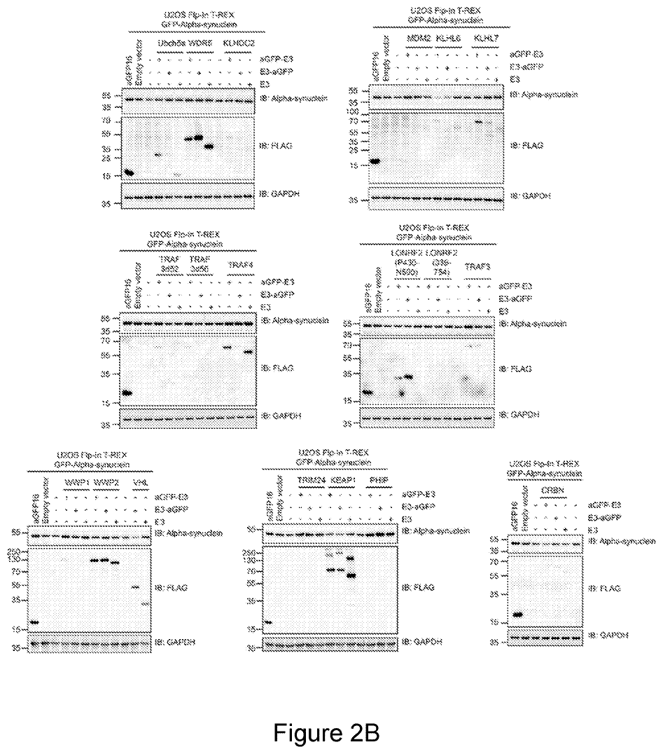

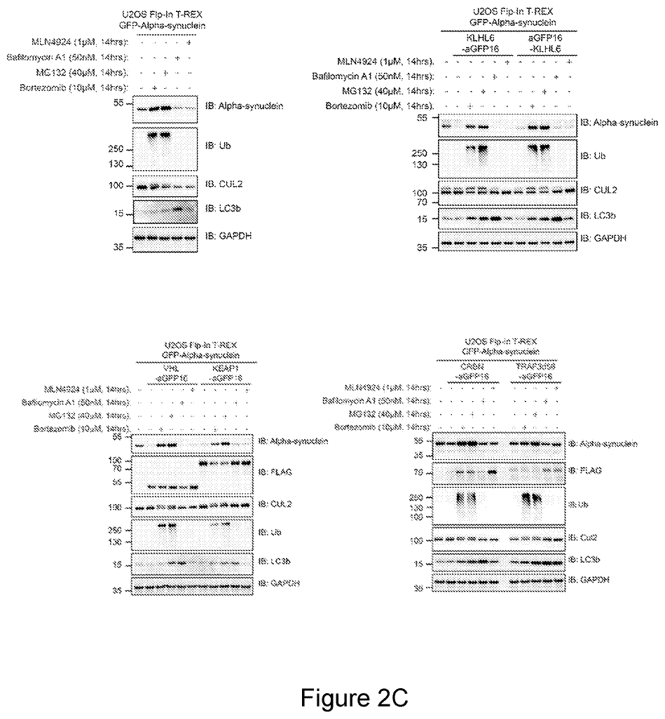

Results: Interestingly, of the E3 ligases tested, only VHL tethered to a GFP specific nanobody resulted in degradation of d α-synuclein. No α-synuclein degradation was observed using TRIM32, RNF126, SIAH3, RNF144A, RNF125 E3 ligases in the AdPROM system (see FIG. 2).

The method as described herein is a suitable tool to test for functional E3 ligases suitable for use in the present invention.

### Example 2—Further Screening of E3 Ligases Capable of Degrading α-Synuclein

Introduction: To identify further E3 ligases suitable for use in degrading α-synuclein when part of the AdPROM conjugate, an expanded E3 ligase AdPROM screen was carried out to test the degradation of GFP-alpha-synuclein.

Method: The method as described in Example 1 was performed with 1 E2 ligase and 18 E3 ligases (Table 1) being expressed with both C- and N-terminal orientation of aGFP16 by retroviral transduction followed by lysis and immunoblotting to identify any degraders.

### Results:

Ubch5a, the only E2 tested in this screen, failed to induce any degradation of alpha-synuclein when used with the AdPROM system compared to non-transduced controls (FIG. 2B). Interestingly, out of the 18 E3 ligase AdPROMs tested, only three appear to induce a decrease in alpha-synuclein levels compared to E3 and aGFP16 alone controls as indicated by immunoblotting (FIG. 2B). VHL-aGFP16, which was shown to degrade alpha-synuclein in the initial screen (Example 1), showed a robust decrease in GFP-alpha-synuclein protein levels (FIG. 2B). KLHL6 and KEAP1 also showed robust decreases in alpha-synuclein levels compared to controls (FIG. 2B). Both orientations of KLHL6 and KEAP1 with aGFP16, C- and N-terminus, showed degradation but the E3-aGFP16 orientation seemed to show slightly better degradation for both E3s. CRBN-aGFP16 also seemed to show a slight degradation of GFP-alpha-synuclein but the reverse orientation showed no changes in protein levels (FIG. 2B). This was similar for TRAF3d56-aGFP16 and aGFP16-KLHDC2 orientations only, both of which showed slight degradation of alpha-synuclein compared to controls, but the reverse orientations did not (FIG. 2B). Interestingly, MDM2 showed no degradation of alpha-synuclein. This was surprising as MDM2, alongside VHL and CRBN, are commonly used E3 ligases for PROTAC development (Bekes, Langley and Crews, 2022).

In order to confirm that the degradation observed with the E3 ligases identified in the screen was occurring through the ubiquitin proteasome pathway, a panel of inhibitors was used: the NEDDylation inhibitor MLN4924, the proteasomal inhibitors MG-132 and bortezomib and the lysosomal inhibitor bafilomycin-A1. Firstly, wildtype U2OS Flp-In T-REX cells were treated for 14 hrs with these inhibitors to determine if treatment alone had any effect on GFP-alpha-synuclein levels. Both bortezomib and MG-132 treatment appeared to slightly increase the levels whilst MLN4924 and bafilomycin-A1 both appeared to slightly decrease levels of the GFP-alpha-synuclein protein compared to DMSO-treated controls (FIG. 2C). Immunoblotting for total Ubiquitin in extracts was carried out to confirm successful proteasomal inhibition, whilst immunoblotting for Cul2 and LC3b were carried out to confirm successful inhibition of NEDDylation and lysosomal degradation respectively (FIG. 2C). As both orientations of KLHL6 with aGFP16 showed robust degradation of GFP-alpha-synuclein in the E3 ligase screen, cells expressing both orientations were treated with the inhibitors. Both MG-132 and bortezomib resulted in rescue of the degradation of alpha-synuclein caused by KLHL6 AdPROMs, confirming this degradation is proteasome-dependent (FIG. 2C). Bafilomycin-A1 and MLN4924 did not result in any rescue KLHL6-AdPROM-dependent degradation of alpha-synuclein, which was unexpected as KLHL6 has been shown to be the substrate receptor for the Cullin 3 RING E3 ligase and as such, MLN4924 treatment should lead to inactivation of Cul3 and rescue of degradation. However, due to the MLN4924 treatment alone causing a decrease in GFP-alpha-synuclein levels, it is thought that any rescue effect is masked, and a longer time point should be used, as previous 24 hr time points showed rescue of any AdPROM-mediated degradation.

Cells expressing VHL-aGFP16 and KEAP1-aGFP16 were also treated with these inhibitors and again, MG-132 and bortezomib treatment rescued degradation of GFP-alpha-synuclein caused by these AdPROMs (FIG. 2C). Again, no rescue was seen with bafilomycin-A1 or MLN4924. This result confirms that the shorter time point of 14 hrs with MLN4924 might not have been insufficient to rescue the degradation of GFP-alpha-synuclein. Two of the E3s which showed slight degradation, CRBN-aGFP16 and TRAF3d56-aGFP16, were also treated with these different inhibitors. The degradation observed with these E3 AdPROMs was not as robust as that seen with VHL, KLHL6 and KEAP1 AdPROMs (FIG. 2C), however the degradation was rescued with bortezomib and MG-132 treatment, confirming this is a proteasome-dependent degradation (FIG. 2C). No rescue was observed when cells were treated with bafilomycin-A1 and MLN4924. Whilst the lack of rescue with MLN4924 is surprising for AdPROMs with CUL substrate receptors, the rescue observed upon proteasomal inhibition confirms that the targeted degradation of GFP-alpha-synuclein occurs through the ubiquitin proteasome system (UPS). The screen further consolidates KLHL6 and KEAP1 as potent proximity-induced degraders of GFP-POIs, including GFP-alpha-synuclein when used with the AdPROM system.

TABLE 1
E2 and E3 Ligases Screened for AdPROM-mediated
degradation of GFP-alpha-synuclein.
Ligases

| | | | |
|---|---|---|---|
| Ubch5a | WDR5 | KLHDC2 | MDM2 |
| KLHL6 | KLHL7 | TRAF3d52 | TRAF3d56 |
| TRAF4 | LONRF2 (P430- | LONRF2 (339-754) | TRAF3 |
| | N500) | | |
| WWP1 | WWP2 | VHL | TRIM24 |
| KEAP1 | PHIP | CRBN | |

### Example 3—VHL-aGFP and VHL-NbSYN87 AdPROM Degrades GFP-Tagged α-Synuclein Overexpressed in U2OS Osteosarcoma Cells

Introduction: To evaluate whether α-synuclein could be targeted for proteolysis by AdPROM, Flp-IN T-Rex U2OS osteosarcoma cells were generated in which a single copy of the N-terminal GFP-tagged α-synuclein, both wildtype and A53T, a common mutant found in familial cases of PD, was integrated into a specific genomic locus containing an upstream Tet-inducible promoter. Using a nanobody directed against GFP tethered to VHL, which has been utilised previously to degrade GFP tagged proteins, the ability to degrade alpha-synuclein through the AdPROM system was determined.

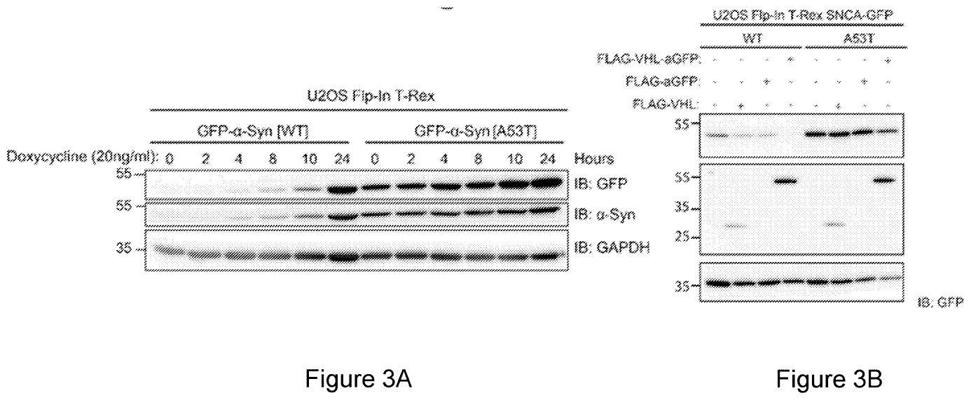

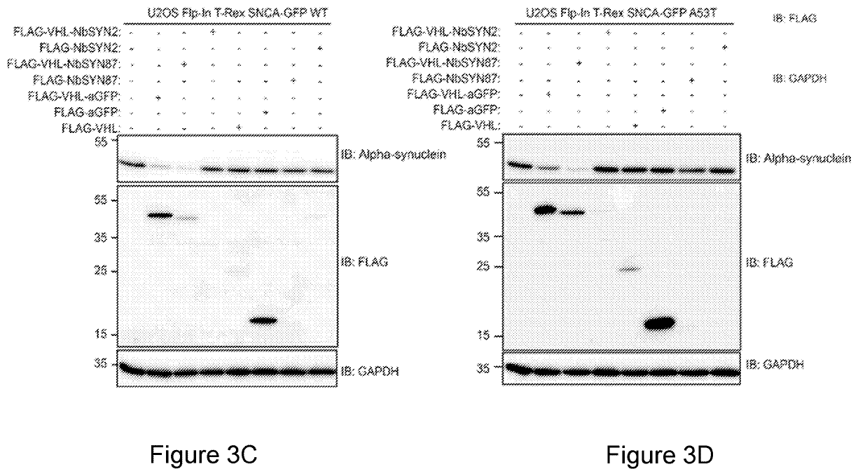

Results: Treatment of Flp-IN T-Rex U2OS osteosarcoma cells with doxycycline over 24 hours resulted in time-dependent increased expression of both GFP-α-synuclein and GFP-α-synuclein-A53T (FIG. 3A). In both cases, some protein expression was detected even in the absence of doxycycline, and substantially more GFP-α-synuclein-A53T mutant was detected relative to GFP-α-synuclein (FIG. 3A). To eliminate the masking of any potential degradation through large overexpression of the protein, the leaky, basal levels of expression to test for targeted degradation of the protein was utilised. Retroviral particles encoding the AdPROM constructs were then generated and used to infect U2OS cells stably expressing GFP-α-synuclein and GFP-α-synuclein-A53T. The levels of GFP-α-synuclein in cells infected with VHL-aGFP AdPROM were substantially lower than in uninfected cells or those infected with VHL alone or aGFP alone controls (FIG. 3B). Similarly, the levels of GFP-α-synuclein-A53T in cells infected with VHL-aGFP AdPROM were much lower than in uninfected cells or those infected with VHL alone or aGFP alone controls (FIG. 3B). These results imply that GFP-tagged α-synuclein or its mutants can be targeted for proteolysis by AdPROM.

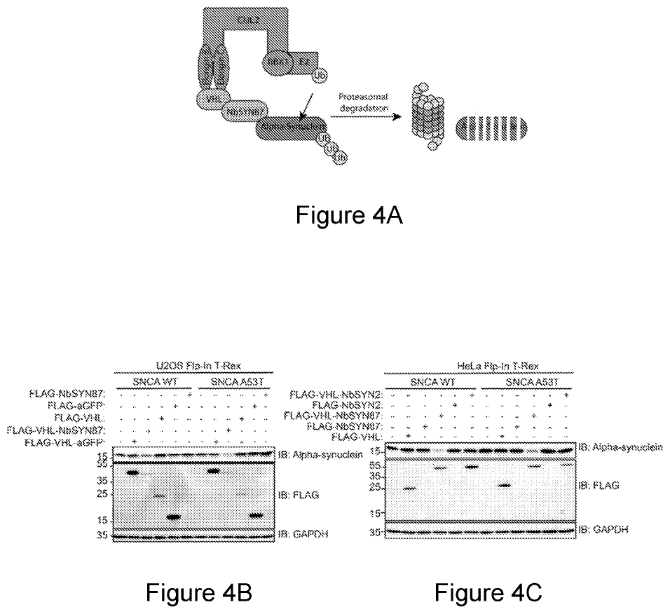

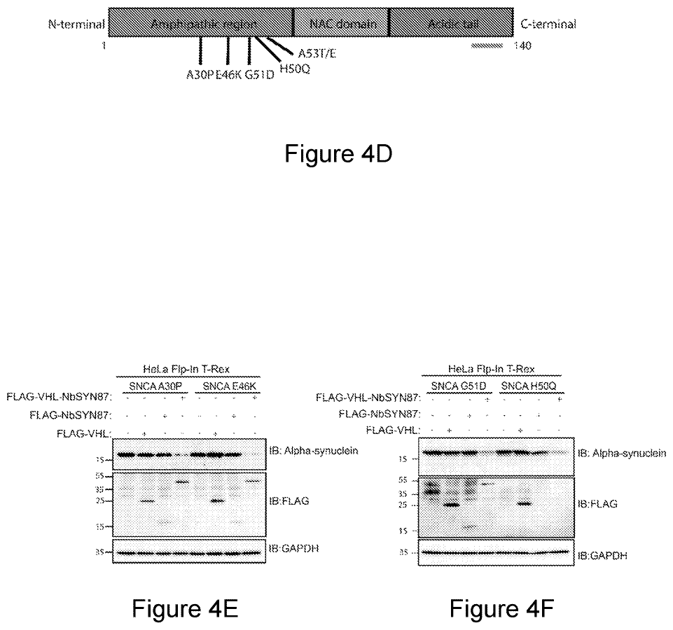

To next determine whether the use of two nanobodies directed against α-synuclein, NbSYN87 and NbSYN2 could be used in place of the GFP nanobody to degrade GFP-tagged α-synuclein. This would facilitate the degradation of untagged and endogenous α-synuclein and remove the need for tagging of the protein. The nanobodies were packaged into VHL-AdPROM constructs (FIG. 4B) and infected U2OS cells stably expressing GFP-α-synuclein or GFP-α-synuclein-A53T. As before, the levels of GFP-α-synuclein in cells infected with VHL-aGFP AdPROM were substantially reduced relative to uninfected cells or cells infected with aGFP and VHL controls (FIG. 4C). Under these conditions, the levels of GFP-α-synuclein were almost undetectable in cells infected with VHL-NbSYN87 AdPROM compared to uninfected cells or those infected with VHL and NbSYN87 controls (FIG. 4C). In contrast, infection of cells with VHL-NbSYN2 or NbSYN2 alone did not affect the levels of GFP-α-synuclein relative to uninfected cells (FIG. 4B). Identical results were obtained in cells stably expressing GFP-α-synuclein-A53T, in that VHL-Nb1 AdPROM resulted in an efficient reduction in GFP-α-synuclein-A53T compared to controls (FIG. 4D). These results suggest that VHL-NbSYN87 AdPROM is effective at targeted proteolysis of GFP-α-synuclein and GFP-α-synuclein-A53T in U2OS cells.

The target specific polypeptide binder of the present invention has to bind in a cell context as exemplified by the ability of VHL-NbSYN87, but not VHL-NbSYN2, to degrade α-synuclein (FIG. 3C). Without wishing to be bound by theory, selectivity of the polypeptide binder is important to the function of the proteasomal degradation protein complex.

To test suitable candidate polypeptide binders the interaction between the polypeptide binder and the target protein a first pass immunoprecipitation (IP) screen was developed to identify candidate binders.

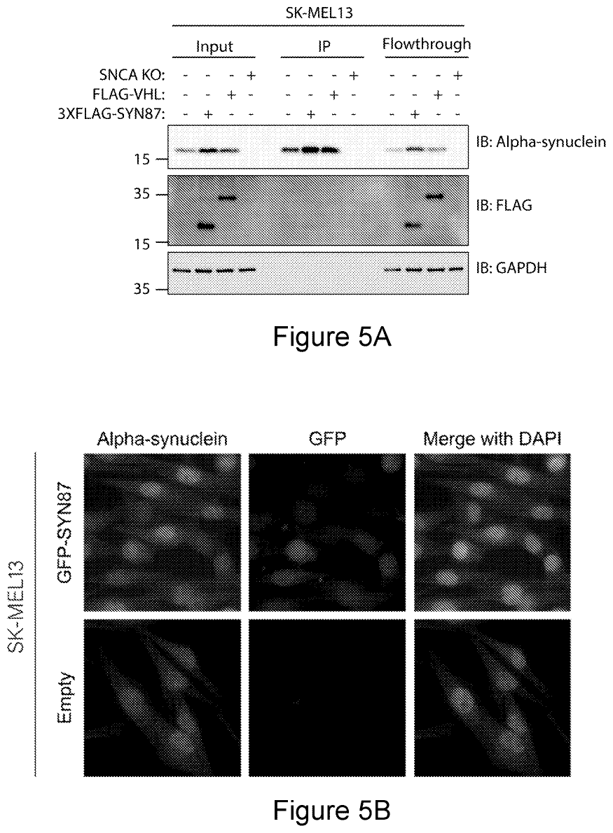

The interaction of NbSyn87 and alpha-synuclein was determined in extracts and cells, with alpha-synuclein knockout cells included as controls for a clean alpha-synuclein IP. To detect the NbSyn87 nanobody an insert 3xFlag tag was used as the one flag tag was undetectable. The NbSYN87 pulled alpha-synuclein down (FIG. 5A). The results were confirmed with immunofluorescence in a cell where GFP-tagged NbSyn87 co-localises completely with endogenous alpha-synuclein (FIG. 5B).

This screen can be utilised to test for suitable selective binders of a target protein of interest. Once a specific binder has been identified it can then be tested with other AdPROM components to determine if the binder and E3 ligase component are active, e.g. in some cases it may be necessary for the binder to interact with the substrate such that it positions the substrate correctly for ubiquitination.

### Example 4—VHL-NbSYN87 AdPROM Degrades Wildtype and Mutant Forms of α-Synuclein Overexpressed in Both U2OS Osteosarcoma Cells and HeLa Cells

Introduction: VHL-NbSYN87 to degrade GFP-α-synuclein, while exciting, could be mediated through ubiquitylation of lysine residues on the GFP tag rather than on the α-synuclein itself. Therefore, in order to test the efficacy of VHL-Nb1 to degrade untagged α-synuclein or α-synuclein-A53T mutant Flp-In T-Rex cells stably integrated with untagged α-synuclein or α-synuclein-A53T mutant under the tetracycline promoter were generated in both U2OS and HeLa cell lines to test the applicability of this targeted degradation in a two different cell lines. Utilising the NbSYN87 nanobody tethered to VHL, we aimed to test the degradability of untagged α-synuclein through the AdPROM system (FIG. 4A).

Results: U2OS cells were infected with VHL-aGFP AdPROM, no reduction in the levels of untagged α-synuclein or α-synuclein-A53T were observed relative to uninfected cells, or those infected with VHL or aGFP controls. Cells infected with VHL-NbSYN87 AdPROM showed a substantial decrease in levels of both α-synuclein and α-synuclein-A53T, relative to uninfected cells or those infected with VHL-aGFP, VHL, aGFP or Nb1 controls (FIG. 4B). This degradation of α-synuclein in cells expressing VHL-NbSYN87 was also reproducible in the HeLa Flp-In T-Rex cells expressing the untagged form of the protein (FIG. 4B). These cells were also retrovirally infected to express VHL-NbSYN2 and as previously shown with the GFP-tagged α-synuclein cell lines, no degradation was observed with the use of this nanobody (FIG. 4B). These results suggest that the VHL-NbSYN87 is capable of degrading untagged α-synuclein and α-synuclein-A53T overexpressed in both cell lines tested, HeLa and U2OS. α-synuclein is a 140 kDa protein that consists of three main domains, an N-terminal amphipathic α-helical region, a central hydrophobic core known as the non-amyloid component or NAC domain and an acidic C-terminal tail. There are six mutations of α-synuclein which have been found in familial cases of PD and they are all contained within the N-terminal region of the protein. The epitope for the NbSYN87 nanobody is in the C-terminal region of the protein, indicated by the red line (FIG. 4D). This implies that this approach could be used to degrade all mutant forms of the protein currently described in clinical cases of familial Parkinson's disease.

HeLa Flp-In T-Rex cells were generated to express the other four mutants of the protein, A30P, E46K, G51D and H50Q. When retrovirally infected to express either VHL-NbSYN87, VHL or NbSYN87 alone, significant degradation of all four mutants of α-synuclein was observed in cells expressing the VHL-NbSYN87 AdPROM (FIG. 4E-F). This indicates that the VHL-NbSYN87 is capable of degrading untagged α-synuclein and the clinically relevant mutated forms of the protein.

### Example 5—VHL-NbSYN87 AdPROM Degrades Endogenous α-Synuclein

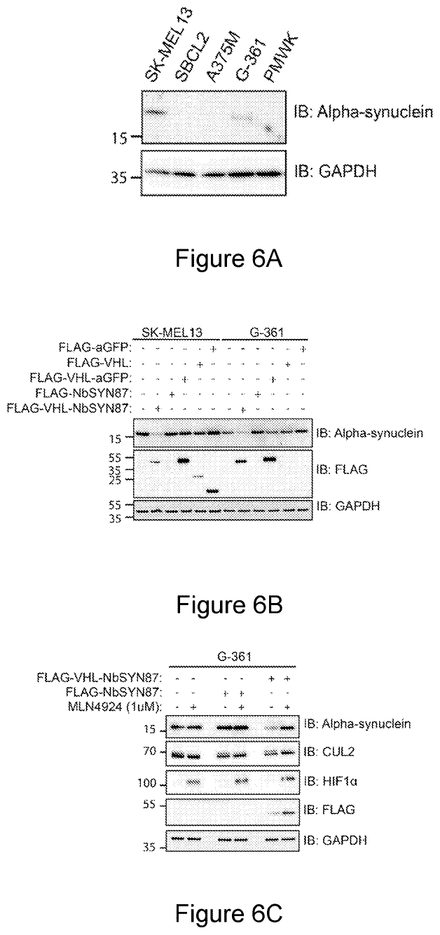

To explore whether VHL-NbSYN87 AdPROM could target the degradation of physiological levels of α-synuclein. In the absence of any appropriate neuronal cells at hand, commonly used human melanoma cell lines were probed to uncover any that expressed detectable levels of α-synuclein, as previous studies report endogenous levels of the protein in certain melanoma cell lines (25). Two melanoma cell lines, namely SK-MEL13 and G-361, did indeed express α-synuclein protein levels that could be detected by Western blotting, while most other cell lines we tested did not (FIG. 6A).

When both SK-MEL13 and G361 cells were infected with VHL-NbSYN87 AdPROM, there was an almost complete disappearance in levels of endogenous α-synuclein relative to uninfected cells or those infected with VHL-aGFP, VHL, aGFP or NbSYN87 controls (FIG. 6B). These results suggest that the VHL-NbSYN87 can degrade physiological α-synuclein protein from cells that express α-synuclein and has potential implications in therapeutic removal of α-synuclein. In order to determine whether the loss in levels of endogenous α-synuclein by VHL-NbSYN87 was indeed mediated by the CUL2-CRL machinery, we treated G-361 cells with the pan-Cullin Neddylation inhibitor MLN4924 for 24 hours prior to cell lysis. As expected, treatment of G-361 cells with MLN4924 resulted in robust inhibition of CUL2 Neddylation and a concurrent stabilization of its endogenous target, HIF1α (FIG. 6C). Under these conditions, the loss in levels of endogenous α-synuclein was partially rescued by treatment of cells with MLN4924 after 24 hrs (FIG. 6C). Importantly, the expression of VHL-NbSYN87 had no effect on the levels of endogenous HIF1α relative to controls, suggesting the AdPROM system does not interfere with the endogenous CUL2 E3 ubiquitin ligase machinery (FIG. 6C).

### Example 6—KLHL6-NbSYN87, KEAP1-NbSYN87 and KLHDC2-NbSYN87 AdPROMs Degrade Endogenous α-Synuclein

Introduction: As discussed above, KLHL6, KLHDC2 and KEAP1 in addition to VHL were found to be capable of degrading GFP-alpha-synuclein in the E3 ligase screen. The ability of KLHL6, KLHDC2 and KEAP1 to degrade the endogenous α-synuclein was then determined.

Method: KLHL6, KLHDC2 and KEAP1 were each cloned into a vector with NbSYN87. Both orientations of KEAP1 were tested to determine if there were any differences between having NbSYN87 on the N- or C-terminus whereas for KLHL6 and KLHDC2 only the C-terminal nanobody orientation was examined. SK-MEL-13 cells were retrovirally transduced to express these constructs and, after puromycin selection, were lysed and immunoblotting was carried out to examine any effects on alpha-synuclein levels.

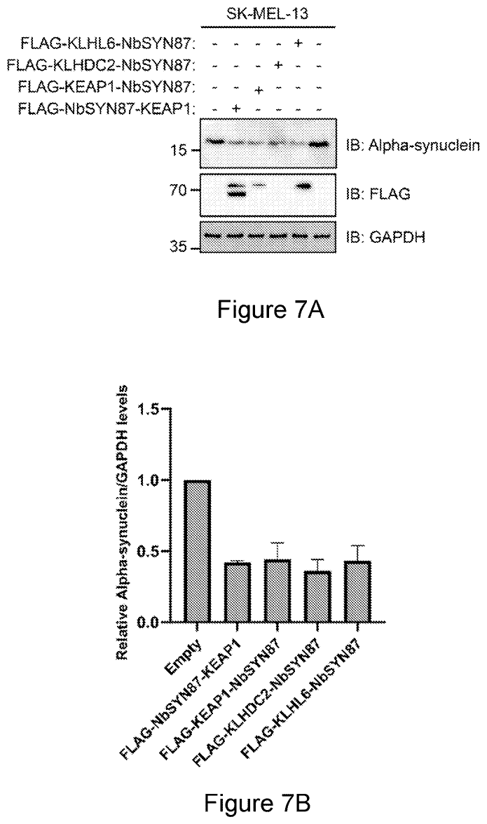

Results: All AdPROMs, NbSYN87-KEAP1, KEAP1-NbSYN87 KLHL6-NbSYN87 and KLHDC2-NbSYN87 induced degradation of the endogenous alpha-synuclein protein in SK-MEL-13 cells relative to empty vector controls (FIG. 7A). Quantification of two biological repeats showed that the level of alpha-synuclein degradation was about 50% compared to empty vector control (FIG. 7B). The E3 screen has unveiled three additional E3 ligases capable of degrading endogenous alpha-synuclein through proximity-induction that could provide alternatives to the near complete degradation achieved with VHL-NbSYN87.

### Example 7—Targeted Degradation of α-Synuclein Through VHL-NbSYN87 is Comparable to Knockout Cells and is Reproducible in a Neuroblastoma Cell Line

Due to the extent of the reduction in α-synuclein levels in these melanoma cells expressing VHL-NbSYN87 we sought to quantify this degradation and compare it to complete KO cells. Previous studies carried out in dopaminergic neurons differentiated from iPSCs from a SNCA triplication patient showed that complete KO of alpha-synuclein reduced seeded aggregation compared to wildtype cells (26). Other studies using KO cells have shown that abolishment of α-synuclein protein levels has resulted in resistance to certain neurotoxic models of parkinsonism (27-29). Due to the difficulty in utilising a CRISPR/Cas9 strategy for therapeutic development, we wanted to examine the levels of degradation of alpha-synuclein compared to total KO cells to determine whether the AdPROM system could be utilised as a similar strategy to target alpha-synuclein.

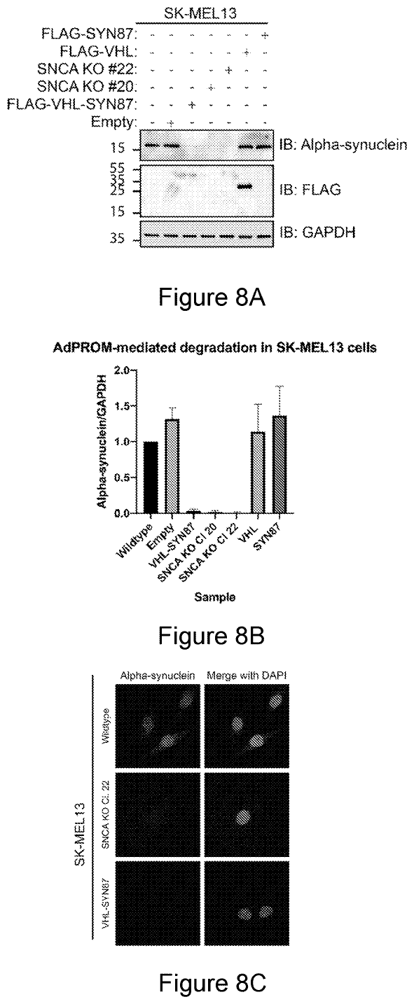

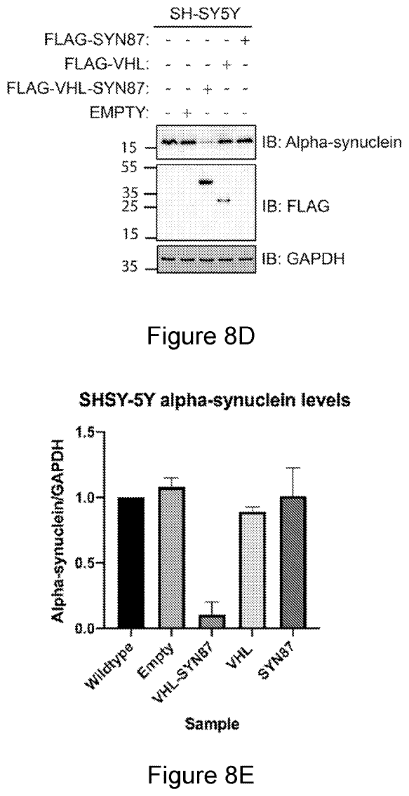

Knock out cells were generated using a CRISPR/Cas9 strategy in SK-MEL13 cells. Cells retrovirally infected to express either VHL-NbSYN87, VHL or NbSYN87 were lysed alongside two clones of SK-MEL13 SNCA KO cells obtained (#20 and #22). The reduction in α-synuclein levels obtained by targeted degradation through the AdPROM system was comparable to total KO cells (FIG. 8A) and this was confirmed by three independent experiments and subsequent quantification of relative α-synuclein levels (FIG. 8B). Immunostaining of SK-MEL13 cells for α-synuclein shows a lack of any significant staining in KO cells and VHL-NbSYN87 cells compared to the wildtype cells (FIG. 8C). This highlights the degradative capacity of the AdPROM system and due to the previous studies carried out using KO cells suggests a potential therapeutic value in this targeted degradation of alpha-synuclein by the AdPROM system.

Due to the lack of availability of primary neuronal cells, we next sought to show the applicability of this approach in a neuroblastoma cell line, SH-SY5Y cells. These cells, when retrovirally infected to express either VHL-NbSYN87 or the appropriate controls, showed significant reduction of the endogenous α-synuclein levels only in cells expressing the VHL-NbSYN87 construct (FIG. 8D). Quantification of relative α-synuclein levels from three independent repeats of this experiment show a significant decrease in protein levels only in VHL-NbSYN87 expressing cells (FIG. 8E). This confirms the feasibility of this approach in a more neuronal like cell line suggesting the possibility of this approach in primary neurons.

### Example 8—Targeted Degradation of α-Synuclein Through VHL-NbSYN87 is Highly Specific

Introduction: To determine the specificity of this targeted degradation of α-synuclein we looked at the global quantitative proteomic changes upon expression of the VHL-NbSYN87 AdPROM in SK-MEL13 cells.

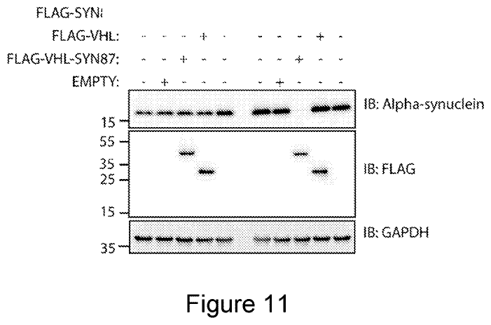

SK-MEL13 cells transduced with viruses encoding either the pBABED empty vector control or the VHL-NbSYN87 AdPROM were compared. In these cells, a decrease in alpha-synuclein levels was confirmed by immunoblotting prior to processing the samples for proteomic analysis (FIG. 9). Samples treated with the proteasomal inhibitor MG132 were also prepared for total proteomics to try and identify potential ubiquitin sites which is currently ongoing (FIG. 10). Excitingly, α-synuclein was the only protein whose levels decreased significantly in cells expressing the VHL-NbSYN87 construct compared to the empty vector control cells (FIG. 10). VHL protein levels increased significantly which was expected due to the transduction of the cells with the VHL-NbSYN87 plasmid. Another protein, plasminogen activator inhibitor 2 (PAI2), encoded for by the SerpinB2 gene was also increased in the VHL-NbSYN87 cells compared to the cells treated with empty vector (FIG. 10). Protein levels of the other two members of the synuclein family, β- and γ-synuclein, were unchanged across samples indicating the high specificity of this approach. The nanobody itself has also been shown to be highly specific for human α-synuclein. U2OS FIp-In T-Rex cells expressing mouse alpha-synuclein were retrovirally transduced to express VHL-NbSYN87 or VHL and NbSYN87 alone and no degradation of the mouse α-synuclein was seen in cells expressing VHL-NbSYN87, even though there is approximately 97% sequence identity between human and mouse forms of the protein (FIG. 11).

Sequences (5′ to 3′)

DNA sequence encoding NbSYN87 (SEQ ID NO: 1):
caggtgcagctgcaggaaagcggcggcggcagcgtgcagaccggcggcagcctgcgcctgagctgcgtggcgagcggcta
tagcggctatatggcgtggtttcgccaggcgccgggcaaagaacgcgaaggcattgcggcgatttatcgcggcgataaaattac
ctattatgcgcatagcgtgcagggccgctttaccattagccaggcgaacgcgaaaaacaccgtgtatctgctgatgaacagcctg
aaaccggaagataccgcgatttattattgcgcggcgcgccgcgtggtggcggatagcccgctgctgagcaaaacctatgcgtat
tggggccagggcacccaggtgaccgtgagcagctga
NbSYN87 amino acid sequence (SEQ ID NO: 12):
QVQLQESGGGSVQTGGSLRLSCVASGYSGYMAWFRQAPGKEREGIAAIYRGDKITYYAHS
VQGRFTISQANAKNTVYLLMNSLKPEDTAIYYCAARRVVADSPLLSKTYAYWGQGTQVTVS
S*
DNA sequence encoding VHL (SEQ ID NO: 2):
atgccccggagggcggagaactgggacgaggccgaggtaggcgcggaggaggcaggcgtcgaagagtacggccctgaa
gaagacggcggggaggagtcgggcgccgaggagtccggcccggaagagtccggcccggaggaactgggcgccgagga
ggagatggaggccggtcggccgcggcccgtgctgcgctcggtgaactcgcgcgagccctcccaggtcatcttctgcaatcgca
gtccgcgcgtcgtgctgcccgtatggctcaacttcgacggcgagccgcagccctacccaacgctgccgcctggcacgggccgc
cgcatccacagctaccgaggtcacctttggctcttcagagatgcagggacacacgatgggcttctggttaaccaaactgaattattt
gtgccatctctcaatgttgacggacagcctatttttgccaatatcacactgccagtgtatactctgaaagagcgatgcctccaggttgt
ccggagcctagtcaagcctgagaattacaggagactggacatcgtcaggtcgctctacgaagatctggaagaccacccaaat
gtgcagaaagacctggagcggctgacacaggagcgcattgcacatcaacggatgggagat
VHL amino acid sequence (SEQ ID NO: 13):
MPRRAENWDEAEVGAEEAGVEEYGPEEDGGEESGAEESGPEESGPEELGAEEEMEAGR
PRPVLRSVNSREPSQVIFCNRSPRVVLPVWLNFDGEPQPYPTLPPGTGRRIHSYRGHLWLF
RDAGTHDGLLVNQTELFVPSLNVDGQPIFANITLPVYTLKERCLQVVRSLVKPENYRRLDIV
RSLYEDLEDHPNVQKDLERLTQERIAHQRMGD
DNA sequence encoding VHL-NbSYN87 (SEQ ID NO: 3):
atgccccggagggcggagaactgggacgaggccgaggtaggcgcggaggaggcaggcgtcgaagagtacggccctgaa
gaagacggcggggaggagtcgggcgccgaggagtccggcccggaagagtccggcccggaggaactgggcgccgagga
ggagatggaggccggtcggccgcggcccgtgctgcgctcggtgaactcgcgcgagccctcccaggtcatcttctgcaatcgca
gtccgcgcgtcgtgctgcccgtatggctcaacttcgacggcgagccgcagccctacccaacgctgccgcctggcacgggccgc
cgcatccacagctaccgaggtcacctttggctcttcagagatgcagggacacacgatgggcttctggttaaccaaactgaattattt
gtgccatctctcaatgttgacggacagcctatttttgccaatatcacactgccagtgtatactctgaaagagcgatgcctccaggttgt
ccggagcctagtcaagcctgagaattacaggagactggacatcgtcaggtcgctctacgaagatctggaagaccacccaaat
gtgcagaaagacctggagcggctgacacaggagcgcattgcacatcaacggatgggagatggtggaggcggaggtgaattc
gccatgcaggtgcagctgcaggaaagcggcggcggcagcgtgcagaccggcggcagcctgcgcctgagctgcgtggcgag
cggctatagcggctatatggcgtggtttcgccaggcgccgggcaaagaacgcgaaggcattgcggcgatttatcgcggcgata
aaattacctattatgcgcatagcgtgcagggccgctttaccattagccaggcgaacgcgaaaaacaccgtgtatctgctgatgaa
cagcctgaaaccggaagataccgcgatttattattgcgcggcgcgccgcgtggtggcggatagcccgctgctgagcaaaacct
atgcgtattggggccagggcacccaggtgaccgtgagcagc
VHL-NbSYN87 amino acid sequence (SEQ ID NO: 14)
MPRRAENWDEAEVGAEEAGVEEYGPEEDGGEESGAEESGPEESGPEELGAEEEMEAGR
PRPVLRSVNSREPSQVIFCNRSPRVVLPVWLNFDGEPQPYPTLPPGTGRRIHSYRGHLWLF
RDAGTHDGLLVNQTELFVPSLNVDGQPIFANITLPVYTLKERCLQVVRSLVKPENYRRLDIV
RSLYEDLEDHPNVQKDLERLTQERIAHQRMGDGGGGGEFAMQVQLQESGGGSVQTGGSL
RLSCVASGYSGYMAWFRQAPGKEREGIAAIYRGDKITYYAHSVQGRFTISQANAKNTVYLL
MNSLKPEDTAIYYCAARRVVADSPLLSKTYAYWGQGTQVTVSS
GILEDMPVDPDNEAYEMPSEEGYQDYEPEA (SEQ ID NO: 4)
VDPDNEAYEMPS (SEQ ID NO: 5)
DNA sequence encoding KLHL6 (SEQ ID NO: 6):
ATGTTGATGGCAGGACAAAGGGGCGCCTGGACCATGGGTGATGTGGTCGAGAAGAGT
TTGGAAGGGCCCCTGGCACCTTCTACAGATGAGCCCTCCCAGAAAACAGGAGACTTGG
TCGAGATCTTAAATGGGGAAAAGGTCAAATTTGACGACGCGGGACTCTCCTTAATTCTT
CAGAATGGCCTGGAAACCCTGCGAATGGAAAACGCTCTGACAGATGTCATCTTGTGTG
TGGACATTCAGGAATTCTCCTGCCACCGCGTGGTGCTTGCCGCAGCCAGCAACTATTT
CAGGGCCATGTTCTGCAACGATTTAAAGGAAAAGTATGAGAAAAGGATCATTATTAAAG
GGGTTGATGCTGAGACCATGCACACTCTGTTGGACTACACGTACACCAGCAAGGCGCT
GATCACCAAGCAGAATGTCCAGCGGGTCCTGGAGGCTGCTAACCTCTTCCAGTTCCTG
CGGATGGTGGATGCCTGTGCCAGCTTCCTCACTGAAGCCTTGAACCCAGAAAACTGCG
TTGGAATACTGAGGCTGGCTGACACACACTCGCTGGACAGTCTAAAGAAGCAGGTTCA
GAGTTACATCATTCAAAACTTTGTGCAGATTCTGAACTCTGAGGAGTTTCTTGACCTGCC
CGTGGACACTCTGCACCACATCTTGAAGAGTGATGACCTTTACGTGACCGAGGAGGCT
CAGGTGTTTGAGACCGTGATGAGCTGGGTCCGGCACAAGCCATCAGAACGACTCTGCT
TACTCCCCTATGTCCTCGAGAACGTGCGCTTACCGCTTCTGGACCCGTGGTACTTTGTG
GAGACGGTGGAAGCAGATCCTCTCATCAGGCAGTGCCCAGAGGTCTTCCCGCTGCTCC
AGGAAGCCAGGATGTACCACCTTTCTGGCAATGAGATCATTTCGGAACGCACCAAGCC
CAGGATGCATGAGTTCCAGTCTGAGGTGTTCATGATCATTGGCGGCTGCACGAAGGAT
GAACGGTTTGTGGCAGAGGTGACCTGCCTGGACCCCCTGAGGCGCAGCCGCCTGGAG
GTGGCCAAGCTCCCGCTAACAGAGCATGAGCTGGAGAGTGAGAATAAGAAGTGGGTG
GAGTTTGCATGCGTGACATTGAAAAATGAGGTCTACATCTCAGGTGGCAAAGAAACACA
GCATGATGTTTGGAAATATAATTCTTCGATCAACAAGTGGATTCAGATTGAGTATTTAAA
CATAGGCCGCTGGAGGCATAAGATGGTGGTGTTGGGTGGCAAGGTCTATGTGATCGGA
GGCTTTGACGGCTTACAGAGAATCAACAACGTGGAGACCTACGACCCCTTTCACAACT
GCTGGTCAGAGGCCGCACCCCTCCTTGTCCATGTCAGTTCCTTTGCAGCCACCAGCCA
TAAGAAGAAGCTGTATGTGATCGGGGGAGGGCCCAATGGGAAACTGGCCACAGACAA
GACTCAGTGTTATGACCCTTCCACCAACAAGTGGAGTTTGAAGGCGGCCATGCCCGTG
GAGGCTAAATGCATCAATGCAGTGAGTTTCCGGGACCGCATCTATGTCGTTGGTGGGG
CCATGAGAGCGCTGTACGCCTACAGCCCGCTGGAAGACAGCTGGTGCCTGGTGACCC
AGCTCAGCCACGAGCGGGCCAGCTGCGGTATCGCGCCCTGCAACAACCGGCTCTACA
TCACCGGCGGGGGGACGAGAAGAACGAGGTTATCGCCACGGTGCTGTGCTGGGACC
CCGAGGCCCAGAAACTGACAGAGGAGTGCGTCCTGCCCCGGGGCGTGTCGCACCACG
GCAGCGTCACCATCAGGAAGTCGTACACCCACATCCGCAGGATCGTGCCCGGAGCAG
TGTCTGTCTGA
KLHL6 amino acid sequence (SEQ ID NO: 15):
MLMAGQRGAWTMGDVVEKSLEGPLAPSTDEPSQKTGDLVEILNGEKVKFDDAGLSLILQN
GLETLRMENALTDVILCVDIQEFSCHRVVLAAASNYFRAMFCNDLKEKYEKRIIIKGVDAETM
HTLLDYTYTSKALITKQNVQRVLEAANLFQFLRMVDACASFLTEALNPENCVGILRLADTHSL
DSLKKQVQSYIIQNFVQILNSEEFLDLPVDTLHHILKSDDLYVTEEAQVFETVMSWVRHKPSE
RLCLLPYVLENVRLPLLDPWYFVETVEADPLIRQCPEVFPLLQEARMYHLSGNEIISERTKPR
MHEFQSEVFMIIGGCTKDERFVAEVTCLDPLRRSRLEVAKLPLTEHELESENKKWVEFACV
TLKNEVYISGGKETQHDVWKYNSSINKWIQIEYLNIGRWRHKMVVLGGKVYVIGGFDGLQRI
NNVETYDPFHNCWSEAAPLLVHVSSFAATSHKKKLYVIGGGPNGKLATDKTQCYDPSTNK
WSLKAAMPVEAKCINAVSFRDRIYVVGGAMRALYAYSPLEDSWCLVTQLSHERASCGIAPC
NNRLYITGGRDEKNEVIATVLCWDPEAQKLTEECVLPRGVSHHGSVTIRKSYTHIRRIVPGA
VSV*
DNA sequence encoding KEAP1 (SEQ ID NO: 7):
atgcagccagatcccaggcctagcggggctggggcctgctgccgattcctgcccctgcagtcacagtgccctgagggggcagg
ggacgcggtgatgtacgcctccactgagtgcaaggcggaggtgacgccctcccagcatggcaaccgcaccttcagctacacc
ctggaggatcataccaagcaggcctttggcatcatgaacgagctgcggctcagccagcagctgtgtgacgtcacactgcaggtc
aagtaccaggatgcaccggccgcccagttcatggcccacaaggtggtgctggcctcatccagccctgtcttcaaggccatgttca
ccaacgggctgcgggagcagggcatggaggtggtgtccattgagggtatccaccccaaggtcatggagcgcctcattgaattc
gcctacacggcctccatctccatgggcgagaagtgtgtcctccacgtcatgaacggtgctgtcatgtaccagatcgacagcgttgt
ccgtgcctgcagtgacttcctggtgcagcagctggaccccagcaatgccatcggcatcgccaacttcgctgagcagattggctgt
gtggagttgcaccagcgtgcccgggagtacatctacatgcattttggggaggtggccaagcaagaggagttcttcaacctgtccc
actgccaactggtgaccctcatcagccgggacgacctgaacgtgcgctgcgagtccgaggtcttccacgcctgcatcaactggg
tcaagtacgactgcgaacagcgacggttctacgtccaggcgctgctgcgggccgtgcgctgccactcgttgacgccgaacttcct
gcagatgcagctgcagaagtgcgagatcctgcagtccgactcccgctgcaaggactacctggtcaagatcttcgaggagctca
ccctgcacaagcccacgcaggtgatgccctgccgggcgcccaaggtgggccgcctgatctacaccgcgggcggctacttccg
acagtcgctcagctacctggaggcttacaaccccagtgacggcacctggctccggttggcggacctgcaggtgccgcggagcg
gcctggccggctgcgtggtgggcgggctgttgtacgccgtgggcggcaggaacaactcgcccgacggcaacaccgactccag
cgccctggactgttacaaccccatgaccaatcagtggtcgccctgcgcccccatgagcgtgccccgtaaccgcatcggggggg
ggtcatcgatggccacatctatgccgtcggcggctcccacggctgcatccaccacaacagtgtggagaggtatgagccagagc
gggatgagtggcacttggtggccccaatgctgacacgaaggatcggggtgggcgtggctgtcctcaatcgtctcctttatgccgtg
gggggctttgacgggacaaaccgccttaattcagctgagtgttactacccagagaggaacgagtggcgaatgatcacagcaat
gaacaccatccgaagcggggcaggcgtctgcgtcctgcacaactgtatctatgctgctgggggctatgatggtcaggaccagct
gaacagcgtggagcgctacgatgtggaaacagagacgtggactttcgtagcccccatgaagcaccggcgaagtgccctggg
gatcactgtccaccaggggagaatctacgtccttggaggctatgatggtcacacgttcctggacagtgtggagtgttacgaccca
gatacagacacctggagcgaggtgacccgaatgacatcgggccggagtggggtgggcgtggctgtcaccatggagccctgc
cggaagcagattgaccagcagaactgtacctgttga
KEAP1 amino acid sequence (SEQ ID NO: 16):
MQPDPRPSGAGACCRFLPLQSQCPEGAGDAVMYASTECKAEVTPSQHGNRTFSYTLEDH
TKQAFGIMNELRLSQQLCDVTLQVKYQDAPAAQFMAHKVVLASSSPVFKAMFTNGLREQG
MEVVSIEGIHPKVMERLIEFAYTASISMGEKCVLHVMNGAVMYQIDSVVRACSDFLVQQLDP
SNAIGIANFAEQIGCVELHQRAREYIYMHFGEVAKQEEFFNLSHCQLVTLISRDDLNVRCES
EVFHACINWVKYDCEQRRFYVQALLRAVRCHSLTPNFLQMQLQKCEILQSDSRCKDYLVKI
FEELTLHKPTQVMPCRAPKVGRLIYTAGGYFRQSLSYLEAYNPSDGTWLRLADLQVPRSGL
AGCVVGGLLYAVGGRNNSPDGNTDSSALDCYNPMTNQWSPCAPMSVPRNRIGVGVIDGH
IYAVGGSHGCIHHNSVERYEPERDEWHLVAPMLTRRIGVGVAVLNRLLYAVGGFDGTNRLN
SAECYYPERNEWRMITAMNTIRSGAGVCVLHNCIYAAGGYDGQDQLNSVERYDVETETWT
FVAPMKHRRSALGITVHQGRIYVLGGYDGHTFLDSVECYDPDTDTWSEVTRMTSGRSGVG
VAVTMEPCRKQIDQQNCTC*
DNA sequence encoding CRBN (SEQ ID NO: 8):
ATGGCCGGCGAAGGAGATCAGCAGGACGCTGCGCACAACATGGGCAACCACCTGCCG
CTCCTGCCTGCAGAGAGTGAGGAAGAAGATGAAATGGAAGTTGAAGACCAGGATAGTA
AAGAAGCCAAAAAACCAAACATCATAAATTTTGACACCAGTCTGCCGACATCACATACAT
ACCTAGGTGCTGATATGGAAGAATTTCATGGCAGGACTTTGCACGATGACGACAGCTGT
CAGGTGATTCCAGTTCTTCCACAAGTGATGATGATCCTGATTCCCGGACAGACATTACC
TCTTCAGCTTTTTCACCCTCAAGAAGTCAGTATGGTGCGGAATTTAATTCAGAAAGATAG
AACCTTTGCTGTTCTTGCATACAGCAATGTACAGGAAAGGGAAGCACAGTTTGGAACAA
CAGCAGAGATATATGCCTATCGAGAAGAACAGGATTTTGGAATTGAGATAGTGAAAGTG
AAAGCAATTGGAAGACAAAGGTTCAAAGTCCTTGAGCTAAGAACACAGTCAGATGGAAT
CCAGCAAGCTAAAGTGCAAATTCTTCCCGAATGTGTGTTGCCTTCAACCATGTCTGCAG
TTCAATTAGAATCCCTCAATAAGTGCCAGATATTTCCTTCAAAACCTGTCTCAAGAGAAG
ACCAATGTTCATATAAATGGTGGCAGAAATACCAGAAGAGAAAGTTTCATTGTGCAAATC
TAACTTCATGGCCTCGCTGGCTGTATTCCTTATATGATGCTGAGACCTTAATGGACAGA
ATCAAGAAACAGCTACGTGAATGGGATGAAAATCTAAAAGATGATTCTCTTCCTTCAAAT
CCAATAGATTTTTCTTACAGAGTAGCTGCTTGTCTTCCTATTGATGATGTATTGAGAATT
CAGCTCCTTAAAATTGGCAGTGCTATCCAGCGACTTCGCTGTGAATTAGACATTATGAA
TAAATGTACTTCCCTTTGCTGTAAACAATGTCAAGAAACAGAAATAACAACCAAAAATGA
AATATTCAGTTTATCCTTATGTGGGCCGATGGCAGCTTATGTGAATCCTCATGGATATGT
GCATGAGACACTTACTGTGTATAAGGCTTGCAACTTGAATCTGATAGGCCGGCCTTCTA
CAGAACACAGCTGGTTTCCTGGGTATGCCTGGACTGTTGCCCAGTGTAAGATCTGTGC
AAGCCATATTGGATGGAAGTTTACGGCCACCAAAAAAGACATGTCACCTCAAAAATTTT
GGGGCTTAACGCGATCTGCTCTGTTGCCCACGATCCCAGACACTGAAGATGAAATAAG
TCCAGACAAAGTAATACTTTGCTTGTAA
CRBN amino acid sequence (SEQ ID NO: 17):
MAGEGDQQDAAHNMGNHLPLLPAESEEEDEMEVEDQDSKEAKKPNIINFDTSLPTSHTYL
GADMEEFHGRTLHDDDSCQVIPVLPQVMMILIPGQTLPLQLFHPQEVSMVRNLIQKDRTFA
VLAYSNVQEREAQFGTTAEIYAYREEQDFGIEIVKVKAIGRQRFKVLELRTQSDGIQQAKVQI
LPECVLPSTMSAVQLESLNKCQIFPSKPVSREDQCSYKWWQKYQKRKFHCANLTSWPRW
LYSLYDAETLMDRIKKQLREWDENLKDDSLPSNPIDFSYRVAACLPIDDVLRIQLLKIGSAIQR
LRCELDIMNKCTSLCCKQCQETEITTKNEIFSLSLCGPMAAYVNPHGYVHETLTVYKACNLN
LIGRPSTEHSWFPGYAWTVAQCKICASHIGWKFTATKKDMSPQKFWGLTRSALLPTIPDTE
DEISPDKVILCL*
DNA sequence encoding KLHDC2 (SEQ ID NO: 9):
ATGGCTGATGGCAACGAGGATCTGCGGGCTGACGACTTGCCTGGGCCAGCCTTCGAG
AGCTATGAGTCCATGGAGCTTGCCTGCCCCGCTGAGCGCAGCGGCCACGTAGCCGTC
AGCGACGGGCGCCACATGTTCGTCTGGGGCGGCTACAAGAGTAATCAAGTCAGAGGA
TTATATGACTTTTATCTGCCTAGAGAAGAACTATGGATCTACAACATGGAGACTGGAAGA
TGGAAAAAAATCAACACTGAAGGTGATGTTCCTCCTTCTATGTCAGGAAGCTGTGCTGT
GTGTGTAGACAGGGTGCTGTACTTGTTTGGAGGACACCATTCAAGAGGCAATACCAATA
AGTTCTACATGCTGGATTCAAGGTCTACAGACAGAGTGTTACAGTGGGAAAGAATTGAT
TGCCAAGGAATTCCTCCATCATCAAAGGACAAACTTGGTGTCTGGGTATATAAAAACAA
GTTAATATTTTTTGGAGGGTATGGATATTTGCCTGAAGATAAAGTATTGGGAACTTTTGA
ATTCGATGAAACATCTTTTTGGAATTCAAGTCATCCAAGAGGATGGAATGATCATGTACA
TATTTTAGATACTGAAACATTTACCTGGAGCCAGCCTATAACTACTGGTAAAGCACCTTC
ACCTCGTGCTGCCCATGCCTGTGCAACTGTCGGAAATAGAGGCTTCGTGTTTGGAGGC
AGATATCGAGATGCTAGAATGAATGATCTTCACTATCTTAATCTGGATACATGGGAGTG
GAATGAATTAATTCCACAAGGCATATGCCCAGTTGGTCGATCTTGGCACTCACTAACAC
CAGTTTCTTCAGATCATCTTTTTCTCTTTGGAGGATTTACCACTGATAAACAGCCACTAA
GTGATGCCTGGACTTACTGCATCAGTAAAAATGAATGGATACAATTTAATCATCCATATA
CCGAAAAACCAAGGTTATGGCACACAGCTTGTGCCAGCGATGAAGGAGAAGTAATTGT
TTTTGGTGGATGTGCCAACAACTTGCTTGTCCATCACAGAGCTGCACACAGTAATGAAA
TACTAATATTTTCAGTTCAACCAAAATCTCTTGTACGGCTAAGCTTAGAAGCAGTCATTT
GCTTTAAAGAAATGTTAGCCAACTCATGGAACTGCCTTCCAAAACACTTACTTCACAGTG
TTAATCAGAGGTTTGGTAGTAACAACACTTCTGGATCTTAA
KLHDC2 amino acid sequence (SEQ ID NO: 18):
MADGNEDLRADDLPGPAFESYESMELACPAERSGHVAVSDGRHMFVWGGYKSNQVRGL
YDFYLPREELWIYNMETGRWKKINTEGDVPPSMSGSCAVCVDRVLYLFGGHHSRGNTNKF
YMLDSRSTDRVLQWERIDCQGIPPSSKDKLGVWVYKNKLIFFGGYGYLPEDKVLGTFEFDE
TSFWNSSHPRGWNDHVHILDTETFTWSQPITTGKAPSPRAAHACATVGNRGFVFGGRYRD
ARMNDLHYLNLDTWEWNELIPQGICPVGRSWHSLTPVSSDHLFLFGGFTTDKQPLSDAWT
YCISKNEWIQFNHPYTEKPRLWHTACASDEGEVIVFGGCANNLLVHHRAAHSNEILIFSVQP
KSLVRLSLEAVICFKEMLANSWNCLPKHLLHSVNQRFGSNNTSGS*
DNA sequence encoding TRAF3d56 (SEQ ID NO: 10):
ATGGAGTCGAGTAAAAAGATGGACTCTCCTGGCGCGCTGCAGACTAACCCGCCGCTAA
AGCTGCACACTGACCGCAGTGCTGGGACGCCAGTTTTTGTCCCTGAACAAGGAGGTTA
CAAGGAAAAGTTTGTGAAGACCGTGGAGGACAAGTACAAGTGTGAGAAGTGCCACCTG
GTGCTGTGCAGCCCGAAGCAGACCGAGTGTGGGCACCGCTTCTGCGAGAGCTGCATG
GCGGCCCTGCTGAGCTCTTCAAGTCCAAAATGTACAGCGTGTCAAGAGAGCATCGTTA
AAGATAAGGTGTTTAAGGATAATTGCTGCAAGAGAGAAATTCTGGCTCTTCAGATCTATT
GTCGGAATGAAAGCAGAGGTTGTGCAGAGCAGTTAATGCTGGGACATCTGCTGGTGCA
TTTAAAAAATGATTGCCATTTTGAAGAACTTCCATGTGTGCGTCCTGACTGCAAAGAAAA
GGTCTTGAGGAAAGACCTGCGAGACCACGTGGAGAAGGCGTGTAAATACCGGGAAGC
CACATGCAGCCACTGCAAGAGTCAGGTTCCGATGATCGCGCTGCAGAAACACGAAGAC
ACCGACTGTCCCTGCGTGGTGGTGTCCTGCCCTCACAAGTGCAGCGTCCAGACTCTCC
TGAGGAGCGAGGTTTCCTTGTTGCAGAATGAAAGTGTAGAAAAAAACAAGAGCATACAA
AGTTTGCACAATCAGATATGTAGCTTTGAAATTGAAATTGAGAGACAAAAGGAAATGCTT
CGAAATAATGAATCCAAAATCCTTCATTTACAGCGAGTGATAGACAGCCAAGCAGAGAA
ACTGAAGGAGCTTGACAAGGAGATCCGGCCCTTCCGGCAGAACTGGGAGGAAGCAGA
CAGCATGAAGAGCAGCGTGGAGTCCCTCCAGAACCGCGTGACCGAGCTGGAGAGCGT
GGACAAGAGCGCGGGGCAAGTGGCTCGGAACACAGGCCTGCTGGAGTCCCAGCTGA
GCCGGCATGACCAGATGCTGAGTGTGCACGACATCCGCCTAGCCGACATGGACCTGC
GCTTCCAGGTCCTGGAGACCGCCAGCTACAATGGAGTGCTCATCTGGAAGATTCGCGA
CTACAAGCGGCGGAAGCAGGAGGCCGTCATGGGGAAGACCCTGTCCCTTTACAGCCA
GCCTTTCTACACTGGTTACTTTGGCTATAAGATGTGTGCCAGGGTCTACCTGAACGGGG
ACGGGATGGGGAAGGGGACGCACTTGTCGCTGTTTTTTGTCATCATGCGTGGAGAATA
TGATGCCCTGCTTCCTTGGCCGTTTAAGCAGAAAGTGACACTCATGCTGATGGATCAGG
GGTCCTCTCGACGTCATTTGGGAGATGCATTCAAGCCCGACCCCAACAGCAGCAGCTT
CAAGAAGCCCACTGGAGAGATGAATATCGCCTCTGGCTGCCCAGTCTTTGTGGCCCAA
ACTGTTCTAGAAAATGGGACATATATTAAAGATGATACAATTTTTATTAAAGTCATAGTGG
ATACTTCGGATCTGCCCGATCCCTGA
TRAF3d56 amino acid sequence (SEQ ID NO: 19):
MESSKKMDSPGALQTNPPLKLHTDRSAGTPVFVPEQGGYKEKFVKTVEDKYKCEKCHLVL
CSPKQTECGHRFCESCMAALLSSSSPKCTACQESIVKDKVFKDNCCKREILALQIYCRNES
RGCAEQLMLGHLLVHLKNDCHFEELPCVRPDCKEKVLRKDLRDHVEKACKYREATCSHCK
SQVPMIALQKHEDTDCPCVVVSCPHKCSVQTLLRSEVSLLQNESVEKNKSIQSLHNQICSFE
IEIERQKEMLRNNESKILHLQRVIDSQAEKLKELDKEIRPFRQNWEEADSMKSSVESLQNRV
TELESVDKSAGQVARNTGLLESQLSRHDQMLSVHDIRLADMDLRFQVLETASYNGVLIWKI
RDYKRRKQEAVMGKTLSLYSQPFYTGYFGYKMCARVYLNGDGMGKGTHLSLFFVIMRGEY
DALLPWPFKQKVTLMLMDQGSSRRHLGDAFKPDPNSSSFKKPTGEMNIASGCPVFVAQTV
LENGTYIKDDTIFIKVIVDTSDLPDP*
α-synuclein:
MDVFMKGLSKAKEGVVAAAEKTKQGVAEAAGKTKEGVLYVGSKTKEGVVHGVATVAEKTK
EQVTNVGGAVVTGVTAVAQKTVEGAGSIAAATGFVKKDQLGKNEEGAPQEGILEDMPVDP
DNEAYEMPSEEGYQDYEPEA (SEQ ID NO: 11)
NbSYN87 (5-G linker) KEAP1 DNA sequence (SEQ ID NO: 20)
caggtgcagctgcaggaaagcggcggcggcagcgtgcagaccggcggcagcctgcgcctgagctgcgtggcgagcggcta
tagcggctatatggcgtggtttcgccaggcgccgggcaaagaacgcgaaggcattgcggcgatttatcgcggcgataaaattac
ctattatgcgcatagcgtgcagggccgctttaccattagccaggcgaacgcgaaaaacaccgtgtatctgctgatgaacagcctg
aaaccggaagataccgcgatttattattgcgcggcgcgccgcgtggtggcggatagcccgctgctgagcaaaacctatgcgtat
tggggccagggcacccaggtgaccgtgagcagcggtggaggcggaggtATGCAGCCAGATCCCAGGCCTAG
CGGGGCTGGGGCCTGCTGCCGATTCCTGCCCCTGCAGTCACAGTGCCCTGAGGGGGC
AGGGGACGCGGTGATGTACGCCTCCACTGAGTGCAAGGCGGAGGTGACGCCCTCCCA
GCATGGCAACCGCACCTTCAGCTACACCCTGGAGGATCATACCAAGCAGGCCTTTGGC
ATCATGAACGAGCTGCGGCTCAGCCAGCAGCTGTGTGACGTCACACTGCAGGTCAAGT
ACCAGGATGCACCGGCCGCCCAGTTCATGGCCCACAAGGTGGTGCTGGCCTCATCCA
GCCCTGTCTTCAAGGCCATGTTCACCAACGGGCTGCGGGAGCAGGGCATGGAGGTGG
TGTCCATTGAGGGTATCCACCCCAAGGTCATGGAGCGCCTCATTGAATTCGCCTACAC
GGCCTCCATCTCCATGGGCGAGAAGTGTGTCCTCCACGTCATGAACGGTGCTGTCATG
TACCAGATCGACAGCGTTGTCCGTGCCTGCAGTGACTTCCTGGTGCAGCAGCTGGACC
CCAGCAATGCCATCGGCATCGCCAACTTCGCTGAGCAGATTGGCTGTGTGGAGTTGCA
CCAGCGTGCCCGGGAGTACATCTACATGCATTTTGGGGAGGTGGCCAAGCAAGAGGA
GTTCTTCAACCTGTCCCACTGCCAACTGGTGACCCTCATCAGCCGGGACGACCTGAAC
GTGCGCTGCGAGTCCGAGGTCTTCCACGCCTGCATCAACTGGGTCAAGTACGACTGCG
AACAGCGACGGTTCTACGTCCAGGCGCTGCTGCGGGCCGTGCGCTGCCACTCGTTGA
CGCCGAACTTCCTGCAGATGCAGCTGCAGAAGTGCGAGATCCTGCAGTCCGACTCCCG
CTGCAAGGACTACCTGGTCAAGATCTTCGAGGAGCTCACCCTGCACAAGCCCACGCAG
GTGATGCCCTGCCGGGCGCCCAAGGTGGGCCGCCTGATCTACACCGCGGGCGGCTA
CTTCCGACAGTCGCTCAGCTACCTGGAGGCTTACAACCCCAGTGACGGCACCTGGCTC
CGGTTGGCGGACCTGCAGGTGCCGCGGAGCGGCCTGGCCGGCTGCGTGGTGGGCGG
GCTGTTGTACGCCGTGGGCGGCAGGAACAACTCGCCCGACGGCAACACCGACTCCAG
CGCCCTGGACTGTTACAACCCCATGACCAATCAGTGGTCGCCCTGCGCCCCCATGAGC
GTGCCCCGTAACCGCATCGGGGGGGGGTCATCGATGGCCACATCTATGCCGTCGGC
GGCTCCCACGGCTGCATCCACCACAACAGTGTGGAGAGGTATGAGCCAGAGCGGGAT
GAGTGGCACTTGGTGGCCCCAATGCTGACACGAAGGATCGGGGTGGGCGTGGCTGTC
CTCAATCGTCTCCTTTATGCCGTGGGGGGCTTTGACGGGACAAACCGCCTTAATTCAG
CTGAGTGTTACTACCCAGAGAGGAACGAGTGGCGAATGATCACAGCAATGAACACCAT
CCGAAGCGGGGCAGGCGTCTGCGTCCTGCACAACTGTATCTATGCTGCTGGGGGCTA
TGATGGTCAGGACCAGCTGAACAGCGTGGAGCGCTACGATGTGGAAACAGAGACGTG
GACTTTCGTAGCCCCCATGAAGCACCGGCGAAGTGCCCTGGGGATCACTGTCCACCAG
GGGAGAATCTACGTCCTTGGAGGCTATGATGGTCACACGTTCCTGGACAGTGTGGAGT
GTTACGACCCAGATACAGACACCTGGAGCGAGGTGACCCGAATGACATCGGGCCGGA
GTGGGGTGGGCGTGGCTGTCACCATGGAGCCCTGCCGGAAGCAGATTGACCAGCAGA
ACTGTACCTGTTGA
NbSYN87 (5-G linker) KEAP1 (SEQ ID NO: 21
QVQLQESGGGSVQTGGSLRLSCVASGYSGYMAWFRQAPGKEREGIAAIYRGDKITYYAHS
VQGRFTISQANAKNTVYLLMNSLKPEDTAIYYCAARRVVADSPLLSKTYAYWGQGTQVTVS
SGGGGGMQPDPRPSGAGACCRFLPLQSQCPEGAGDAVMYASTECKAEVTPSQHGNRTF
SYTLEDHTKQAFGIMNELRLSQQLCDVTLQVKYQDAPAAQFMAHKVVLASSSPVFKAMFTN
GLREQGMEVVSIEGIHPKVMERLIEFAYTASISMGEKCVLHVMNGAVMYQIDSVVRACSDFL
VQQLDPSNAIGIANFAEQIGCVELHQRAREYIYMHFGEVAKQEEFFNLSHCQLVTLISRDDL
NVRCESEVFHACINWVKYDCEQRRFYVQALLRAVRCHSLTPNFLQMQLQKCEILQSDSRC
KDYLVKIFEELTLHKPTQVMPCRAPKVGRLIYTAGGYFRQSLSYLEAYNPSDGTWLRLADLQ
VPRSGLAGCVVGGLLYAVGGRNNSPDGNTDSSALDCYNPMTNQWSPCAPMSVPRNRIGV
GVIDGHIYAVGGSHGCIHHNSVERYEPERDEWHLVAPMLTRRIGVGVAVLNRLLYAVGGFD
GTNRLNSAECYYPERNEWRMITAMNTIRSGAGVCVLHNCIYAAGGYDGQDQLNSVERYDV
ETETWTFVAPMKHRRSALGITVHQGRIYVLGGYDGHTFLDSVECYDPDTDTWSEVTRMTS
GRSGVGVAVTMEPCRKQIDQQNCTC*
KEAP1 (5-G linker) NbSYN87 DNA sequence (SEQ ID NO: 22)
ATGCAGCCAGATCCCAGGCCTAGCGGGGCTGGGGCCTGCTGCCGATTCCTGCCCCTG
CAGTCACAGTGCCCTGAGGGGGCAGGGGACGCGGTGATGTACGCCTCCACTGAGTGC
AAGGCGGAGGTGACGCCCTCCCAGCATGGCAACCGCACCTTCAGCTACACCCTGGAG
GATCATACCAAGCAGGCCTTTGGCATCATGAACGAGCTGCGGCTCAGCCAGCAGCTGT
GTGACGTCACACTGCAGGTCAAGTACCAGGATGCACCGGCCGCCCAGTTCATGGCCC
ACAAGGTGGTGCTGGCCTCATCCAGCCCTGTCTTCAAGGCCATGTTCACCAACGGGCT
GCGGGAGCAGGGCATGGAGGTGGTGTCCATTGAGGGTATCCACCCCAAGGTCATGGA
GCGCCTCATTGAATTCGCCTACACGGCCTCCATCTCCATGGGCGAGAAGTGTGTCCTC
CACGTCATGAACGGTGCTGTCATGTACCAGATCGACAGCGTTGTCCGTGCCTGCAGTG
ACTTCCTGGTGCAGCAGCTGGACCCCAGCAATGCCATCGGCATCGCCAACTTCGCTGA
GCAGATTGGCTGTGTGGAGTTGCACCAGCGTGCCCGGGAGTACATCTACATGCATTTT
GGGGAGGTGGCCAAGCAAGAGGAGTTCTTCAACCTGTCCCACTGCCAACTGGTGACC
CTCATCAGCCGGGACGACCTGAACGTGCGCTGCGAGTCCGAGGTCTTCCACGCCTGC
ATCAACTGGGTCAAGTACGACTGCGAACAGCGACGGTTCTACGTCCAGGCGCTGCTGC
GGGCCGTGCGCTGCCACTCGTTGACGCCGAACTTCCTGCAGATGCAGCTGCAGAAGT
GCGAGATCCTGCAGTCCGACTCCCGCTGCAAGGACTACCTGGTCAAGATCTTCGAGGA
GCTCACCCTGCACAAGCCCACGCAGGTGATGCCCTGCCGGGCGCCCAAGGTGGGCC
GCCTGATCTACACCGCGGGCGGCTACTTCCGACAGTCGCTCAGCTACCTGGAGGCTTA
CAACCCCAGTGACGGCACCTGGCTCCGGTTGGCGGACCTGCAGGTGCCGCGGAGCG
GCCTGGCCGGCTGCGTGGTGGGCGGGCTGTTGTACGCCGTGGGCGGCAGGAACAAC
TCGCCCGACGGCAACACCGACTCCAGCGCCCTGGACTGTTACAACCCCATGACCAATC
AGTGGTCGCCCTGCGCCCCCATGAGCGTGCCCCGTAACCGCATCGGGGTGGGGGTCA
TCGATGGCCACATCTATGCCGTCGGCGGCTCCCACGGCTGCATCCACCACAACAGTGT
GGAGAGGTATGAGCCAGAGCGGGATGAGTGGCACTTGGTGGCCCCAATGCTGACACG
AAGGATCGGGGTGGGCGTGGCTGTCCTCAATCGTCTCCTTTATGCCGTGGGGGGCTTT
GACGGGACAAACCGCCTTAATTCAGCTGAGTGTTACTACCCAGAGAGGAACGAGTGGC
GAATGATCACAGCAATGAACACCATCCGAAGCGGGGCAGGCGTCTGCGTCCTGCACAA
CTGTATCTATGCTGCTGGGGGCTATGATGGTCAGGACCAGCTGAACAGCGTGGAGCGC
TACGATGTGGAAACAGAGACGTGGACTTTCGTAGCCCCCATGAAGCACCGGCGAAGTG
CCCTGGGGATCACTGTCCACCAGGGGAGAATCTACGTCCTTGGAGGCTATGATGGTCA
CACGTTCCTGGACAGTGTGGAGTGTTACGACCCAGATACAGACACCTGGAGCGAGGTG
ACCCGAATGACATCGGGCCGGAGTGGGGTGGGCGTGGCTGTCACCATGGAGCCCTGC
CGGAAGCAGATTGACCAGCAGAACTGTACCTGTggtggaggcggaggtcaggtgcagctgcaggaaa
gcggcggcggcagcgtgcagaccggcggcagcctgcgcctgagctgcgtggcgagcggctatagcggctatatggcgtggttt
cgccaggcgccgggcaaagaacgcgaaggcattgcggcgatttatcgcggcgataaaattacctattatgcgcatagcgtgca
gggccgctttaccattagccaggcgaacgcgaaaaacaccgtgtatctgctgatgaacagcctgaaaccggaagataccgcg
atttattattgcgcggcgcgccgcgtggtggcggatagcccgctgctgagcaaaacctatgcgtattggggccagggcacccag
gtgaccgtgagcagc
KEAP1 (5-G linker) NbSYN87 amino acid sequence (SEQ ID NO: 23)
MQPDPRPSGAGACCRFLPLQSQCPEGAGDAVMYASTECKAEVTPSQHGNRTFSYTLEDH
TKQAFGIMNELRLSQQLCDVTLQVKYQDAPAAQFMAHKVVLASSSPVFKAMFTNGLREQG
MEVVSIEGIHPKVMERLIEFAYTASISMGEKCVLHVMNGAVMYQIDSVVRACSDFLVQQLDP
SNAIGIANFAEQIGCVELHQRAREYIYMHFGEVAKQEEFFNLSHCQLVTLISRDDLNVRCES
EVFHACINWVKYDCEQRRFYVQALLRAVRCHSLTPNFLQMQLQKCEILQSDSRCKDYLVKI
FEELTLHKPTQVMPCRAPKVGRLIYTAGGYFRQSLSYLEAYNPSDGTWLRLADLQVPRSGL
AGCVVGGLLYAVGGRNNSPDGNTDSSALDCYNPMTNQWSPCAPMSVPRNRIGVGVIDGH
IYAVGGSHGCIHHNSVERYEPERDEWHLVAPMLTRRIGVGVAVLNRLLYAVGGFDGTNRLN
SAECYYPERNEWRMITAMNTIRSGAGVCVLHNCIYAAGGYDGQDQLNSVERYDVETETWT
FVAPMKHRRSALGITVHQGRIYVLGGYDGHTFLDSVECYDPDTDTWSEVTRMTSGRSGVG
VAVTMEPCRKQIDQQNCTCGGGGGQVQLQESGGGSVQTGGSLRLSCVASGYSGYMAWF
RQAPGKEREGIAAIYRGDKITYYAHSVQGRFTISQANAKNTVYLLMNSLKPEDTAIYYCAAR
RVVADSPLLSKTYAYWGQGTQVTVSS
KLHDC2 (5-G linker) NbSYN87 DNA sequence (SEQ ID NO: 24)
ATGGCTGATGGCAACGAGGATCTGCGGGCTGACGACTTGCCTGGGCCAGCCTTCGAG
AGCTATGAGTCCATGGAGCTTGCCTGCCCCGCTGAGCGCAGCGGCCACGTAGCCGTC
AGCGACGGGCGCCACATGTTCGTCTGGGGCGGCTACAAGAGTAATCAAGTCAGAGGA
TTATATGACTTTTATCTGCCTAGAGAAGAACTATGGATCTACAACATGGAGACTGGAAGA
TGGAAAAAAATCAACACTGAAGGTGATGTTCCTCCTTCTATGTCAGGAAGCTGTGCTGT
GTGTGTAGACAGGGTGCTGTACTTGTTTGGAGGACACCATTCAAGAGGCAATACCAATA
AGTTCTACATGCTGGATTCAAGGTCTACAGACAGAGTGTTACAGTGGGAAAGAATTGAT
TGCCAAGGAATTCCTCCATCATCAAAGGACAAACTTGGTGTCTGGGTATATAAAAACAA
GTTAATATTTTTTGGAGGGTATGGATATTTGCCTGAAGATAAAGTATTGGGAACTTTTGA
ATTCGATGAAACATCTTTTTGGAATTCAAGTCATCCAAGAGGATGGAATGATCATGTACA
TATTTTAGATACTGAAACATTTACCTGGAGCCAGCCTATAACTACTGGTAAAGCACCTTC
ACCTCGTGCTGCCCATGCCTGTGCAACTGTCGGAAATAGAGGCTTCGTGTTTGGAGGC
AGATATCGAGATGCTAGAATGAATGATCTTCACTATCTTAATCTGGATACATGGGAGTG
GAATGAATTAATTCCACAAGGCATATGCCCAGTTGGTCGATCTTGGCACTCACTAACAC
CAGTTTCTTCAGATCATCTTTTTCTCTTTGGAGGATTTACCACTGATAAACAGCCACTAA
GTGATGCCTGGACTTACTGCATCAGTAAAAATGAATGGATACAATTTAATCATCCATATA
CCGAAAAACCAAGGTTATGGCACACAGCTTGTGCCAGCGATGAAGGAGAAGTAATTGT
TTTTGGTGGATGTGCCAACAACTTGCTTGTCCATCACAGAGCTGCACACAGTAATGAAA
TACTAATATTTTCAGTTCAACCAAAATCTCTTGTACGGCTAAGCTTAGAAGCAGTCATTT
GCTTTAAAGAAATGTTAGCCAACTCATGGAACTGCCTTCCAAAACACTTACTTCACAGTG
TTAATCAGAGGTTTGGTAGTAACAACACTTCTGGATCTggtggaggcggaggtcaggtgcagctgca
ggaaagcggcggcggcagcgtgcagaccggcggcagcctgcgcctgagctgcgtggcgagcggctatagcggctatatggc
gtggtttcgccaggcgccgggcaaagaacgcgaaggcattgcggcgatttatcgcggcgataaaattacctattatgcgcatag
cgtgcagggccgctttaccattagccaggcgaacgcgaaaaacaccgtgtatctgctgatgaacagcctgaaaccggaagat
accgcgatttattattgcgcggcgcgccgcgtggtggcggatagcccgctgctgagcaaaacctatgcgtattggggccagggc
acccaggtgaccgtgagcagc
KLHDC2 (5-G linker) NbSYN87 amino acid sequence (SEQ ID NO: 25)
MADGNEDLRADDLPGPAFESYESMELACPAERSGHVAVSDGRHMFVWGGYKSNQVRGL
YDFYLPREELWIYNMETGRWKKINTEGDVPPSMSGSCAVCVDRVLYLFGGHHSRGNTNKF
YMLDSRSTDRVLQWERIDCQGIPPSSKDKLGVWVYKNKLIFFGGYGYLPEDKVLGTFEFDE
TSFWNSSHPRGWNDHVHILDTETFTWSQPITTGKAPSPRAAHACATVGNRGFVFGGRYRD
ARMNDLHYLNLDTWEWNELIPQGICPVGRSWHSLTPVSSDHLFLFGGFTTDKQPLSDAWT
YCISKNEWIQFNHPYTEKPRLWHTACASDEGEVIVFGGCANNLLVHHRAAHSNEILIFSVQP
KSLVRLSLEAVICFKEMLANSWNCLPKHLLHSVNQRFGSNNTSGSGGGGGQVQLQESGG
GSVQTGGSLRLSCVASGYSGYMAWFRQAPGKEREGIAAIYRGDKITYYAHSVQGRFTISQA
NAKNTVYLLMNSLKPEDTAIYYCAARRVVADSPLLSKTYAYWGQGTQVTVSS
KLHL6 (5-G linker) NbSYN87 DNA sequence (SEQ ID NO: 26)
ATGTTGATGGCAGGACAAAGGGGCGCCTGGACCATGGGTGATGTGGTCGAGAAGAGT
TTGGAAGGGCCCCTGGCACCTTCTACAGATGAGCCCTCCCAGAAAACAGGAGACTTGG
TCGAGATCTTAAATGGGGAAAAGGTCAAATTTGACGACGCGGGACTCTCCTTAATTCTT
CAGAATGGCCTGGAAACCCTGCGAATGGAAAACGCTCTGACAGATGTCATCTTGTGTG
TGGACATTCAGGAATTCTCCTGCCACCGCGTGGTGCTTGCCGCAGCCAGCAACTATTT
CAGGGCCATGTTCTGCAACGATTTAAAGGAAAAGTATGAGAAAAGGATCATTATTAAAG
GGGTTGATGCTGAGACCATGCACACTCTGTTGGACTACACGTACACCAGCAAGGCGCT
GATCACCAAGCAGAATGTCCAGCGGGTCCTGGAGGCTGCTAACCTCTTCCAGTTCCTG
CGGATGGTGGATGCCTGTGCCAGCTTCCTCACTGAAGCCTTGAACCCAGAAAACTGCG
TTGGAATACTGAGGCTGGCTGACACACACTCGCTGGACAGTCTAAAGAAGCAGGTTCA
GAGTTACATCATTCAAAACTTTGTGCAGATTCTGAACTCTGAGGAGTTTCTTGACCTGCC
CGTGGACACTCTGCACCACATCTTGAAGAGTGATGACCTTTACGTGACCGAGGAGGCT
CAGGTGTTTGAGACCGTGATGAGCTGGGTCCGGCACAAGCCATCAGAACGACTCTGCT
TACTCCCCTATGTCCTCGAGAACGTGCGCTTACCGCTTCTGGACCCGTGGTACTTTGTG
GAGACGGTGGAAGCAGATCCTCTCATCAGGCAGTGCCCAGAGGTCTTCCCGCTGCTCC
AGGAAGCCAGGATGTACCACCTTTCTGGCAATGAGATCATTTCGGAACGCACCAAGCC
CAGGATGCATGAGTTCCAGTCTGAGGTGTTCATGATCATTGGCGGCTGCACGAAGGAT
GAACGGTTTGTGGCAGAGGTGACCTGCCTGGACCCCCTGAGGCGCAGCCGCCTGGAG
GTGGCCAAGCTCCCGCTAACAGAGCATGAGCTGGAGAGTGAGAATAAGAAGTGGGTG
GAGTTTGCATGCGTGACATTGAAAAATGAGGTCTACATCTCAGGTGGCAAAGAAACACA
GCATGATGTTTGGAAATATAATTCTTCGATCAACAAGTGGATTCAGATTGAGTATTTAAA
CATAGGCCGCTGGAGGCATAAGATGGTGGTGTTGGGTGGCAAGGTCTATGTGATCGGA
GGCTTTGACGGCTTACAGAGAATCAACAATGTGGAGACCTACGACCCCTTTCACAACTG
CTGGTCAGAGGCCGCACCCCTCCTTGTCCATGTCAGTTCCTTTGCAGCCACCAGCCAT
AAGAAGAAGCTGTATGTGATCGGGGGAGGGCCCAATGGGAAACTGGCCACAGACAAG
ACTCAGTGTTATGACCCTTCCACCAACAAGTGGAGTTTGAAGGCGGCCATGCCCGTGG
AGGCTAAATGCATCAATGCAGTGAGTTTCCGGGACCGCATCTATGTCGTTGGTGGGGC
CATGAGAGCGCTGTACGCCTACAGCCCGCTGGAAGACAGCTGGTGCCTGGTGACCCA
GCTCAGCCACGAGCGGGCCAGCTGCGGTATCGCGCCCTGCAACAACCGGCTCTACAT
CACCGGCGGGCGGGACGAGAAGAACGAGGTTATCGCCACGGTGCTGTGCTGGGACC
CCGAGGCCCAGAAACTGACAGAGGAGTGCGTCCTGCCCCGGGGCGTGTCGCACCACG
GCAGCGTCACCATCAGGAAGTCGTACACCCACATCCGCAGGATCGTGCCCGGAGCAG
TGTCTGTCGGTGGAGGCGGAGGTcaggtgcagctgcaggaaagcggcggcggcagcgtgcagaccggcg
gcagcctgcgcctgagctgcgtggcgagcggctatagcggctatatggcgtggtttcgccaggcgccgggcaaagaacgcga
aggcattgcggcgatttatcgcggcgataaaattacctattatgcgcatagcgtgcagggccgctttaccattagccaggcgaac
gcgaaaaacaccgtgtatctgctgatgaacagcctgaaaccggaagataccgcgatttattattgcgcggcgcgccgcgtggtg
gcggatagcccgctgctgagcaaaacctatgcgtattggggccagggcacccaggtgaccgtgagcagc
KLHL6 (5-G linker) NbSYN87 amino acid sequence (SEQ ID NO: 27)
MLMAGQRGAWTMGDVVEKSLEGPLAPSTDEPSQKTGDLVEILNGEKVKFDDAGLSLILQN
GLETLRMENALTDVILCVDIQEFSCHRVVLAAASNYFRAMFCNDLKEKYEKRIIIKGVDAETM
HTLLDYTYTSKALITKQNVQRVLEAANLFQFLRMVDACASFLTEALNPENCVGILRLADTHSL
DSLKKQVQSYIIQNFVQILNSEEFLDLPVDTLHHILKSDDLYVTEEAQVFETVMSWVRHKPSE
RLCLLPYVLENVRLPLLDPWYFVETVEADPLIRQCPEVFPLLQEARMYHLSGNEIISERTKPR
MHEFQSEVFMIIGGCTKDERFVAEVTCLDPLRRSRLEVAKLPLTEHELESENKKWVEFACV
TLKNEVYISGGKETQHDVWKYNSSINKWIQIEYLNIGRWRHKMVVLGGKVYVIGGFDGLQRI
NNVETYDPFHNCWSEAAPLLVHVSSFAATSHKKKLYVIGGGPNGKLATDKTQCYDPSTNK
WSLKAAMPVEAKCINAVSFRDRIYVVGGAMRALYAYSPLEDSWCLVTQLSHERASCGIAPC
NNRLYITGGRDEKNEVIATVLCWDPEAQKLTEECVLPRGVSHHGSVTIRKSYTHIRRIVPGA
VSVGGGGGQVQLQESGGGSVQTGGSLRLSCVASGYSGYMAWFRQAPGKEREGIAAIYR
GDKITYYAHSVQGRFTISQANAKNTVYLLMNSLKPEDTAIYYCAARRVVADSPLLSKTYAYW
GQGTQVTVSS

### REFERENCES

1. NHS. Parkinson's disease—NHS [Internet]. 2019 [cited 2019 Jun. 25]. Available from: https://www.nhs.uk/conditions/parkinsons-disease/
2. Wakabayashi K, Tanji K, Mori F, Takahashi H. The Lewy body in Parkinson's disease: molecules implicated in the formation and degradation of alpha-synuclein aggregates. Neuropathology [Internet]. 2007 October [cited 2019 Jun. 20]; 27(5):494-506. Available from: http://www.ncbi.nlm.nih.gov/pubmed/18018486
3. Parkkinen L, O'Sullivan S S, Collins C, Petrie A, Holton J L, Revesz T, et al. Disentangling the relationship between lewy bodies and nigral neuronal loss in Parkinson's disease. J Parkinsons Dis [Internet]. 2011 [cited 2019 Jun. 20]; 1(3):277-86. Available from: http://www.ncbi.nlm.nih.gov/pubmed/23939308
4. Goldman J G, Postuma R. Premotor and nonmotor features of Parkinson's disease. Curr Opin Neurol [Internet]. 2014 August [cited 2019 Jun. 17]; 27(4):434-41. Available from: http://www.ncbi.nlm.nih.gov/pubmed/24978368
5. Spillantini M G, Schmidt M L, Lee V M-Y, Trojanowski J Q, Jakes R, Goedert M. α-Synuclein in Lewy bodies. Nature [Internet]. 1997 Aug. 28 [cited 2019 Jun. 5]; 388(6645):839-40. Available from: http://www.ncbi.nlm.nih.gov/pubmed/9278044
6. Narhi L, Wood S J, Steavenson S, Jiang Y, Wu G M, Anafi D, et al. Both Familial Parkinson's Disease Mutations Accelerate α-Synuclein Aggregation. J Biol Chem [Internet]. 1999 Apr. 2 [cited 2019 Jun. 20]; 274(14):9843-6. Available from: http://www.ncbi.nlm.nih.gov/pubmed/10092675
7. Polymeropoulos M H, Lavedan C, Leroy E, Ide S E, Dehejia A, Dutra A, et al. Mutation in the alpha-synuclein gene identified in families with Parkinson's disease. Science [Internet]. 1997 Jun. 27 [cited 2019 Jun. 5]; 276(5321):2045-7. Available from: http://www.ncbi.nlm.nih.gov/pubmed/9197268
8. Appel-Cresswell S, Vilarino-Guell C, Encarnacion M, Sherman H, Yu I, Shah B, et al. Alpha-synuclein p.H50Q, a novel pathogenic mutation for Parkinson's disease. Mov Disord [Internet]. 2013 Jun. 1 [cited 2019 Jun. 5]; 28(6):811-3. Available from: http://doi.wiley.com/10.1002/mds.25421
9. Lesage S, Anheim M, Letournel F, Bousset L, Honore A, Rozas N, et al. G51D α-synuclein mutation causes a novel Parkinsonian-pyramidal syndrome. Ann Neurol [Internet]. 2013 April [cited 2019 Jun. 5]; 73(4):459-71. Available from: http://www.ncbi.nlm.nih.gov/pubmed/23526723
10. Zarranz J J, Alegre J, Gómez-Esteban J C, Lezcano E, Ros R, Ampuero I, et al. The new mutation, E46K, of α-synuclein causes parkinson and Lewy body dementia. Ann Neurol [Internet]. 2004 February [cited 2019 Jun. 5]; 55(2):164-73. Available from: http://www.ncbi.nlm.nih.gov/pubmed/14755719
11. Chartier-Harlin M-C, Kachergus J, Roumier C, Mouroux V, Douay X, Lincoln S, et al. α-synuclein locus duplication as a cause of familial Parkinson's disease. Lancet [Internet]. 2004 Sep. 25 [cited 2019 Jun. 5]; 364(9440):1167-9. Available from: https://www.sciencedirect.com/science/article/pii/SO140673604171031 #bib2
12. Singleton A B, Farrer M, Johnson J, Singleton A, Hague S, Kachergus J, et al. -Synuclein Locus Triplication Causes Parkinson's Disease. Science (80- ) [Internet]. 2003 Oct. 31 [cited 2019 Jun. 5]; 302(5646):841-841. Available from: http://www.ncbi.nlm.nih.gov/pubmed/14593171
13. Yang M-L, Hasadsri L, Woods W S, George J M. Dynamic transport and localization of alpha-synuclein in primary hippocampal neurons. Mol Neurodegener [Internet]. 2010 Feb. 9 [cited 2019 Jun. 25]; 5(1):9. Available from: http://www.ncbi.nlm.nih.gov/pubmed/20181133
14. Burre J, Sharma M, Tsetsenis T, Buchman V, Etherton M R, SOdhof T C. α-Synuclein promotes SNARE-complex assembly in vivo and in vitro. Science (80- ). 2010 Sep. 24; 329(5999):1663-7.
15. Bartels T, Ahlstrom L S, Leftin A, Kamp F, Haass C, Brown M F, et al. The N-Terminus of the Intrinsically Disordered Protein α-Synuclein Triggers Membrane Binding and Helix Folding. Biophys J [Internet]. 2010 October [cited 2019 Jun. 25]; 99(7):2116-24. Available from: https://linkinghub.elsevier.com/retrieve/pii/S0006349510007782
16. Tsigelny I F, Sharikov Y, Kouznetsova V L, Greenberg J P, Wrasidlo W, Overk C, et al. Molecular determinants of α-synuclein mutants' oligomerization and membrane interactions. ACS Chem Neurosci [Internet]. 2015 Mar. 18 [cited 2019 Jun. 20]; 6(3):403-16. Available from: http://www.ncbi.nlm.nih.gov/pubmed/25561023
17. Samuel F, Flavin W P, Iqbal S, Pacelli C, Renganathan SDS, Trudeau L E, et al. Effects of serine 129 phosphorylation on α-synuclein aggregation, membrane association, and internalization. J Biol Chem. 2016 Feb. 26; 291(9):4374-85.
18. Fulcher L J, Macartney T, Bozatzi P, Hornberger A, Rojas-Fernandez A, Sapkota G P. An affinity-directed protein missile system for targeted proteolysis. Open Biol [Internet]. 2016 Oct. 26 [cited 2019 Jun. 3]; 6(10):160255. Available from: http://www.ncbi.nlm.nih.gov/pubmed/27784791
19. Fulcher L J, Hutchinson L D, Macartney T J, Turnbull C, Sapkota G P. Targeting endogenous proteins for degradation through the affinity-directed protein missile system. Open Biol [Internet]. 2017 May 10 [cited 2019 Jun. 3]; 7(5):170066. Available from: http://www.ncbi.nlm.nih.gov/pubmed/28490657
20. Bulatov E, Ciulli A. Targeting Cullin-RING E3 ubiquitin ligases for drug discovery: structure, assembly and small-molecule modulation. Biochem J [Internet]. 2015 May 1 [cited 2019 Jun. 21]; 467(3):365-86. Available from: http://www.ncbi.nlm.nih.gov/pubmed/25886174
21. Duda D M, Scott D C, Calabrese M F, Zimmerman E S, Zheng N, Schulman B A. Structural regulation of cullin-RING ubiquitin ligase complexes. Curr Opin Struct Biol [Internet]. 2011 April [cited 2019 Jun. 10]; 21(2):257-64. Available from: http://www.ncbi.nlm.nih.gov/pubmed/21288713
22. De Genst E J, Guilliams T, Wellens J, Day E M, Waudby C A, Meehan S, et al. Structure and properties of a complex of α-synuclein and a single-domain camelid antibody. J Mol Biol [Internet]. 2010 [cited 2021 Apr. 22]; 402(2):326-43. Available from: https://pubmed.ncbi.nlm.nih.gov/20620148/
23. Guilliams T, E1-Turk F, Buell A K, O'Day E M, Aprile F A, Esbjörner E K, et al. Nanobodies Raised against Monomeric α-Synuclein Distinguish between Fibrils at Different Maturation Stages. J Mol Biol [Internet]. 2013 Jul. 24 [cited 2019 Jun. 27]; 425(14):2397-411. Available from: https://www.sciencedirect.com/science/article/pii/S0022283613001897?via %3Dihub
24. Chatterjee D, Bhatt M, Butler D, De Genst E, Dobson C M, Messer A, et al. Proteasome-targeted nanobodies alleviate pathology and functional decline in an α-synuclein-based Parkinson's disease model. npj Park Dis [Internet]. 2018 Dec. 1 [cited 2020 Jun. 23]; 4(1):1-10. Available from: https://www.nature.com/articles/s41531-018-0062-4
25. Lee B R, Kamitani T. Improved Immunodetection of Endogenous α-Synuclein. Feany M B, editor. PLoS One [Internet]. 2011 Aug. 19 [cited 2019 Jun. 20]; 6 (8):e23939. Available from: https://dx.plos.org/10.1371/journal.pone.0023939
26. Chen Y, Dolt K S, Kriek M, Baker T, Downey P, Drummond N J, et al. Engineering synucleinopathy-resistant human dopaminergic neurons by CRISPR-mediated deletion of the SNCA gene. Eur J Neurosci. 2019 Feb. 1; 49(4):510-24.
27. Klivenyi P, Siwek D, Gardian G, Yang L, Starkov A, Cleren C, et al. Mice lacking alpha-synuclein are resistant to mitochondrial toxins. 2005 [cited 2020 May 28]; Available from: www.elsevier.com/locate/ynbdi
28. Fountaine T M, Wade-Martins R. RNA interference-mediated knockdown of α-synuclein protects human dopaminergic neuroblastoma cells from MPP+toxicity and reduces dopamine transport. J Neurosci Res. 2007 Feb. 1; 85(2):351-63.
29. Dauer W, Kholodilov N, Vila M, Trillat A C, Goodchild R, Larsen K E, et al. Resistance of α-synuclein null mice to the parkinsonian neurotoxin MPTP. Proc Natl Acad Sci USA. 2002 Oct. 29; 99(22):14524-9.
30. Farzadfard, A., Pedersen, J. N., Meisl, G. et al. The C-terminal tail of α-synuclein protects against aggregate replication but is critical for oligomerization. Commun Biol 5, 123 (2022). https://doi.org/10.1038/s42003-022-03059-8

1. A proteasomal degradation protein complex comprising an E3 ubiquitin ligase component tethered to an α-synuclein-specific polypeptide binder; wherein the proteasomal degradation protein complex is capable of targeting α-synuclein for proteasomal degradation.

2. The proteasomal degradation protein complex of claim 1, wherein the α-synuclein-specific polypeptide binder is an antibody, an antibody fragment, a monobody and/or a nanobody.

3. The proteasomal degradation protein complex of claim 1, wherein the α-synuclein-specific polypeptide binder binds to a target epitope in the C-terminal region of the α-synuclein protein, that is capable of degrading α-synuclein.

4. The proteasomal degradation protein complex of claim 1, wherein the α-synuclein-specific polypeptide binder is NbSYN87 or a functional variant thereof which comprises a sequence that is at least 70, 80%, 90%, 95% or 99% identical to NbSYN87;
or wherein the α-synuclein-specific polypeptide binder targets the same epitope on α-synuclein as NbSYN87;
or wherein the α-synuclein-specific polypeptide binder with a similar or higher affinity as NbSYN87.

5. The proteasomal degradation protein complex of claim 1, wherein the E3 ubiquitin ligase component is a Cullin ring E3 ligase complex substrate receptor.

6. The proteasomal degradation protein complex of claim 1, wherein the E3 ubiquitin ligase component is capable of recruiting α-synuclein to any of CUL2-CRL, CUL3 or CUL4.

7. The proteasomal degradation protein complex of claim 1, wherein the E3 ubiquitin ligase component is the von-Hippel Lindau tumor suppressor (VHL) or a functional variant thereof, suitably wherein the functional variant comprises a sequence that is at least 60% identical to wild type VHL, preferably at least 70, 80%, 90%, 95% or 99% identical to wild type VHL.

8. The proteasomal degradation protein complex of claim 1, wherein the α-synuclein protein is targeted for degradation by ubiquitin-mediated proteasomal degradation system.

9. The proteasomal degradation protein complex of claim 1 which is an affinity-directed protein missile (AdPROM).

10. One or more nucleic acid constructs encoding the proteasomal degradation protein complex of claim 1.

11. The one or more nucleic acid constructs of claim 10, wherein the E3 ligase component is VHL or a functional variant thereof and the α-synuclein-specific polypeptide binder is NbSYN87 or a functional variant thereof.

12. The one or more nucleic acid constructs of claim 10, comprising the nucleotide sequence of SEQ ID NO: 3 or a sequence that is at least 60, 70%, 80%, 90%, 95% or 99% identical thereto.

13. An expression construct comprising the nucleic acid construct of claim 10 operably linked to one or more expression control sequences.

14. A vector comprising the expression construct of claim 13.

15. The vector of claim 14, which is a gene therapy vector, suitably a viral vector, suitably an AAV vector, an adenoviral vector, a retroviral vector or a lentiviral vector.

16. A pharmaceutical composition comprising the proteasomal degradation protein complex of claim 1, one or more nucleic acid constructs encoding the proteasomal degradation protein complex, an expression construct comprising the one or more nucleic acid constructs operably linked to one or more expression control sequences, or a vector comprising the expression construct and a pharmaceutically acceptable carrier or diluent.

17. The proteasomal degradation protein complex of claim 1, one or more nucleic acid constructs encoding the proteasomal degradation protein complex, an expression construct comprising the one or more nucleic acid constructs operably linked to one or more expression control sequences, a vector comprising the expression construct, or a pharmaceutical composition comprising the proteasomal degradation protein complex, the one or more nucleic acid constructs, the expression construct, the vector and a pharmaceutically acceptable carrier or diluent for use in the treatment of a subject with a neurodegenerative disorder.

18. The use in accordance with claim 17, wherein the neurodegenerative disorder is a synucleinopathy: or wherein the neurodegenerative disorder is Parkinson's disease, dementia and/or multiple system atrophy; or wherein the proteasomal degradation protein complex targets α-synuclein for proteasomal degradation: or wherein the subject has a SNCA duplication, SNCA triplication or an A53T, A30P, H50Q, E46K, G51D and/or A53E point mutation of SEQ ID NO: 11.

19. (canceled)

20. (canceled)

21. (canceled)

22. A method for targeting α-synuclein for degradation using the proteasomal degradation protein complex of claim 1, one or more nucleic acid constructs encoding the proteasomal degradation protein complex, an expression construct comprising the one or more nucleic acid constructs operably linked to one or more expression control sequences, a vector comprising the expression construct, or a pharmaceutical composition comprising the proteasomal degradation protein complex, the one or more nucleic acid constructs, the expression construct, the vector and a pharmaceutically acceptable carrier or diluent.

23. The method of claim 22, wherein the method comprises
administering the proteasomal degradation protein complex of claim 1, one or more nucleic acid constructs encoding the proteasomal degradation protein complex, an expression construct comprising the one or more nucleic acid constructs operably linked to one or more expression control sequences, a vector comprising the expression construct, or a pharmaceutical composition comprising the proteasomal degradation protein complex, the one or more nucleic acid constructs, the expression construct, the vector and a pharmaceutically acceptable carrier or diluent to a cell in vitro or in vivo;
wherein the α-synuclein-specific polypeptide binder component of the proteasomal degradation protein complex binds to α-synuclein;
the E3 ligase component tethered to the α-synuclein-specific polypeptide binder recruits the α-synuclein protein to E3 ligase system in the cell;
the E3 ligase system in the cell ubiquitinylates α-synuclein such that α-synuclein is degraded.

24. The method of claim 22, wherein the α-synuclein is monomeric, oligomeric, protofibrillar, mature fibrils or aggregated; or wherein the α-synuclein is an A53T, A30P, H50Q, E46K, G51D and/or A53E mutant of SEQ ID NO: 11; or wherein the α-synuclein is targeted for proteasomal degradation.

25. (canceled)

26. (canceled)

27. The method of claim 22, wherein the proteasomal degradation is ubiquitin-mediated proteasomal degradation.

28. A method of treatment of a subject in need thereof, the method comprising administering to said subject a therapeutically effective amount of the proteasomal degradation protein complex of claim 1, one or more nucleic acid constructs encoding the proteasomal degradation protein complex, an expression construct comprising the one or more nucleic acid constructs operably linked to one or more expression control sequences, a vector comprising the expression construct, or a pharmaceutical composition comprising the proteasomal degradation protein complex, the one or more nucleic acid constructs, the expression construct, the vector and a pharmaceutically acceptable carrier or diluent.

29. The method of claim 28 wherein the subject has a neurodegenerative disorder, optionally wherein the neurodegenerative disorder is a synucleinopathy, optionally wherein the neurodegenerative disorder is Parkinson's disease, dementia and/or multiple system atrophy; or wherein the proteasomal degradation protein complex targets α-synuclein for proteasomal degradation, suitably wherein the subject has a SNCA duplication, SNCA triplication or an A53T, A30P, H50Q, E46K, G51D and/or A53E point mutation of SEQ ID NO: 11.

30. (canceled)

31. The proteasomal degradation protein complex of claim 1, one or more nucleic acid constructs encoding the proteasomal degradation protein complex, an expression construct comprising the one or more nucleic acid constructs operably linked to one or more expression control sequences, a vector comprising the expression construct, or a pharmaceutical composition comprising the proteasomal degradation protein complex, the one or more nucleic acid constructs, the expression construct, the vector and a pharmaceutically acceptable carrier or diluent for use as a research tool to target.

32. A kit, comprising the proteasomal degradation protein complex of claim 1, one or more nucleic acid constructs encoding the proteasomal degradation protein complex, an expression construct comprising the one or more nucleic acid constructs operably linked to one or more expression control sequences, a vector comprising the expression construct, or a pharmaceutical composition comprising the proteasomal degradation protein complex, the one or more nucleic acid constructs, the expression construct, the vector and a pharmaceutically acceptable carrier or diluent, and instructions for use.
